(19) United States
(12) Patent Application Publication
(10) Pub. No.: US 2022/0118074 A1
(43) Pub. Date: Apr. 21, 2022

(54) ZIKA VIRUS VACCINES USING VIRUS-LIKE PARTICLES

(71) Applicants: Brock Adam Kingstad-Bakke, Madison, WI (US); Jorge E. Osorio, Mount Horeb, WI (US)

(72) Inventors: Brock Adam Kingstad-Bakke, Madison, WI (US); Jorge E. Osorio, Mount Horeb, WI (US)

(21) Appl. No.: 17/477,077

(22) Filed: Sep. 16, 2021

Related U.S. Application Data
(63) Continuation of application No. 15/629,503, filed on Jun. 21, 2017, now abandoned.
(60) Provisional application No. 62/352,904, filed on Jun. 21, 2016.
(60) Provisional application No. 62/384,967, filed on Sep. 8, 2016.

(51) Int. Cl. A61K 39/12; C12N 7/00; A61P 31/14

(52) U.S. Cl. CPC A61K 39/12; A61K 2039/55; A61P 31/14; C12N 7/00

(57) ABSTRACT

A flavivirus virus-like particle and methods of making and using that particle, and antibodies raised to a plurality of those particles, arc provided.

### CROSS-REFERENCE TO RELATED APPLICATIONS

This application is a continuation of U.S. application Ser. No. 15/629,503, filed Jun. 21, 2017, which claims the benefit of the filing date of U.S. application Ser. No. 62/352,904, filed on Jun. 21, 2016, and U.S. application Ser. No. 62/384,967, filed on Sep. 8, 2016, the disclosure of which are incorporated by reference herein.

### BACKGROUND

Zika virus (ZIKV; Flaviviridae, Flavivirus) is an emerging arbovirus, transmitted by Aedes mosquitoes (Ioos et al., 2014). ZIKV has a positive-sense, single-stranded RNA genome, approximately 11 kilobases in length that encodes three structural proteins: the capsid (C), premembrane/membrane (prM), and envelope (E), and seven non-structural proteins (NS1, NS2A, NS2B, NS3, NS4A, 2K, NS4B, and NS5). Based on a genetic study using nucleotide sequences derived from the NS5 gene, there are three ZIKV lineages: East African, West African, and Asian (Mosso, 2015; Faye et al., 2014). ZIKV emerged out of Africa and previously caused outbreaks of febrile disease in the Yap islands of the Federated states of Micronesia (Duffy et al., 2009), French Polynesia (Cao-Lormeau et al., 2014), and Oceania. Currently, several Latin American countries are experiencing the first-ever reported local transmission of ZIKV in the Americas (Hennessey et al., 2016). The current outbreak in the Americas is cause for great concern, because of the fast and uncontrolled autochthonous spread. Clinically, infection with ZIKV resembles dengue fever and several other arboviral diseases (Dyer, 2015), but it has been linked to neurological syndromes and congenital malformation (Pinto Junior et al., 2015). Alarmingly, the rate of microcephaly (small head, reduced brain size, impaired neurocognitive development) in infants born to pregnant women has increased significantly (20-fold in 2015) in areas with high ZIKV incidence in Brazil (Oliveira Melo et al., 2016) (Butler, 2016). In February 2016, the World Health Organization declared the Zika virus an international public health emergency, prompted by its link to microcephaly. As many as four million people could be infected by the end of the year (Gulland, 2016).

To date, there are no vaccines or antiviral therapy for ZIKV, although successful vaccines have been developed for other flavivirus infections (dengue, Japanese encephalitis and yellow fever).

### SUMMARY

Mosquito-borne Zika virus (ZIKV) typically causes a mild and self-limiting illness known as Zika fever, which often is accompanied by maculopapular rash, headache, and myalgia. However, more serious consequences have been reported for ZIKV infection during pregnancy, e.g., microcephaly of the fetus. As described herein, Zika virus-like particles (VLPs) were developed and their immunogenicity and protective efficacy were evaluated in a small animal model for wild-type ZIKV. The prM and E genes of ZIKV strain 33 H/PF/2013 with a nascent signal sequence in the 3′ coding region of the capsid protein were cloned into a pCMV expression vector under the control of a cytomegalovirus (CMV) promoter and CMV polyadenylation signal. Following transfection of HEK293 cells, ZIKV-VLPs expression was confirmed by Western blot and transmission electron microscopy. ZIKV-VLPs (about 0.45 μg) were formulated with 0.2% Imject alum and used to inject groups of six-week-old AG129 mice by the intramuscular (IM) route, followed by a boost administration two weeks later. Control groups received PBS mixed with alum. At five weeks post-initial vaccination all animals were challenged with 200 PFU (>400 LD₅₀s) of ZIKV strain H/PF/2013 by injection into the right hind footpad. All control animals (n=6) died 9 days post challenge, while vaccinated mice survived with no morbidity or weight loss and had significantly lower viremia. This was in contrast to Dengue VLPs produced from prM and E, which did not produce a protective immune response (Pillman, 2015). Significant levels of neutralizing antibodies were observed in all ZIKV-VLP vaccinated mice compared to control groups. The role of neutralizing antibodies in protecting mice was demonstrated by antibody passive transfer studies; naive AG129 mice that received pooled serum from VIP vaccinated animals were fully protected. Thus, the present findings demonstrate the protective efficacy of the ZIKV-VLP vaccine and highlight the role that neutralizing antibodies play in protection against ZIKV infection.

One advantage of VLPs is that VLPs structurally mimic the conformation of native viruses but do not contain any viral genetic material (no viral replication) and are therefore non-infectious. This is in contrast to a live attenuated vaccine (which has genetic material) or in the case of insufficient inactivation of killed vaccines (resulting in viral replication). A VLP vaccine approach eliminates concerns associated with such replication for pregnant women and other populations at high risk for suffering the effects of ZIKV infections.

In one embodiment, a recombinant nucleic acid vector is provided comprising a heterologous promoter operably linked to a sequence encoding flavivirus, e.g., ZIKV, prM/E. In one embodiment, the vector lacks nucleic acid sequences encoding one or more of flavivirus NS1, NS2A, NS2B, NS3, NS4A, NS4B or NS5 and optionally lacks nucleic acid sequences encoding functional flavivirus capsid, e.g., a protein that aggregates so as to form a viral capsid having a diameter of about 50 to 60 nm or about 45 nm to 70 nm. In one embodiment, the heterologous promoter is expressed in mammalian cells. In one embodiment, the heterologous promoter is a heterologous viral promoter. In one embodiment, the heterologous promoter comprises a CMV promoter, a SV40 promoter, an EF-1α promoter or a PGK1 promoter. In one embodiment, the flavivirus is a Zika virus. In one embodiment, the vector sequences are from a Zika virus from the East African or West African lineage. In one embodiment, only a portion of flavivirus capsid sequences is included, e.g., a CT-terminal portion of a flavivirus capsid that is linked to prM/E sequences as in the polyprotein that is expressed by wild-type flavivirus. In one embodiment, the portion of the capsid sequence includes amino acids 98 to 112 of the capsid protein encoded by SEQ ID NO:1 or a protein having at least 80%, 82%o, 85%, 87%, 90%, 92%, 95%, 97%, 99% or more amino acid sequence identity thereto. In one embodiment, the prM/E sequences have at least 80%%, 82%, 85%, 87%, 90%, 92%, 95%, 97%, 98%, 99% or more amino acid sequence identity to the prM/E sequences encoded by any one of SEQ ID Nos. 1-3, 5 or 11-13. In one embodiment, the portion of the capsid sequence lacks a NS2B-3 cleavage site, e.g., KEKKRR (SEQ ID NO:10). In one embodiment, the prM/E sequences are operably linked to a heterologous secretion signal. In one embodiment, the vector further comprises an intron and/or enhancer sequence, e.g., 5′ to a prM/E coding sequence. In one embodiment, the vector further comprises comprises an intron, internal ribosome entry sequence, or an enhancer sequence, or any combinantion thereof.

A recombinant host cell comprising the vector is also provided. In one embodiment, the cell is a mammalian, e,g, Vero cell, HeLa cell or CHO cell, insect or yeast cell. In one embodiment, the cell is a human or simian cell. In one embodiment, the genome of the cell is augmented, e.g., stably augmented, with nucleic acid sequences encoding flavivirus NS2B, e.g., the source of NS2B may be heterologous or homologous to the source for prM/E. In one embodiment, the genome of the cell is augmented, e.g., stably augmented, with nucleic acid sequences encoding flavivirus capsid, e.g., the capsid may be heterologous or homologous to prM/E, which sequences are optionally integrated into the genome of the cell in one embodiment, the genome of the cell is augmented with nucleic acid sequences encoding flavivuirus NS2B, which sequences are optionally integrated into the genome of the cell. In one embodiment, the vector is integrated into the genome of the host cell.

Also provided is a method to prepare flavivirus VLPs. The method includes contacting a culture of isolated host cells that do not express one or more of flavivirus NS1, NS2A, NS2B, NS3, NS4A, NS4B or NSS and optionally do not express functional flavivirus capsid, with the recombinant vector and collecting VLPs from supernatant of the culture. Thus, in one embodiment, the isolated host cells do not have flavivirus sequences prior to contact with the vector. In one embodiment, the collected particles have a diameter of about 10 to 100 nm, e.g., 20 to 60 nm, 40 to 70 nm or 40 to 60 nm. In one embodiment, the host cell expresses flavivirus NS2B. In one embodiment, the host cell expresses flavivirus capsid protein and optionally NS2B.

Further provided is a preparation comprising a flavivirus VD's. The VLP comprises a lipid bilayer comprising flavivirus prM/E but lacks one or more of a flavivirus NS1, NS2A, NS2B, NS3, NS4A, NS4B or NS5 and optionally lacks functional flavivirus capsid. Such a preparation may be used in a vaccine or immunogenic composition. The vaccine or immunogenic composition may have about 10 μg to 1000 μg, e.g., 200 μg to 400 μg or 400 μg to 800 μg, about 0.5 μg to 100 μg, about 1 μg to 50 μg, about 5 μg to 75 μg, about 1 to 500 mg, e.g., about 20 to 50 mg, about 100 to 300 or about 300 to 400 mg, of VLP. The vaccine or immunogenic composition may further comprise one or more adjuvants, In one embodiment, the adjuvant comprises alum, monophosphoryl lipid A (MPLA), squalene, a TLR4 agonist, dimethyldioctadecylammonium, tripalmitoyl-S-glyceryl cysteine, trehalose dibehenate; saponin, MF59, AS03, virosomes AS04, CpG, imidazoquinoline, poly LC, flagellin, or any combination thereof. In one embodiment, an adjuvant is included at about 0.001 mg to about 10 mg, about 0.01 to about 10 mg, about 1 to about 20 mg, or about 10 mg to about 100 mg.

Further provided is a method to prevent, inhibit or treat flavivirus infection in a mammal. The method includes administering an effective amount of the recombinant vector, a host cell having the vector or the vaccine or immunogenic composition having the VLPs. In one embodiment, the mammal is a female mammal. In one embodiment, the vector, host cell, vaccine or immunogenic composition is administered subcutaneously, intradermally, intramuscularly or intravenously to the mammal.

In one embodiment, a method to passively prevent, inhibit or treat flavivirus infection in a mammal is provided. The method includes obtaining serum or plasma having anti-flavivirus antibodies from a mammal exposed to flavivirus and optionally isolating antibodies from the serum or plasma; and administering an effective amount of the serum or plasma, or isolated antibodies, to a different mammal at risk of or having a flavivirus infection. In one embodiment, the mammal is immunocompromised. In one embodiment, the and-flavivirus antibodies are isolated from the serum before administration. In one embodiment, the mammal is a human.

### BRIEF DESCRIPTION OF THE FIGURES

FIGS. 1A-E. in vitro characterization of Zika virus like particles. A) Schematic of pCMV-prM/E expression cassette. B) Western blot analysis of Zika virus like particles. Lanes are, 1) Bio-rad precision plus kaleidoscope protein standards. 2): pCMV-prM/E transfection pre sucrose cushion purification supe. 3) 3.5×10⁴ PFU ZIKV positive control. 4) pCMV-prM/E transfection post sucrose cushion purification pt. 5) pCMV-GFP transfection post sucrose cushion purification pt. C-E) Sucrose cushion purified Zika VLPs observed using transmission electron microscopy. C) VLPs stained with Tungsten. Diameter is indicated. Background protein staining also apparent. D) VLP stained with Tungsten. Membrane proteins visible on the surface of VLP are indicated with arrow. Background protein staining apparent. E) VLP stained with Uranyl acetate. Membrane proteins visible on the surface of VLP are indicated with an arrow.

FIGS. 2A-F. Protection of ZIKVLPS in AG129 mice. A) Neutralizing antibody titers (+/−SD) of vaccinated AG129 mice pre boost and pre challenge. B) Average weight loss (+/−SD) of AG129 after ID challenge with 200 PFU ZIKV over a 14 day period. C) Survival of 11 week old AG129 after ID challenge with 200 PFU ZIKV over a 14 day period. D) Viremia (+/−SD) in serum samples from mice two days post challenge by qRT-PCR. Values are total RNA copies per reaction, E) Viremia (+/−SD) in serum samples from mice two days post challenge by TCDI₅₀. F) PRNT₅₀ and PRNT₉₀ values (+/−SD) of serum samples taken from ZIKVLP vaccinated AG129 mice post challenge, and pre challenge serum from PBS/alum mice.

FIGS. 3A-B. ZIKVLP serum transfer to naive AG129 mice. A) Average weight loss (+/−SD) of 8 week AG129 transferred serum from mice vaccinated with ZIKVLPs after ID challenge with 20 PFU of ZIKV over a 14 day period. B) Survival of AG129 after challenge with ZIKV over a 14 day period.

FIG. 4. LD50 of ZIKV in AG129 mice. Survival of AG129 after ZIKV over a 14 day period.

FIG. 5A-B. A) Weight loss of AG129 after ID challenge with 20 PFU ZIKV over a 12 day period. B) Survival of AG129 after ID challenge with 200 PFU ZIKV over a 12 day period.

FIG. 6A-B. Sequence of a vector with an exemplary coding sequence to express prM/E (SEQ ID NO:5).

FIG. 7. Schematic of a pCMV pTriex4-neo (B) vector for expression of prM/E.

FIG. 8A-C. Images showing GFP expression in HEK293 cells. A) pTri px4-neo GFP expression, B) pCMV GFP expression, and C) pCMV GFP expression.

FIG. 9. Western blot analysis of pTriex versus pCMV prM/E expression. Lane 1: Zika virus +; lanes 3.9: pCMV-GFP cells (pt.) and supernatant (sup.); lanes 4,10: pCMV-Columbia pt., sup.; lanes 5,11: pCMV-French-Poly pt., sup.; lanes 6, 12: pTriex-Columbia pt., sup.; and lanes 7, 13: pThex-French-Poly pt., sup.

FIG. 10. Anti-Zika antibodies in mice before and after VIP exposure. Mice were injected IP with about 10⁶ TCID₅₀ of ZIKV. 5 weeks later the mice were bled, then injected with crude VLP supernatant. Mice were bled 7 days after injection and antibodies analyzed by ZIKV ELISA.

FIG. 11. Western blot of sucrose purified VLPs. Lane 1: marker; lane 2: VLP 100,000 g precipitation; lane 3: Zika virus +; lane 4: pCMV French-Poly post sucrose purification; and lane 5: pCMV-GFP post sucrose purification. Cells in T-75 flasks were transfected with pCMV-prM/E, or pCMV-GFP, and supernatants were collected after 3 days, then clarified by centrifugation (15,000 g, 30 minutes), then layered onto a 20% sucrose cushion, and pelleted at 112,000 g for 3.5 hours.

FIG. 12. Sucrose fractional analysis. Lane 1: marker; lane 2: Zika virus +; lane 3: Cell debris (pt.) from clarification step; lane 4: Supernatant above sucrose cushion post centrifugation; lane 5: marker; lane 6: VLP post purification batch 1: days 0-3; and lane 7: VLP post purification batch 2: days 3-10. A second batch was harvested from transfected flasks (days 3-10). Purified as before, fractions from each sucrose purification step were analyzed to ensure there was no loss during purification.

FIG. 13. Comparison of protein expression for VLPs produced from pCMV and pTriex constructs.

FIG. 14. Mouse study. 11 AG129 mice of mixed sex and age were used. VLPs were administered IM along with 1 mg Alum. Challenge virus (100 PFU) was administered ID.

FIG. 15. Antibody levels two weeks post boost.

FIG. 16. Survival and morbidity. All controls were moribund on day 9.

FIGS. 17A-C. Dose response of ZIKVLPS in AG129 mice. A-B) PRNT₅₀ and PRNT₉₀ values (+/−SD) of serum samples taken from AG129 mice administered a prime and boost of 0.45 μg (A) or a prime only of 3.0 (B) ZIKVLPs pre and post challenge. C) Survival of 11 week old. AG129 after ID challenge with 200 PFU ZIKV over a 14 day period.

FIGS. 18A-C. Protection of ZIKVLPS in BALB/c mice. A) PRNT₅₀ and PRNT₉₀ values (+/−SD) of serum samples taken from BALB/c mice administered a prime only of 3.0 μg ZIKVLPs post challenge. B) Viremia (+/−SD) in serum samples from mice two days post challenge by qRT-PCR. Values are total RNA copies per reaction. C) Average weight loss (+/−SD) of BALB/c mice after ID challenge with 200 PFU ZIKV over a 14 day period.

### DETAILED DESCRIPTION

### Definitions

As used herein, the terms “isolated” refers to in vitro preparation, isolation of a nucleic acid molecule such as a vector or plasmid of the invention or a virus-like particle of the invention, so that it is not associated with in vivo substances, or is substantially purified from in vitro substances. An isolated virus-like particle preparation is generally obtained by in vitro culture and propagation and is substantially free from infectious agents. As used herein, “substantially free” means below the level of detection for a particular infectious agent using standard detection methods for that agent. As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

A signal peptide (sometimes referred to as signal sequence, secretory signal, e.g., an Oikosin 15 secretory signal, targeting signal, localization signal, localization sequence, transit peptide, leader sequence or leader peptide) is a short (about 5 to 30 amino acids long) peptide present at the N-terminus of proteins that are destined towards the secretory pathway. These proteins include those that reside either inside certain organelles (the endoplasmic reticulum, golgi or endosomes), secreted from the cell, or inserted into most cellular membranes. Although most type I membrane-bound proteins have signal peptides, the majority of type I and multi-spanning membrane-bound proteins are targeted to the secretory pathway by their first transmembrane domain, which biochemically resembles a signal sequence except that it is not cleaved. Signal sequences generally have a tripartite structure, consisting of a hydrophobic care region (h-region) flanked by an n- and c-region. The latter contains the signal peptidase (SPase) consensus cleavage site. Usually, signal sequences are cleaved off co-translationally, the resulting cleaved signal sequences are termed signal peptides.

### Exemplary Embodiments

Zika virus infection transmitted by Aedes mosquitoes is now receiving considerable attention due to its associated with microcephaly and Guillain-Barre syndrome. According to the CDC, there have been over 500 cases of travel-related Zika infections in America to date, with no locally-acquired vector-borne cases reported; in contrast, over 700 cases have been reported in US territories, of which nearly all were locally-transmitted.

Computational analysis has identified ZIKV envelope glycoproteins as a good candidate for vaccine development, as these are the most immunogenic (Shawan, 2015). Several approaches are currently being explored to develop a ZIKV vaccine, including inactivated, recombinant live-attenuated viruses, protein subunit vaccines, or DNA vaccines. A VLP vaccine approach against ZIKV may eliminate concerns of live attenuated vaccines and insufficient inactivation of killed vaccines for pregnant women and other populations at high risk of suffering the devastating effects of ZIKV infections.

VLPs are structurally mimic the conformation of native virions but do not generate progeny viruses (VLPs are “non-infectious”) and do not contain any viral genetic material. VLPs are known to be highly immunogenic and elicit higher titer neutralizing antibody responses than subunit vaccines based on individual proteins (Wang et al., 2013). Such VLPs present viral spikes and other surface components that display linear or conformational epitopes in a repetitive array that, effectively results in recognition by B-cells (Metz and Pijlman, 2016). This recognition leads to B cell signaling and MHC class II up-regulation that facilitates the generation of high titer specific antibodies. VLPs from viruses, including hepatitis B virus, West Nile virus and Chikungunya virus, elicit high titer neutralizing antibody responses that contribute to protective immunity in preclinical animal models and in humans (Akahata et al., 2010; Spohn et al., 2010; Wang et al., 2012).

As mentioned above, a VLP vaccine approach against ZIKV eliminates concerns of live attenuated vaccines and insufficient inactivation of killed vaccines for pregnant women and other populations at high risk of suffering the devastating effects of ZIKV infections. The generation of ZIKV-VLPs containing the prM and E genes as well as the immunogenicity and efficacy testing in the AG129 mouse model is described herein. A position in the secretory signal was identified that likely allows for higher than normal levels of VLP secretion, due to the absence of an auto (NS2b-3) cleavage signal. Using bioinformatic signal sequence prediction tools, the putative signal sequences of ZIKV starting from positions aa 98-aa 112 were examined, and a site was selected that putatively resulted in the highest secretion score. The prM and E genes from ZIKV (Colombian isolate; GenBank accession no. K11646827) were combined with a secretory signal (positions aa 98-aa 112), were cloned into a mammalian expression vector (pCMV-prM/E). HEK-293 cells were transfected and supernatants were harvested from the cells at approximately 10 days post transfection. Transfected HEK-293 cells secreted VLPs with relatively high yields, likely due to the inclusion of a secretory signal that allows for higher than normal levels of VLP secretion. The cell supernatants contained a fraction of extracellular particles that were purified by ultracentrifugation though a sucrose cushion. These particles reacted with known ZIKV antibodies by Western Blot. Western blot analysis also revealed relatively high yields of VLPs after purification, indicating the potential for scalable production. To test the efficacy of this VLP vaccine, AG129 mice susceptible to ZIKV were vacinated with 2 μg of total protein (about 400-500 ng of VLPs) formulated with 1 mg of adjuvant, and the mice boosted with the same vaccine two weeks later. At two weeks post boost, serum from vaccinated animals was collected and tested for anti-ZIKV neutralizing antibodies. Three weeks post boost mice were challenged with 200 PFU of ZIKV (about 400 LD₅₀s). All control animals (n=6) died by 9 days post challenge, while vaccinated mice survived with no morbidity/illness (as of 11 days post-challenge). Passive transfer of antibodies from vaccinated mice was efficacious in protecting susceptible mice from Zika infections. Thus, the present findings show the protective efficacy of a ZIKV-VLP vaccine and highlight the important role that neutralizing antibodies play in protection against ZIKV infection. Further, passive transfer may be employed as a treatment for immune-compromised patients that cannot receive a vaccine.

In one embodiment, a recombinant nucleic acid vector is provided comprising a heterologous promoter operably linked to a sequence encoding ZIKV, prM/E. In one embodiment, the vector lacks nucleic acid sequences encoding ZIKV NS1, NS2A, NS2B, NS3, NS4A, NS4B or NS5 and optionally lacks nucleic acid sequences encoding functional ZIKV capsid, e.g., a protein that aggregates so as to form a viral capsid having a diameter of about 50 to 60 nm. In one embodiment, the heterologous promoter is expressed in mammalian cells. In one embodiment, the heterologous promoter is a heterologous viral promoter. In one embodiment, only a portion of ZIKV capsid sequences is included, a C-terminal portion of a ZIKV capsid that is linked to prM/E sequences as in the polyprotein that is expressed by wild-type flavivirus. In one embodiment, the portion of the capsid sequence includes amino acids 98 to 112 of the capsid protein encoded by SEQ ID NO:1 or a protein having at least 80%, 82%, 85%, 87%, 90%, 92%, 95%, 97%, 99% or more amino acid sequence identity thereto. In one embodiment, the prM/E sequences have at least 80%%, 82%, 85%, 87%, 90%, 92%, 95%, 97%, 99% or more amino acid sequence identity to the prM/E sequences encoded by any one of SEQ ID Nos. 1-3 or 5. In one embodiment, the portion of the capsid sequence lacks a NS2B-3 cleavage site. In one embodiment, the prM/E sequences are operably linked to a heterologous secretion signal. In one embodiment, the vector further comprises an intron and/or enhancer sequence, e.g., 5′ to a prM/E coding sequence.

A recombinant host cell comprising the vector is also provided. In one embodiment, the cell is a mammalian cell. In one embodiment, the cell is a human or simian cell. In one embodiment, the genome of the cell is augmented, e.g., stably augmented, with nucleic acid sequences encoding ZIKV NS2B, e.g., the source of NS2B may be heterologous or homologous to the source for prM/E. In one embodiment, the genome of the cell is augmented, e.g., stably augmented, with nucleic acid sequences encoding ZIKV capsid, e.g., the capsid may he heterologous or homologous to prM/E. In one embodiment, the vector is integrated into the genome of the host cell.

Also provided is a method to prepare ZIKV VLPs. The method includes contacting a culture of isolated host cells that do not express ZIKV NS1, NS2A, NS2B, NS3, NS4A, NS4B or NS5 and optionally do not express functional ZIKV capsid, with the recombinant vector and collecting VLPs from supernatant of the culture. Thus, in one embodiment, the isolated host cells do not have ZIKV sequences prior to contact with the vector. In one embodiment, the collected particles have a diameter of about 10 to 100 nm, e.g., 20 to 60 nm, 40 to 70 nm or 40 to 60 nm. In one embodiment, the host cell expresses ZIKV NS2B. In one embodiment, the host cell expresses ZIKV capsid protein and optionally NS2B.

Further provided is a preparation comprising a ZIKV VLPs. The VLP comprises a lipid bilayer comprising ZIKV prM/E but lacks ZIKV NS1, NS2A, NS2B, NS3, NS4A, NS4B or NS5 and optionally lacks functional ZIKV capsid. Such a preparation may be used in a vaccine or immunogenic composition. The vaccine or immunogenic composition may have about 10 to 1000 μg, e.g., 200 to 400 μg or 400 to 800 μg, or about 1 to about 500 mg, e.g., about 20 to 50 mg, about 100 to 300 or about 300 to 400 mg, of VLP. The vaccine or immunogenic composition may further comprise one or more adjuvants. In one embodiment, an adjuvant is included at about 0,01 to about 10 mg, about 1 to about 20 mg, or about 10 mg to about 100 mg.

Further provided is a method to prevent, inhibit or treat ZIKV infection in a mammal. The method includes administering an effective amount of the recombinant vector, a host cell having the vector or the vaccine or immunogenic composition having the VLPs. In one embodiment, the mammal is a female mammal. In one embodiment, the vector, host cell, vaccine or immunogenic composition is administered intradermally, intramuscularly or intravenously to the mammal.

In one embodiment, a method to passively prevent, inhibit or treat ZIKV infection in a mammal is provided. The method includes obtaining serum or plasma having anti-ZIKV antibodies from a mammal exposed to ZIKV and optionally isolating antibodies from the serum or plasma; and administering an effective amount of the serum or plasma, or isolated antibodies, to a different mammal at risk of or having a ZIKV infection. In one embodiment, the mammal is immunocompromised. In one embodiment, the anti-flavivirus antibodies are isolated from the serum before administration. In one embodiment, the mammal is a human.

### Exemplary Adjuvants

Adjuvants are compounds that enhance the specific immune response against co-inoculated antigens. Adjuvants can be used for various purposes: to enhance the immunogenicity of highly purified or recombinant antigens; to reduce the amount of antigen or the number of immunizations needed for protective immunity; to prime the efficacy of vaccines in newborns, the elderly or immuno-compromised persons; or as antigen delivery systems for the uptake of antigens by the mucosa. Ideally, adjuvants should not induce immune responses against themselves and promote an appropriate immune response (i.e., cellular or antibody immunity depending on requirements for protection). Adjuvants can be classified into three groups: active immunostimulants, being substances that increase the immune response to the antigen; carriers being immunogenic proteins that provide T-cell help; and vehicle adjuvants, being oil emulsions or liposomes that serve as a matrix for antigens as well as stimulating the immune response.

Adjuvant groups include but are not limited to mineral salt adjuvants, e.g., alum-based adjuvants and salts of calcium, iron and zirconium; tensoactive adjuvants, e.g., Quil A which is a saponin derived from an aqueous extract from the bark of Quillaja sapanaria: Saponins induce a strong adjuvant effect to T-dependent as well as T-independent antigens. Other adjuvant groups are bacteria-derived substances including cell wall peptidoglycan or lipopolysaccharide of Gram-negative bacteria, that enhance immune response against co-administered antigens and which is mediated through activation of Toll-like receptors; lipopolysaccharides (LPS) which are potent B-cell mitogens, but also activate T cells; and trehalose dimycolate (TCM), which simulates both humoral and cellular responses.

Other adjuvants are emulsions, e.g., oil in water or water in oil emulsions such as FIA (Freund's incomplete adjuvant), Montanide, Adjuvant 65, and Lipovant; liposomes, which may enhance both humoral and cellular immunity; polymeric adjuvants such as biocompatible and biodegradable microspheres; cytokines; carbohydrates; inulin-derived adjuvants, e.g., gamma inulin, a carbohydrate derived from plant roots of the Compositae family, is a potent humoral and cellular immune adjuvant and algammulin, which is a combination of γ-inulin and aluminium hydroxide. Other carbohydrate adjuvants include polysaccharides based on glucose and mannose including but not limited to glucans, dextrans, lentinans, glucomannans, galactomannans, levans and xylans.

Some well known parenteral adjuvants, like MDP, monophosphoryl lipid A (MPL) and LPS, also act as mucosal adjuvants. Other mucosal adjuvants poly(DL-lactide-coglycolide) (DL-PLG), cellulose acetate, iminocarbonates, proteinoid microspheres, polyanhydrides, dextrans, as well as particles produced from natural materials like alginates, geletine and plant seeds.

Adjuvants for DNA immunizations include different cytokines, polylactic microspheres, polycarbonates and polystyrene particles.

In one embodiment, adjuvants useful in the vaccines, compositions and methods described herein include, but are not limited to, mineral salts such as aluminum salts, calcium salts, iron salts, and circonium slats, saponin, e.g., Quid A including QS21, squalene (e.g., AS03), TLR ligands, bacterial MDP (N-acetyl muramyl-L-alanyl-D-isoglutamine), lipopolysaccharide (LPS), Lipid A, montanide, Adjuvant 65, Lipovant, Incomplete Freund's adjuvant (IFA), liposmes, microparticles formed of, for example, poly(D,L-lactide (coglycolide)), cytokines, e.g., IFN-gamma or GMCSF, or carbohydrates such as gamma inulin, glucans, dextrans, lentinans, glucomannans and/or glactomannans.

### Pharmaceutical Compositions

Pharmaceutical compositions of the present invention, suitable for inoculation or for parenteral or oral administration, comprise flavivirus VLPs, optionally further comprising sterile aqueous or non-aqueous solutions, suspensions, and emulsions. The compositions can further comprise auxiliary agents or excipients, as known in the art. See, e.g., Berkow et al., 1987: Avery's Drug Treatment, 1987. The composition of the invention is generally presented in the form of individual doses (unit doses).

Vaccines may contain about 0.1 to 500 ng, 0.1 to 500 μg, or 1 to 100 μg, of VLPs. In one embodiment, the vaccine may contain about 100 μg to about 500 μg of VLPs. In one embodiment, the vaccine may contain about at least 100 ng of VLPs. In one embodiment, the vaccine may contain about at least 500 ng of VLPs. In one embodiment, the vaccine may contain about at least 1000 ng of VLPs. In one embodiment, the vaccine may contain about at least 50 μg of VLPs, In one embodiment, the vaccine may contain less than about 750 μg of VLPs. In one embodiment, the vaccine may contain less than about 250 μg of VLPs. In one embodiment, the vaccine may contain less than about 100 μg of VLPs. In one embodiment, the vaccine may contain less than about 40 μg of VLPs. The vaccine forming the main constituent of the vaccine composition of the invention may comprise a combination of different flavirus VLPs, for example, at least two of the three types, Chinese, West African or East African.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Avery's, 1987.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized. Examples of materials suitable for use in vaccine compositions are provided.

A pharmaceutical composition according to the present invention may further or additionally comprise at least one chemotherapeutic compound, for example, immunosuppressants, anti-inflammatory agents or immune enhancers, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines, are provided before any symptom of a pathogen infection becomes manifest. The prophylactic administration of the composition serves to prevent or attenuate any subsequent infection or one or more symptoms associated with the disease.

When provided therapeutically, a VLP vaccine is provided upon the detection of a symptom of actual infection. The therapeutic administration of the vaccine serves to attenuate any actual infection. See, e.g., Avery, 1987.

Thus, a VLP vaccine composition of the present invention may thus be provided either before the onset of infection (so as to prevent or attenuate an anticipated infection) or after the initiation of an actual infection.

A composition is said to be “pharmacologically acceptable” if its administration can be tolerated by a recipient patient. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious flavivirus.

The “protection” provided need not he absolute, i.e., the flavivirus infection need not be totally prevented or eradicated, if there is a statistically significant improvement compared with a control population or set of patients. Protection may be limited to mitigating the severity or rapidity of onset of symptoms of the flavivirus infection.

### Pharmaceutical Administration

A composition of the present invention may confer resistance to one or more pathogens, e.g., one or more flavivirus strains, by either passive immunization or active immunization. In active immunization, an inactivated or attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one flavivirus strain.

In one embodiment, the vaccine or immune serum is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present invention thus includes methods for preventing or attenuating a disorder or disease, e.g., an infection. As used herein, a vaccine is said to prevent or attenuate an infection if its administration results either in the total or partial attenuation (i.e., suppression) of a symptom or condition of the infection, or in true total or partial immunity of the individual to the disease.

At least one VLP or composition thereof, of the present invention may be administered by any means that achieve the intended purposes, using a pharmaceutical composition as previously described.

For example, administration of such a composition may be by various parenteral routes such as subcutaneous, intravenous, intradermal, intramuscular, intraperitoneal, intranasal, oral or transdermal routes. Parenteral administration can be by bolus injection or by gradual perfusion over time. One mode of using a pharmaceutical composition of the present invention is by intramuscular or subcutaneous application. See, e.g., Avery, 1987.

A typical regimen for preventing, suppressing, or treating a flavivirus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent suggested dose ranges. However, the dosage will he tailored to the individual subject, as is understood and determinable by one of skill in the art. See, e.g., Avery's, 1987; and Ebadi, 1985.

The invention will be further described by the following non-limiting examples.

### EXAMPLE 1

### Experimental Procedures

### Cells and Viruses

African Green Monkey kidney cells (Vero) and Human embryonic kidney 293 (HEK293) were obtained from ATCC (ATCC; Manassas, Va., USA) and grown in Dulbecco's modified Eagle medium (DMEM) supplemented with 10% fetal bovine serum (FBS; Hyclone, Logan, Utah), 2 mM L-glutamine, 1.5 g/L sodium bicarbonate, 100 U/mL of penicillin, 100 μg/mL of streptomycin, and incubated at 37° C. in 5% CO₂. ZIKV strain H/PF/2013 (GenBank:KJ776791), was obtained from Xavier de Lamballerie (European Virus Archive, Marseille France). Virus stocks were prepared by inoculation onto a confluent monolayer of Vero cells.

### Animals

Mice of the 129/Sv background deficient in alpha/beta interferon (IFN-α/β) and IFN-γ receptors (AG129 mice) were obtained from B&K Universal Limited (Hull, England) and were bred in the pathogen-free animal facilities of the University of Wisconsin-Madison School of Veterinary Medicine. Groups of mixed sex mice were used for all experiments.

Production and purification of ZIKV VLPs

The prM and E genes of ZIKV strain H/PF/2013 with nascent signal sequence were cloned into a pCMV expression vector under the control of a cytomegalovirus (CMV) promoter and CMV polyadenylation signal (pCMV-prM/E). Endotoxin free, transfection grade DNA was prepared using Maxiprep kit (Zymo Research, Irvine, Calif.). VLPs were expressed by transfecting 90% confluent monolayers of HEK293 cells in a T-75 flasks with 15 μg of pCMV-prM/E using Eugene HD (Promega, Madison, Wis.) transfection reagent according to manufacturer protocol. The 10 ml supernatant was harvested 72 hours after transfection, and. clarified by centrifugation at 15,000 RCF for 30 minutes at 4° C. Clarified supernatants were layered onto a 20% sucrose cushion and ultra-centrifuged in a SW-28 rotor at 112,000 RCF for 3.5 hours at 4° C. Pellet (PT) and supernatant (SUP) fractions at each step were saved for analysis by SDS-PAGE and Western blot. Post sucrose cushion PT were resuspended in Phosphate Buffered. Saline (PBS) pH 7.2. Total protein in VLP preparations was quantified by Bradford assay. VLP specific protein was determined by comparing Zika specific bands on SDS-PAGE gels to known concentrations of BSA using ImageJ software.

### Western Blot

VLP fractions were boiled in sample buffer (BioRad, Hercules, Calif., USA) and resolved on a 4-20% SDS-PAGE gel (Biorad) by electrophoresis using a Mini-PROTEAN 3 system (RIO-RAD, Calif.). Gels were electroblotted onto nitrocellulose membranes using a Turboblot® system. Membranes were blocked in 5% (W/V) skim milk and probed with mouse hyper immune ascites fluid primary antibody (1:5000) and goat anti-mouse HRP conjugated secondary antibody (1:5000). Membranes were developed using a solid phase 3,3′,5,5′-tetramethylbenzidine (TMB) substrate system.

### Transmission Electron Microscopy

Samples were negatively stained for electron microscopy using the drop method. A drop of sample was placed on a Pioloform™ (Ted Pella, Inc.) carbon-coated 300 Mesh Cu grid, allowed to adsorb for 30 seconds, and the excess removed with filter paper. Next, a drop of methylamine tungstate or uranyl acetate (Nano-W, Nanoprobes Inc.) was placed on the still wet grid, and the excess removed. The negatively stained sample was allowed to dry, and was documented in a Philips CM120(Eindhoven, The Netherlands) transmission electron microscope at 80 kN. Images were obtained using a SIS MegaView III digital camera (Soft Imaging Systems, Lakewood, Colo.).

### Vaccination and Viral Challenge

For VLP formulations, 0.45 μg of sucrose cushion purified. VLPs was mixed with 0.2% inject Alum (Thermo Scientific) according to manufacturer's protocol. Groups of AG129 mice were injected intramuscularly (IM) with VLPs mixed with alum (n=5) or PBS mixed with alum (n=6) at 6 weeks of age, and again at 8 weeks of age. Sub-mandibular blood draws were performed pre boost and pre challenge to collect serum for analysis by neutralization assays and for passive transfer studies.

Vaccinated mice were challenged with 200 PFU of ZIKV strain H/PF/2013 in 25 μl volumes by intradermal (ID) injection into the right hind footpad. Following infection, mice were monitored daily for the duration of the study. Mice that were moribund or that lost greater than 20% of starting weight were humanely euthanized. Sub-mandibular blood draws were performed on day two post challenge (PC) and serum collected to measure viremia.

For passive transfer studies, 5 naive mice were injected intraperitoneally (IP) with 500 μl of pooled serum from VLP vaccinated, diluted serum (1:5 n=4, 1:10, n=4), or serum from PBS/alum (n=5) treated mice. At 12 hours post transfer, mice were challenged with 20 PFU in 2.5 μl as above.

### Viremia Assays

Viremia was determined by TCIDSO assay. Briefly, serum was serially diluted ten-fold in microtiter plates 263 and added to duplicate wells of Vero cells in 96-well plates, incubated at 37° C. for 5 days, then fixed and 264 stained with 10% (W/V) crystal violet in 10% (V/V) formalin. Plates were observed under a light microscope to determine the 50% tissue culture infective doses (TCID50s). Serum samples were also tested for viral RNA copies by qRT-PCR. RNA was extracted from 0.02 ml of serum using the ZR Viral 267 RNA Kit (Zymo Research, Irvine, Calif.). Viral RNA was quantified by qRT-PCR using the primers and probe designed by Lanciotti et al. (Lanciotti et al., 2008). The qRT-PCR was performed using the iTaq Universal Probes One-Step Kit (BioRad, Hercules, Calif.) on an iCycler instrument (BioRad, Hercules, Calif.). Primers and probe were used at final concentrations of 500 nM and 250 nM respectively. Cycling conditions were as follows: 50° C. for 10 minutes and 95° C. for 2 minutes, followed by 40 cycles of 95° C. for 15 seconds and 60° C. for 30 seconds. Virus concentration was determined by interpolation onto an internal standard curve made up of a 5-point dilution series of in vitro transcribed RNA.

### Neutralization Assay

Serum antibody titers were determined by microneutralization assay. Briefly, serum was incubated at 56° C. for 30 minutes to inactivate complement and then serially diluted two-fold in microtiter plates. 200 PFUs of virus were added to each well and incubated at 37° C. for 1 hour. The virus-serum mixture was added to duplicate wells of Vero cells in 96-well plates, incubated at 37° C. for 5 days, then fixed and stained with 10% (W/V) crystal violet in 10% (WV) formalin, then observed under a light microscope. The titer was determined as the serum dilution resulting in the complete neutralization of the virus.

### Plaque Reduction Neutralization Test

Serum samples were serially diluted, mixed with 200 PFU of the ZIKV H/PF/2013 strain and incubated for 1 hour at 37° C. This serum/virus mixture was added to confluent layers of Vero cells in 96 well plates and incubated for 1 hour at 37° C., after which the serum/virus mixture was removed and overlay solution (3% CMC, 1× DMEM, 2% FBS and 1× Anti/Anti) was added. After 48 hours of infection, the monolayers were fixed with 4% PFA, washed twice with PBS, and then incubated with ZIKV hyperimmune mouse ascitic fluid (1:2000, UTMB) diluted in blocking solution (1× PBS, 0.01% Tween-20 and 5% Milk) and incubated. overnight at 4° C. Plates were washed three times with PBS-T and then peroxidase-labeled goat anti-mouse secondary antibody (1:2000) was incubated on monolayers for 2 hours at 37° C. Following incubation, cells were washed a final three times with PBS-T and developed using 3-amino-9-ethylcarbazole (AEC)-peroxidase substrate. The amount of formed foci were counted using an 292 ELISPOT plate reader (ImmunoSPOT-Cellular Technology); quality control was performed to each scanned well to ensure accurate counting. Neutralization percentages (NA) were calculated per sample/replicate/dilution as follows:

$$Nx \{ 100 - [ 100 ( \frac{A}{Control} )$$

Where A corresponds to the amount of foci counted in the sample and Control is the geometric mean of foci counted from wells treated with cells and virus only. Data of corresponding transformed dilutions (Log(1/Diltition)) against neutralization percentages per sample was plotted and fitted to a sigmoidal dose-299 response curve to interpolate PRNT₅₀ and PRNT₉₀ values (GraphPad Prism software).

SEQ ID NO: 1:
mknpkkksgg frivnmlkrg varvspfggl krlpaglllg hgpirmvlai laflrftaik
pslglinrwg svgkkeamei ikkfkkdlaa mlriinarke kkrrgadtsv givgllltta
maaevtrrgs ayymyldrnd ageaisfptt lgmnkcyiqi mdlghmcdat msyecpmlde
gvepddvdcw cnttstwvvy gtchhkkgea rrsrravtlp shstrklqtr sqtwlesrey
tkhlirvenw ifrnpgfala aaaiawllgs stsqkviylv milliapays ircigvsnrd
fvegmsggtw vdvvlehggc vtvmaqdkpt vdielvtttv snmaevrsyc yeasisdmas
dsrcptqgea yldkqsdtqy vckrtlvdrg wghgcglfgk gslvtcakfa cskkmtgksi
gpenleyrim lsvhgsqhsg mivndtghet denrakveit pnspraeatl ggfgslgldc
eprtgldfsd lyyltmnnkh wlvhkewfhd iplpwhagad tgtphwnnke alvefkdaha
krqtvvvlgs qegavhtala galeaemdga kgrlssghlk crlkmdklrl kgvsyslcta
aftftkipae tlhgtvtvev qyagtdgpck vpaqmavdmq tltpvgrlit anpviteste
nskmmleldp pfgdsyivig vgekkithhw hrsgstigka featvrgakr mavlgdtawd
fgsvggalns lgkgihqifg aafkslfggm swfsqiligt llmwlglntk ngsislmcla
lggvliflst avsadvghsv dfskketrcg tgvfvyndve awrdrykyhp dsprrlaaav
kqamedgicg issvsrmeni mwrsvegeln aileengvql tvvvgsvkhp mwrgpqrlpv
pvnelphgwk awgksyfvra aktnnsfvvd gdtlkecplk hrawnsflve dhgfgvfhts
vwlkvredys lecdpavigt avkgkeavhs dlgvwiesek ndtwrlkrah liemktcewp
kshtlwtdgi eesdliipks lagplshhht regyrtqmkg pwhseeleir feecpgtkvh
veetcgtrgp slrsttasgr vieewccrec tmpplsfrak dgcwygmeir prkepesnlv
rsmvtagstd hmdhfslgvl villmvqegl kkrmttkiii stsmavlvam ilggfsmsdl
aklailmgat faemntggdv ahlaliaafk vrpallvsfi franwtpres mllalascll
qtaisalegd lmvlingfal awlairamvv prtdnitlai laaltplarg tllvawragl
atcggfmlls lkgkgsvkkh lpfvmalglt avrlvdpinv vglllltrsg krswppsevl
tavglicala ggfakadiem agpmaavgll ivsyvvsgks vdmyieragd itwekdaevt
gnsprldval desgdfslve ddgppmreii lkvvlmticg mnpiaipfaa gawyvyvktg
krsgalwdvp apkevkkget tdgvyrvmtr rllgstqvgv gvmgegvfht mwhvtkgsal
rsgegrldpy wgdvkqdlvs ycgpwkldaa wdghsevqll avppgerarn iqtlpgifkt
kdgdigaval dypagtsgsp ildkcgrvig lygngvvikn gsyvsaitqg rreeetpvec
fepsmlkkkq ltvldlhpga gktrrvlpei vreaiktrlr tvilaptrvv aaemeealrg
lpvrymttav nvthsgteiv dlmchatfts rllqpirvpn ynlyimdeah ftdpssiaar
gyistrvemg eaaaifmtat ppgtrdafpd snspimdtev evperawssg fdwvtdhsgk
tvwfvpsvrn gneiaacltk agkrviqlsr ktfetefqkt khgewdfvvt tdisemganf
kadrvidsrr clkpvildge rvilagpmpv thasaaqrrg rigrnpnkpg deylvgggca
etdedhahwl earmlldniy lqdgliasly rpeadkvaai egefklrteq rktfvelmkr
gdlpvwlayq vasagitytd rrwcfdgttn ntimedsvpa evwtrhgekr vlkprwmdar
vcsdhaalks fkefaagkrg aafgvmealg tlpghmterf qeaidnlavl mraetgsrpy
kaaaaqlpet letimllgll gtvslgiffv lmrnkgigkm gfgmvtlgas awlmwlseie
pariacvliv vflllvvlip epekqrspqd nqmaiiimva vgllglitan elgwlertks
dlshlmgrre egatigfsmd idltpasawa iyaalttfit pavqhavtts ynnyslmama
tgagvlfgmg kgmpfyawdf gvpllmigcy sgltpltliv aiillvahym ylipglqaaa
araaqkrtaa gimknpvvdg ivvtdidtmt idpqvekkmg qvlliavavs sailsrtawg
wgeagalita atstlwegsp nkywnsstat slcnifrgsy lagasliytv trnaglvkrr
gggtgetlge kwkarlnqms alefysykks gitevcreea rralkdgvat gghavsrgsa
klrwlvergy lqpygkvidl gcgrggwsyy aatirkvqev kgytkggpgh eepmlvqsyg
wnivrlksgv dvfhmaaepc dtllcdiges ssspeveear tlrvlsmvgd wlekrpgafc
ikvlcpytst mmetlerlqr ryggglvrvp lsrnsthemy wvsgaksnti ksysttsqll
lgtmdgprrp vkyeedvnlg sgtravvsca eapnmkiigh rierirseha etwffdenhp
yrtwavhgsy eaptqgsass lingvvrlls kpwdvvtgvt giamtdttpy gqqrvfkekv
dtrypdpqeg trqvmsmvss wlwkelgkhk rprvctkeef inkvrsnaal gaifeeekew
ktaveavndp rfwalvdker ehhlrgecqs cvynmmgkre kkqgefgkak gsraiwymwl
garflefeal gflnedhwmg rensgggveg lglqrigyvl eemsripggr myaddtagwd
trisrfdlen ealitnqmek ghralalaii kytyqnkvvk vlrpaekgkt vmdiisrqdq
rgsgqvvtya lntftnlvvg lirnmeaeev lemgdlwllr rsekvtnwlq sngwdrlkrm
avsgddcvvk piddrfahal rflndmgkvr kdtqewkpst gwdnweevpf cshhfnklhl
kdgrsivvpc rhqdeligra rvspgagasi retaclaksy aqmwqllyfh rrdlrlmana
icssvpvdwv ptgrttwsih gkgewmtted mlvvwnrvwi eendhmedkt pvtkwtdipy
lgkredlwcg slighrprtt waenikntvn mvrriigdee kymdylstqv rylgeegstp
gvl

### Results

### Expression and Purification of Soluble, Zika VLPs

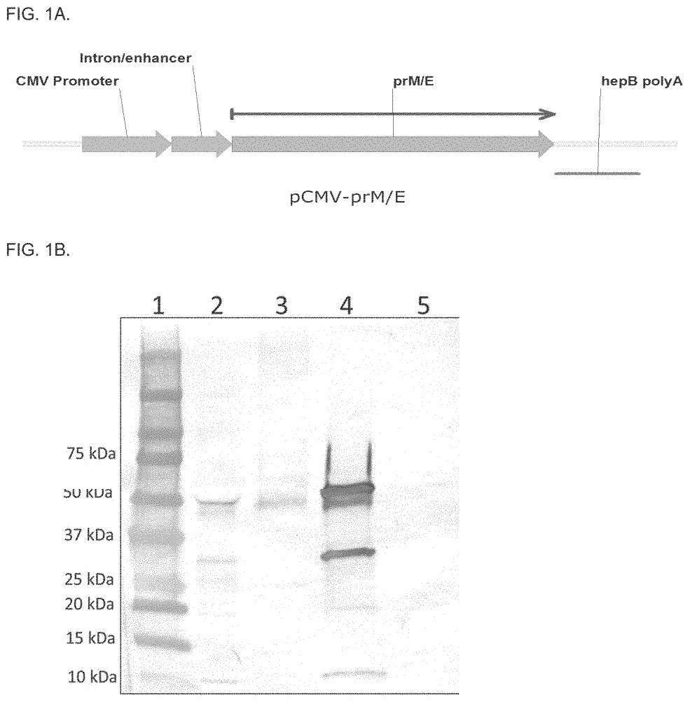

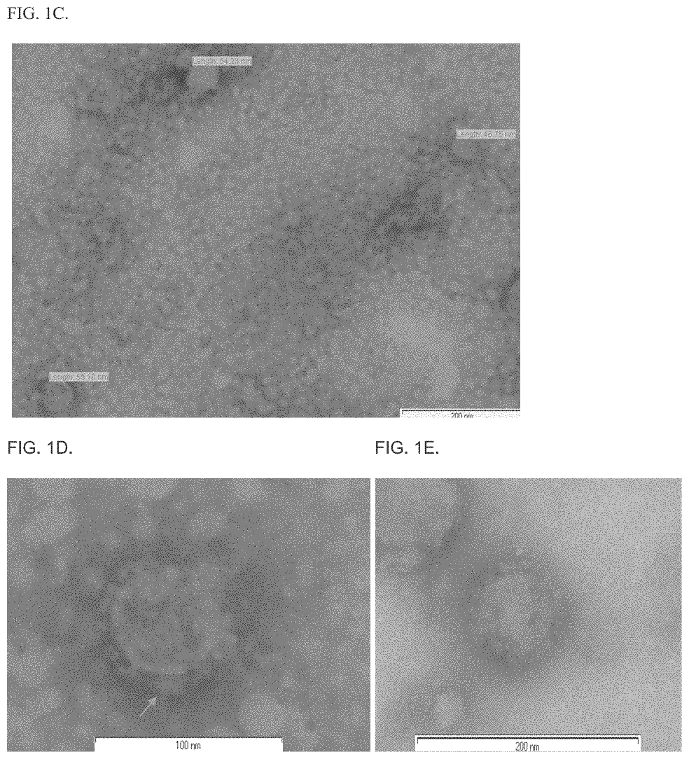

To generate Zika VLPs (ZIKVLPs), the prM/E genes with a native signal sequence were cloned into a pCMV expression vector (pCMV-prM/E) (FIG. 1A), transfected HEK293 cells and harvested supernatants (supe) 3 days post transfection. 78 μg total protein was recovered from post sucrose purification of which 21.6 μg was VLP protein. Western blot analysis of this pCMV-prM/E supe. revealed expression of about 50 kDa size band (FIG. 1B, lane 2) that corresponded in size to the predicted size of the Zika viers E gene, and additionally matched positive control Zika virus stocks (FIG. 1B, lane 3). To test the hypothesis that expression of Zika prM and E genes spontaneously form extracellular particles, supernatants from pCMV-prM/E and pCMV-GFP (negative control) transfected cells were centrifuged on a sucrose cushion (SC) sufficient for pelleting of flavi virus particles from cell culture proteins (Merino-Ramos et al., 2014). pCMV-prM/E SC purified pellet (pt) appeared to contain high levels of E protein, while pCMV-GFP pt. did not, indicating that staining was specific to expression of 100 prM and E genes.

To determine if the immune reactive extracellular particles were virus like in nature, transmission electron microscopy (TEM) was performed on pCMV-prM/E SC pt. material. TEM revealed flavi virus 103 like particles with a size that ranged from 30-60 nm (data not show), and a typical size of about 50 nm (FIG. 1C). High magnification images demonstrated surface structures characteristic of flaviral envelope proteins (FIGS. 1D, E).

### Administration of ZIKVLPs is Immunogenic and Protects Highly ZIKV Susceptible α/β/γ Interferon Deficient Mice

Mice that received ZIKVLPs developed low levels (GMT=1:9.2) of neutralizing antibodies (nAbs) at 109 two weeks post administration, that increased two weeks after boost (GMT=1:32). Five weeks after primary vaccination, all mice were challenged with 200 PFU of ZIKV by the ID route. Mice administered ZIKVLP maintained weight, while mice that received PBS/alum experienced significant weight loss associated morbidity throughout the challenge period.

All control mice (n=6) died 9 days after ZJKV challenge. Mice administered ZIKVLP survived with no apparent morbidity. Finally, ZIKVLP vaccinated mice had significantly lower levels of viremia on day 2 post challenge than control mice detected by qRT-PCR (p=0.0356) and 116 TCID50 assay (p=0.0493).

ZIKVLPs Elicit Plaque Reducing Neutralizing Antibody Titers in Mice that can be Passively Transferred to Naïve Mice.

The plaque reduction neutralization test (PRNT) assay is widely considered to be the “gold standard” for characterizing and quantifying circulating levels of anti-dengue and other flaviviral neutralizing antibodies (nAb) (Thomas et al., 2009). A PRNT assay was developed for rapidly measuring ZIKV specific neutralizing antibodies. Pooled serum samples collected from mice pre-challenge, as well as individual serum samples collected from mice post-challenge were tested by this PRNT assay. Pre-challenge, pooled serum from mice administered ZIKVLP had a calculated 90% plaque reduction (PRNT₉₀) titer of 1:34. The PRNT₉₀ titer increased 2 weeks post challenge (GMT=126 662).

To test the role of anti-ZIKV antibodies in protection against challenge, groups of mice received ZIKVLP 128 antiserum, undiluted (n=5), diluted 1:5 (n=4), or 1:10 (n=4). As a negative control, mice (n=5) were transferred serum from mice previously vaccinated with PBS alum.

Negative control mice rapidly lost weight starting after day 7 and all died day 9 post challenge. Mice that received undiluted serum maintained weight throughout the 12 day period post challenge, and showed no signs of infection. Mice that received diluted anti-ZIKV antibodies were not protected from challenge, although survival and weigh loss were slightly extended relative to negative control mice 134.

### Discussion

Most experts and public health workers agree that a Zika vaccine is urgently needed. In February 2016, the World Health Organization declared that the recent clusters of microcephaly and other neurological disorders in Brazil constitute a public health emergency of international concern. Their recommendations included enhanced surveillance and research, as well as aggressive measures to reduce infection with Zika virus, particularly amongst pregnant women and women of childbearing age. ZIKV is now receiving considerable attention due to its rapid spread in the Americas, and its association with microcephaly (Mlakar et al., 2016) and Guillain-Barre syndrome (Pinto Junior et al., 2015). In our studies, we designed a ZIKV-virus-like particle (VLP) vaccine, demonstrated expression in vitro by western blot and transmission electron microscopy, and tested the protective efficacy and role of antibodies in protection in the AG129 mouse model.

Although the transfection and purification procedures for this ZIKV-VLP have yet to be optimized, we had an overall calculated yield of 2.2 mg/ml. Similar expression levels have been reported for other flavivirus VLP expression strategies (Pijlman, 2015). Future work will optimize VLP production and purification parameters, which should significantly increase both yield and purity. Stably transfected. HEK cells that continuously express VLPs allow for scalable production to meet global demand for a ZIKV vaccine.

ZIKV-VLPs, formulated with alum, induced detectable neutralizing antibodies and protected animals against lethal challenge (>400 LD50s) with no morbidity or weight loss. Pre-challenge GMT neutralizing titers were 1:32, and pooled pre-challenge serum PRNT₉₀ and PRNT₅₀ titers were 1:34 and 1:157 respectively. At a relatively low dose of 450 ng, the present results indicate that the ZIKV VLPs are highly immunogenic. Additionally, the antibody titers we obtained are consistent with those reported for other highly immunogenic flavivirus VLP vaccines (Ohtaki et al., 2010; Pijiman. 2015).

Vaccinated mice challenged with >400 LD50s had low levels of viremia (mean=127, geometric mean=25.4 TCID50/ml) detected after challenge. Copies of RNA ZIKV genomes in serum of mice were significantly higher than levels of viremia. However, the disparity between viral genome copies and viremia has been observed for other flaviviruses including dengue (Bae et al., 2003). Since AG129 mice are highly susceptible to viral challenge, it is possible that the challenge dose given for the active vaccination study was artificially high. Additionally, methods for challenging mice from infected mosquito bite should be developed to most accurately mimic natural infection. Animal studies can determine if the ZIKVLP vaccine can protect female mice from contracting ZIKV during pregnancy using established models for such studies (Miner et al., 2016). ZIK-VLP vaccines may be tested in a non-human primate translational model which most accurately mimics human infection.

A VLP vaccine approach against ZIKV has significant advantages over other technologies as it will eliminate concerns of live attenuated vaccines and insufficient inactivation of killed vaccines for pregnant women and other populations at high risk of suffering the devastating effects of ZIKV infections. In recent years, recombinant virus-like particle (VLF)-based vaccine strategies have been frequently used for novel vaccine design. VLPs are known to be highly immunogenic and elicit higher titer neutralizing antibody responses than subunit vaccines based on individual proteins (Ariano et al., 2010).

The role of neutralizing antibodies in protecting against ZIKV was demonstrated by antibody passive transfer studies as naive AG129 mice receiving pooled serum from VLP vaccinated animals were fully protected. These results are consistent with previous findings that indicate the important role of antibodies in protecting against many mosquito-borne viruses, such as Japanese encephalitis, yellow fever and chikungunya. In this study, full protection was observed when animals received undiluted serum, with no weight loss or other clinical signs observed. While these studies highlight the importance of serum antibodies in ZIKV protection, upcoming studies will determine the minimum antibody titer needed for protection, whether the ZIKV-VLP can elicit CD8+ responses, and the overall role of cellular immunity in protection. It is also important to determine whether anti-ZIKV antibodies elicited by the VLPs play any role in dengue protection or disease enhancement.

In this study, the AG129 IFN receptor-deficient mouse model was used for evaluation of the ZIKV-VLP. Recently, the suitability of mice deficient in IFN-α/β and -γ receptors as an animal model for ZIKV was demonstrated, as they are highly susceptible to ZIKV infection and disease, developing rapid viremic dissemination in visceral organs and brain and dying 7-8 days post-infection (Aliota et al., 2016). The AG129 mouse model exhibits an intact adaptive immune system, despite the lack of an IFN response, and it has been used extensively to evaluate vaccines and antivirals for DENV (Brewoo et al., 2012; Fuchs et al., 2014; Johnson and Roehrig, 1999; Sarathy et al., 2015).

In the present study, aluminum hydroxide (commonly known as alum) was used as the adjuvant for the ZIKV-VLP preparations. Since its first use in 1932, vaccines containing aluminum-based adjuvants have been successfully administered. in humans demonstrating excellent safety. A variety of adjuvant formulations may, however, be employed with ZIKV VLPs to enhance immunogenic potential including adjuvants that facilitate antigen dose sparing, enhanced immunogenicity, and/or broadened pathogen protection.

Thus, a VLP based Zika vaccine is described herein that elicits protective antibodies in mice, and is safe, suitable for scalable production, and highly immunogenic. Fast-tracking development of this ZIKV vaccine is a public health priority and is crucial for restoring confidence and security to people who wish to have children or reside in, or visit areas in which ZIKV is endemic.

### EXAMPLE 2

Exemplary Zika virus polyprotein sequences:
Accession No. KU646827 (which is incorporated by reference herein)

(SEQ ID NO: 6)
IRCIGNTSNRETVEGMSGGTWVDVVLEHGGCVTVMAQDKPTVDIE
LVTTTVSNMAEVRSYCYEASISDMASDSRCPTQGEAYLDKQSDTQYVCKRTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLDCEPRTGLDFSDLYYLTMNNKHWLVHKEWF
HDIPLPWHAGADTGTPHWNNKEALVEFKDAHAKRQTVVVLGSQEGAVHTALAGALEAE
MDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVG
EKKITHHNVHRSGSTIGKAFEATVRGAKRMAVLGTAWDFGSVGGALNSLGKGIHQIFG
AAFKSLFGGMSWFSQILIGTLLMWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSVKNPMWRGPQRLPVPVNELPHGWKAWGK
SYFVRAAKTNNSFVVDGDTLKECPLKHRAWNSFLVEDHGFGVFHTSVWLKVREDYSLE
CDPAVIGTAVKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFWAKDGCWYGMEIRPRKEPESNLVRSMVTA
GSTDHMDHFSL
(SEQ ID NO: 1)
atacggtgca taggagtcag caatagggac tttgtggaag gtatgtcagg tgggacttgg
gttgatgtcg tcttggaaca tggaggttgt gtcaccgtaa tggcacagga caaaccgact
gtcgacatag agctggttac aacaacagtc agcaacatgg cggaggtaag atcctactgc
tatgaggcat caatatcaga catggcttcg gacagccgct gcccaacaca aggtgaagcc
taccttgaca agcaatcaga cactcaatat gtctgcaaaa gaacgttagt ggacagaggc
tggggaaatg gatgtggact ttttggcaaa gggagcctgg tgacatgcgc taagtttgca
tgctccaaga aaatgaccgg gaagagcatc cagccagaga atctggagta ccggataatg
ttgtcagttc atggctccca gcacagtggg atgatcgtta atgacacagg acatgaaact
gatgagaata gagcgaaggt tgagataacg cccaattcac caagagccga agccaccctg
gggggttttg gaagcctagg acttgattgt gaaccgagga caggccttga cttttcagat
ttgtattact tgactatgaa taacaagcac tggttggttc acaaggagtg gttccacgac
attccattac cttggcacgc tggggcagac accggaactc cacactggaa caacaaagaa
gcactggtag agttcaagga cgcacatgcc aaaaggcaaa ctgtcgtggt tctagggagt
caggaaggag cagttcacac ggcccttgct ggagctctgg aggctgagat ggatggtgca
aagggaaggc tgtcctctgg ccacttgaaa tgtcgcctga aaatggacaa acttagattg
aagggcgtgt catactcctt gtgtaccgca gcgttcacat tcaccaagat cccggctgaa
acactgcacg ggacagtcac agtggaggta cagtacgcag ggacagatgg accttgcaag
gttccagctc agatggcggt ggacatgcaa actctgaccc cagttgggag gttgataacc
gctaaccccg taatcactga aagcactgag aactctaaga tgatgctgga acttgatcca
ccatttgggg actcttacat tgtcatagga gtcggggaga agaagatcac ccaccactgg
cacaggagtg gcagcaccat tggaaaagca tttgaagcca ctgtgagagg tgccaagaga
atggcagtct tgggagacac agcctgggac tttggatcag ttggaggcgc tctcaactca
ttgggcaagg gcatccatca aatttttgga gcagctttca aatcattgtt tggaggaatg
tcctggttct cacaaattct cattggaacg ttgctgatgt ggttgggtct gaacacaaag
aatggatcta tttcccttat gtgcttggcc ttagggggag tgttgatctt cttatccaca
gccgtctctg ctgatgtggg gtgctcggtg gacttctcaa agaaggagac gagatgtggt
acaggggtgt tcgtctataa cgacgttgaa gcctggaggg acaggtacaa gtaccatcct
gactcccccc gtagattggc agcagcagtc aagcaagcct gggaagatgg tatctgcggg
atctcctctg tttcaagaat ggaaaacatc atgtggagat cagtagaagg ggagctcaac
gcaatcctgg aagagaatgg agttcaactg acggtcgttg tgggatctgt aaaaaacccc
atgtggagag gtccacagag attgcccgtg cctgtgaacg agctgcccca cggctggaag
gcttggggga aatcgtactt cgtcagagca gcaaagacaa ataacagctt tgtcgtggat
ggtgacacac tgaaggaatg cccactcaaa catagagcat ggaacagctt tcttgtggag
gatcatgggt tcggggtatt tcacactagt gtctggctca aggttagaga agattattca
ttagagtgtg atccagccgt tattggaaca gctgttaagg gaaaggaggc tgtacacagt
gatctaggct actggattga gagtgagaag aatgacacat ggaggctgaa gagggcccat
ctgatcgaga tgaaaacatg tgaatggcca aagtcccaca cattgtggac agatggaata
gaagagagtg atctgatcat acccaagtct ttagctgggc cactcagcca tcacaatacc
agagagggct acaggaccca aatgaaaggg ccatggcaca gtgaagagct tgaaattcgg
tttgaggaat gcccaggcac taaggtccac gtggaggaaa catgtggaac aagaggacca
tctctgagat caaccactgc aagcggaagg gtgatcgagg aatggtgctg cagggagtgc
acaatgcccc cactgtcgtt ctgggctaaa gatggctgtt ggtatggaat ggagataagg
cccaggaaag aaccagaaag caacttagta aggtcaatgg tgactgcagg atcaactgat
cacatggatc acttctccct t
KU955593 (full-length)
(SEQ ID NO: 7)
MKNPKKKSGGFRIVNMLKRGVARVSPFGGLKRLPAGLLLGHGPI
RMVLAILAFLRFTAIKPSLGLINRWGSVGKKEAMEIIKKFKKDLALMLRIINARKEKK
RRGTECSVGIVGLLLTTAMAVEVTRRGNAYYMYLDRSDAGEAISFPTTMGMNKCYIQI
MDLGHMCDATMSYECPMLDEGVEPDDVDCWCNTTSTWVVYGTCHHKKGEARRSBRAVT
LPSHSTRKLQTRSQTWLESREYTKHLIRVENWIFRNPGFALAAAAIAWLLGSSTSQKV
ITLVMILLIAPAYSIRCIGVSNRDFVEGMSGGTWVDVVLEHGGCVTVMAQDKPTVDIE
LVTTTVSNMAEVRSYCYEASISDMASDSRCPTQGEAYLDKQSDTQYVCKRTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLDCEPRTGLDFSDLYYLTMNNKHWLVHKEWF
HDIPLPWHAGADTGTPHWNNKEALVEFKDLHAKRQTVVVLGSQEGLVHTALAGLLEAE
MDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVG
EKKITHHWHRSGSTIGKAFEATVRGAKPMAVLGDTAWDFGSVGGALNSLGKGIHQIFG
AAFKSLFGGMSWFSQILIGTLLVWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSVKNPMWRGPQRLPVPVNELPHGWKAWGK
SYFVRAAKTNNSFVVDGDTLKECPLKHRAWNSFLVEDHGFGVFHTSVWLKVREDYSLE
CDPAVIGTAAKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFRAKDGCWYGMEIRPRKEPESNLVRSMVTA
GSTDHMDHFSLGVLVILLMVQEGLKKRMTTKIIISTSMAVLVAMILGGFSMSDLAKLA
ILMGATFAEMNTGGDVAHLALIAAFKVRPALLVSFIFRANWTPRESMLLALASCLLQT
AISALEGDLMVPINGFALAWLAIPAMVVPRTDNITLAILAALTPLARGTLLVAWRAGL
ATCGGFMLLSLKGKGSVKKNLPFVMALGLTAVRLVDPINVVGLLLLTRSGKRSWPPSE
VLTAVGLICALAGGFAKADIEMAGPMAAVGLLIVSYVVSGKSVDMYIERAGDITWEKD
AEVTGNSPRLDVALDESGDFSLVEDDGPPMREIILKVVLMAICGMNPIAIPFAAGAWY
VYVKTGKRSGALWDVPAPKEVKKGETTDGVYRVMTRRLLGSTQVGVGVMQEGVFHTMW
HVTKGSALRSGEGRLDPYWGDVKQDLVSYCGPWKLDAAWDGHSEVQLLAVPPGERARN
IQTLPGIFKTKDGDIGAVALDYPAGTSGSPILDKGGRVIGLYGNGVVIKNGSYVSAIT
QGRREEETPVECFEPSMLKKKQLTVLDLHPGAGKTRRVLPEIVREAIKTRLRTVILAP
TRVVAAEMEEALRGLPVRYMTTAVNVTHSGTEIVDLMCHATFTSRLLQPIRVPNYNLY
IMDEAHETDPSSIAARGYISTRVEMGEAAAIFMTATPPGTRDAFPDSNSPIMDTEVEV
PERAWSSGFDWVTDHSGKTVWFVPSVRNGNEIAACLTKAGKRVIQLSRKTFETEFQKT
KHQEWDFVVTTDISEMGANFKADRVIDSRRCLKPVILDGERVILAGPMPVTHASAAQR
RGRIGRNPNKPGDEYLYGGGCAETDEDHAHWLEARMLLDNIYLQDGLIASLYRPEADK
VAAIEGEFKLRTEQRKTFVELMKRGDLPVWLAYQVASAGITYTDRRWCFDGTTNNTIM
EDSVPAEVWTRYGEKRVLKPRWMDARVCSDHALLKSFKEFAAGKRGAAFGVMEALGTL
PGHMTERFQEAIDNLAVLMRAETGSRPYKAAAAQLPETLETIMLLGLLGTVSLGIFFV
LMRNKGIGKMGFGMVTLGASAWLMWLSEIEPARIACVLIVVFLLLVVLIPEPEKQRSP
QDNQMAIIIMVAVGLLGLITANELGWLERTKSDLSHLMGRREEGATIGFSMDIDLRPA
SAWAIYAALTTFITPAVQHAVTTSYNNYSLMAMATQAGVLFGMGKGMPFYAWDFGVPL
LMIGCYSQLTPLTLIVAIILLVAHYKYLIPGLQAAAARAAQKRTAAGIMKNPVVDGIV
VTDIDTMTIDPQVEKKMGQVLLIAVAYSSAILSRTAWGWGEAGALITAATSTLWEGSP
NKYWNSSTATSLCNIFRGSYLAGASLIYTVTRNAGLVKRRGGGTGETLGEKWKARLNQ
MSALEFYSYKKSGITEVCREEARRALKDGVATGGHAVSRGSAKLRWLVERGYLQPYGK
VIDLGCGRGGWSYYAATIRKVQEVKGYTKGGPGHEEPMLVQSYGWNIVRLKSGVDVEH
MAAEPCDTLLCDIGESSSSPEVEEARTLRVLSMVGDWLEKRPGAFCIKVLCPYTSTMM
ETLERLQRRYGGGLVRVPLSRNSTHEMYWVSGAKSNTIKSVSTTSQLLLGRMDGPRRP
VKYEEDVNLGSGTRAVVSCAEAPNMKIIGNRIERIRSEHAETWFFDENHPYRTWAYHG
SYEAPTQGSASSLINGVVRLLSKPWDVVTGVTGIAMTDTTPYGQORVEKEKVDTRVPD
PQEGTRQVMSMVSSWLWKELGKHKRPRVCTKEEFINKVRSNAALGAIFEEEKEWKTAV
EAVNDPRFWALVDKEREHHLRGECQSCVYNMMGKREKKQGEFGKAKGSRAIWYMWLGA
RFLEFEALGELNEDHWMGRENSGGGVEGLGLQRLGYVLEEMSRIPGGRMYADDTAGWD
TRISRFDLENEALITNQMENGHRALALAIIKYTYQNKVVKVLRPAEKGKTVMDIISRQ
DQRGSGQVVTYALNTFTNLVVQLIRNMEAEEVLEMQDLWLLRRSEKVTNWLQSNGWDR
LKRMAVSGDDCVVKPIDDRFAHALRFLNDMGKVRKDTQEWKPSTGWDNWEEVPFCSHH
FNKLHLKDGRSIVVPCRHQDELIGRARVSPGAGWSIRETACLAKSYAQMWQLLYFHRR
DLRLMANAICSSVPVDWVPTGRTTWSIHGKGEWMTTEDMLVVNNRVWIEENDHMEDKT
PVTKWTDIPYLGKREDLWCGSLIGHRPRTTWAENIKNTVNMMRRIIGDEEKYVDYLST
QVRYLGEEGSTPGVL
(SEQ ID NO: 2)
agttgttgat ctgtgtgaat cagactgcga cagttcgagt ttgaagcgaa agctagaaac
agtatcaaca ggttttattt tggatttgga aacgagagtt tctggtcatg aaaaacccaa
agaagaaatc cggaggattc cggattgtca atatgctaaa acgcggagta gcccgtgtga
gcccctttgg gggcttgaag aggctgccag ccggacttct gctgggtcat gggcccatca
ggatggtctt ggcgattcta gcctttttga gattcacggc aatcaagcca tcactgggtc
tcatcaatag atggggttca gtggggaaaa aagaggctat ggaaataata aagaagttta
agaaagatct ggctgccatg ctgagaataa tcaatgctag gaaggagaag aagagacgag
gcacagatac tagtgtcgga attgttggcc tcctgctgac cacagccatg gcagtggagg
tcactagacg tgggaatgca tactatatgt acttggacag aagcgatgct ggggaggcca
tatcttttcc aaccacaatg gggatgaata agtgttatat acagatcatg gatcttggac
acatgtgtga tgccaccatg agctatgaat gccctatgct ggatgagggg gtagaaccag
atgacgtcga ttgttggtgc aacacgacgt caacttgggt tgtgtacgga acctgccacc
acaaaaaagg tgaagcacgg agatctagaa gagctgtgac gctcccctcc cattccacta
ggaagctgca aacgcggtcg cagacctggt tggaatcaag agaatacaca aagcacctga
ttagagtcga aaattggata ttcaggaacc ctggcttcgc gttagcagca gctgccatcg
cttggctttt gggaagctca acgagccaaa aagtcatata cttggtcatg atactgctga
ttgccccggc atacagcatc aggtgcatag gagtcagcaa tagggacttt gtggaaggta
tgtcaggtgg gacttgggtt gatgttgtct tggaacatgg aggttgtgtt accgtaatgg
cacaggacaa accgactgtc gacatagagc tggttacaac aacagtcagc aacatggegg
aggtaagatc ctactgctat gaggcatcaa tatcggacat ggcttcggac agccgctgcc
caacacaagg tgaagcctac cttgacaagc aatcagacac tcaatatgtc tgcaaaagaa
cgttagtgga cagaggctgg ggaaatggat gtggactttt tggcaaaggg agcctggtga
catgcgctaa gtttgcttgc tctaagaaaa tgaccgggaa gagcatccag ccagagaatc
tggagtaccg gataatgctg tcagttcatg gctcccagca cagtgggatg atcgttaatg
atacaggaca tgaaactgat gagaatagag cgaaggttga gataacgccc aattcaccaa
gagccgaagc caccctgggg ggttttggaa gcctaggact tgattgtgaa ccgaggacag
gccttgactt ttcagatttg tattacttga ctatgaataa caagcactgg ttggttcaca
aggagtggtt ccacgacatt ccattacctt ggcatgctgg ggcagacacc ggaactccac
actggaacaa caaagaagca ctggtagagt tcaaggacgc acatgccaaa aggcagactg
tcgtggttct agggagtcaa gaaggagcag ttcacacggc ccttgctgga gctctggagg
ctgagatgga tggtgcaaag ggaaggctgt cctctggcca cttgaaatgt cgcctgaaaa
tggataaact tagattgaag ggcgtgtcat actccttgtg taccgcagog ttcacattca
ctaagatccc ggctgaaaca ctgcacggga cagtcacagt ggaggtacag tacgcaggga
cagatggacc ttgcaaggtt ccagctcaga tggcggtgga catgcaaact ctgaccccag
ttgggaggtt gataaccgct aaccctgtaa tcactgaaag cactgagaac tccaagatga
tgctggaact ggatccacca tttggggact cttacattgt cataggagtc ggggaaaaga
agatcaccca ccactggcac aggagtggca gcaccattgg aaaagcattt gaagccactg
tgagaggtgc caagagaatg gcagtcttgg gagacacagc ctgggacttt ggatcagttg
ggggtgctct caactcactg ggcaagggca tccatcaaat ttttggagca gctttcaaat
cattgtttgg aggaatgtcc tggttctcac aaattctcat tggaacgttg ctggtgtggt
tgggtctgaa tacaaagaat ggatctattt cccttatgtg cttggcctta gggggagtgt
tgatcttctt atccacagcc gtctctgctg atgtggggtg ctoggtggac ttctcaaaga
aggaaacgag atgcggtaca ggggtgttcg tctataacga cgttgaagct tggagggaca
ggtacaagta ccatcctgac tcccctcgta gattggcagc agcagtcaag caagcctggg
aagatgggat ctgtgggatc tcctctgttt caagaatgga aaacatcatg tggagatcag
tagaagggga gctcaacgca atcctggaag agaatcgagt tcaactgacg gtcgttgtgg
gatctgtaaa aaaccccatg tggagaggtc cacagagatt gcccgtgcct gtgaacgagc
tgccccatgg ctggaaggct tgggggaaat cgtacttcgt cagggcagca aagacaaata
acagctttgt cgtggatggt gacacactga aggaatgccc actcaaacat agagcatgga
acagctttct tgtggaggat catgggttcg gggtatttca cactagtgtc tggctcaagg
ttagagaaga ttattcatta gagtgtgatc cagccgtcat tggaacagcc gctaagggaa
aggaggctgt gcacagtgat ctaggctact ggattgagag tgagaagaac gacacatgga
ggctgaagag ggcccacctg atcgagatga aaacatgtga atggccaaag tcccacacat
tgtggacaga tggaatagaa gaaagtgatc tgatcatacc caagtcttta gctgggccac
tcagccatca caacaccaga gagggctaca ggacccaaat gaaagggcca tggcatagtg
aagagcttga aattcggttt gaggaatgcc caggcactaa ggtccacgtg gaggaaacat
gtggaacaag aggaccatct ctgagatcaa ccactgcaag cggaagggtg atcgaggaat
ggtgctgcag ggagtgcaca atgcccccac tgtcgttccg ggctaaagat ggttgttggt
atggaatgga gataaggccc aggaaagaac cagaaagtaa cttagtaagg tcaatggtga
ctgcaggatc aactgatcac atggatcact tctcccttgg agtgcttgtg attctgctca
tggtacagga agggctaaag aagagaatga ccacaaagat catcataagc acatcaatgg
cagtgctggt agctatgatc ctgggaggat tttcaatgag tgacctggct aagcttgcaa
ttttgatggg tgccaccttc gcggaaatga acactggagg agatgttgct catctggcgc
tgatagcggc attcaaagtc agacctgcgt tgctggtatc tttcattttc agagctaatt
ggacaccccg tgagagcatg ctgctggcct tggcctcgtg tcttctgcaa actgcgatct
ccgccttgga aggcgacctg atggttccca tcaatggttt tgctttggcc tggttggcaa
tacgagcgat ggttgttcca cgcactgaca acatcacctt ggcaatcctg gctgctctga
caccactggc ccggggcaca ctgcttgtgg cgtggagagc aggccttgct acttgcgggg
ggttcatgct cctttctctg aaggggaaag gcagtgtgaa gaagaactta ccatttgtca
tggccctggg actaaccgct gtgaggctgg tcgaccccat caacgtggtg ggactgctgt
tgctcacaag gagtgggaag cggagctggc cccctagtga agtactcaca gctgttggcc
tgatatgcgc attggctgga gggttcgcca aggcggatat agagatggct gggcccatgg
ccgcggtcgg tctgctaatt gtcagttacg tggtctcagg aaagagtgtg gacatgtaca
ttgaaagagc aggtgacatc acatgggaaa aagatgcgga agtcactgga aacagtcccc
ggctcgatgt ggcactagat gagagtggtg atttctccct agtggaggat gatggtcccc
ccatgagaga gatcatactc aaagtggtcc tgatggccat ctgtggcatg aacccaatag
ccataccctt tgcagctgga gcgtggtacg tgtatgtgaa gactggaaaa aggagtggtg
ctctatggga tgtgcctgct cccaaggaag taaaaaaggg ggagaccaca gatggagtgt
acagagtaat gactcgtaga ctgctaggtt caacacaagt tggagtggga gtcatgcaag
agggggtctt ccacactatg tggcacgtca caaaaggatc cgcgctgaga agcggtgaag
ggagacttga tccatactgg ggagatgtca agcaggatct ggtgtcatac tgtggtccat
ggaagctaga tgccgcctgg gacgggcaca gcgaggtgca gctcttggcc gtgccccccg
gagagagagc gaggaacatc cagactctgc ccggaatatt taagacaaag gatggggaca
ttggagcagt tgcgctggac tacccagcag gaacttcagg atctccaatc ctagataagt
gtgggagagt gataggactc tatggtaatg gggtcgtgat caaaaatggg agttacgtta
gtgccatcac ccaagggagg agggaggaag agactcctgt tgagtgcttc gagccttcga
tgctgaagaa gaagcagcta actgtcttag acttgcatcc tggagctggg aaaaccagga
gagttcttcc tgaaatagtc cgtgaagcca taaaaacaag actccgcact gtgatcttag
ctccaaccag ggttgtcgct gctgaaatgg aggaagccct tagagggctt ccagtgcgtt
atatgacaac agcagtcaat gtcacccatt ctgggacaga aatcgttgac ttaatgtgcc
atgccacctt cacttcacgt ctactacagc caatcagagt ccccaactat aatctgtata
ttatggatga ggcccacttc acagatccct caagtatagc agcaagagga tacatttcaa
caagggttga gatgggcgag gcggctgcca tcttcatgac tgccacgcca ccaggaaccc
gtgacgcatt cccggactcc aactcaccaa ttatggacac cgaagtggaa gtcccagaga
gagcctggag ctcaggcttt gattgggtga cggatcattc tggaaaaaca gtttggtttg
ttccaagcgt gaggaatggc aatgagatcg cagcttgtct gacaaaggct ggaaaacggg
tcatacagct cagcagaaag acttttgaga cagagttcca gaaaacaaaa catcaagagt
gggacttcgt cgtgacaact gacatttcag agatgggcgc caactttaaa gctgaccgtg
tcatagattc caggagatgc ctaaagccgg tcatacttga tggcgagaga gtcattctgg
ctggacccat gcctgtcaca catgccagcg ctgcccagag gagggggogc ataggcagga
accccaacaa acctggagat gagtatctgt atggaggtgg gtgcgcagag actgatgaag
accatgcaca ctggcttgaa gcaagaatgc ttcttgacaa catttacctc caagatggcc
tcatagcctc gctctatcga cctgaggccg acaaagtagc agctattgag ggagagttca
agcttaggac ggagcaaagg aagacctttg tggaactcat gaaaagagga gatcttcctg
tttggctggc ctatcaggtt gcatctgccg gaataaccta cacagataga agatggtgct
ttgatggcac gaccaacaac accataatgg aagacagtgt gccggcagag gtgtggacca
gatacggaga gaaaagagtg ctcaaaccga ggtggatgga cgccagagtt tgttcagatc
atgcggccct gaagtcattc aaagagtttg ccgctgggaa aagaggagcg gcctttggag
tgatggaagc cctgggaaca ctgccaggac atatgacaga gagattccag gaggccattg
acaacctcgc tgtgctcatg cgggcagaga ctggaagcag gccctacaaa gccgcggcgg
cccaattacc ggagacccta gagactatca tgcttttggg gttgctggga acagtctcgc
tgggaatctt tttcgtcttg atgcggaaca agggcatagg gaagatgggc tttggaatgg
tgactcttgg ggccagcgca tggcttatgt ggctctcgga aattgagcca gccagaattg
catgtgtcct cattgttgtg ttcctattgc tggtggtgct catacctgag ccagaaaagc
aaagatctcc ccaggacaac caaatggcaa tcatcatcat ggtagcagtg ggtcttctgg
gcttgattac cgccaatgaa ctcggatggt tggagagaac aaagagtgac ctaagccatc
taatgggaag gagagaggag ggggcaacta taggattctc aatggacatt gacctgcggc
cagcctcagc ttgggctatc tatgctgctc tgacaacttt cattacccca gccgtccaac
atgcagtgac cacttcatac aacaactact ccttaatggc gatggccacg caagctggag
tgttgttcgg tatgggtaaa gggatgccat tctatgcatg ggactttgga gtcccgctgc
taatgatagg ttgctactca caattaacac ccctgaccct aatagtggcc atcattttgc
tcgtggcgca ctacatgtac ttgatcccag ggctgcaggc agcagctgcg cgtgctgccc
agaagagaac ggcagctggc atcatgaaga accctgttgt ggatggaata gtggtgactg
acattgacac aatgacaatt gacccccaag tggagaaaaa gatgggacag gtgctactca
tagcagtagc tgtctccagc gccatactgt cgcggaccgc ctgggggtgg ggtgaggctg
gggccctgat cacagctgca acttccactt tgtgggaggg ctctccgaac aagtactgga
actcctccac agccacctca ctgtgtaaca tttttagggg aagctacttg gctggagctt
ctctaatcta cacagtaaca agaaacgctg gcttggtcaa gagacgtggg ggtggaacgg
gagagaccct gggagagaaa tggaaggccc gcctgaacca gatgtcggcc ctggagttct
actcctacaa aaagtcaggc atcaccgagg tgtgcagaga agaggcccgc cgcgccctca
aggacggtgt ggcaacggga ggccacgctg tgtcccgagg aagtgcaaag ctgagatggt
tggtggagag gggatacctg cagccctatg gaaaggtcat tgatcttgga tgtggcagag
ggggctggag ttactatgcc gccaccatcc gcaaagttca agaagtgaaa ggatacacaa
aaggaggccc tggtcatgaa gaacccatgt tggtgcaaag ctatgggtgg aacatagtcc
gtcttaagag tggggtggac gtctttcata tggcggctga gccgtgtgac acgttgctgt
gtgatatagg tgagtcatca tctagtcctg aagtggaaga agcacggacg ctcagagtcc
tctccatggt gggggattgg cttgaaaaaa gaccaggagc cttttgtata aaagtgttgt
gcccatacac cagcactatg atggaaaccc tggagcgact gcagcgtagg tatgggggag
gactggtcag agtgccactc tcccgcaact ctacacatga gatgtactgg gtctctggag
cgaaaagcaa caccataaaa agtgtgtcca ccacgagcca gctccttttg gggcgcatgg
acgggcccag gaggccagtg aaatatgaag aggatgtgaa tctcggctct ggcacgcggg
ctgtggtaag ctgcgctgaa gctcccaaca tgaagatcat tggtaaccgc attgagagga
tccgcagtga gcacgcggaa acgtggttct ttgacgagaa ccacccatat aggacatggg
cttaccatgg aagctacgag gcccccacac aagggtcagc gtcctctcta ataaacgggg
ttgtcaggct cctgtcaaaa ccctgggatg tggtgactgg agtcacagga atagccatga
ccgacaccac accgtatggt cagcaaagag ttttcaagga aaaagtggac actagggtgc
cagaccccca agaaggcact cgtcaggtta tgagcatggt ctcttcctgg ttgtggaaag
agttaggcaa acacaaacgg ccacgagtct gtaccaaaga agagttcatc aacaaggttc
gtagcaacgc agcattaggg gcaatatttg aagaggaaaa agagtggaag actgcagtgg
aagctgtgaa cgatccaagg ttctgggctc tagtggacaa ggaaagagag caccacctga
gaggagagtg ccagagctgt gtgtacaaca tgatgggaaa aagagaaaag aaacaagggg
aatttggaaa ggccaagggc agccgcgcca tctggtacat gtggctaggg gctagatttc
tagagttcga agcccttgga ttcttgaacg aggatcactg gatggggaga gagaattcag
gaggtggtgt tgaagggcta ggattacaaa gactcggata tgtcttagaa gagatgagtc
gcataccagg aggaaggatg tatgcagatg atactgctgg ctgggacacc cgcatcagca
ggtttgatct ggagaatgaa gctctaatca ccaaccaaat ggagaaaggg cacagggcct
tggcattggc cataatcaag tacacatacc aaaacaaagt ggtaaaggtc cttagaccag
ctgaaaaagg gaagacagtt atggacatta tttcaagaca agaccaaagg gggagcggac
aagttgtcac ttacgctctt aatacattta ccaacctagt ggtgcagctc attcggaata
tggaggctga ggaagttcta gagatgcaag acttgtggct gctgcggagg tcagagaaag
tgaccaactg gttgcagagc aatggatggg ataggctcaa acgaatggca gtcagtggag
atgattgcgt tgtgaaacca attgatgata ggtttgcaca tgctctcagg ttcttgaatg
atatgggaaa agttaggaag gacacacaag agtggaagcc ctcaactgga tgggacaact
gggaagaagt tccgttttgc tcccaccact tcaacaagct ccatctcaag gacgggaggt
ccattgtggt tocctgccgc caccaagatg aactgattgg ccgagctcgc gtctcaccgg
gggcgggatg gagcatccgg gagactgctt gcctagcaaa atcatatgcg caaatgtggc
agctccttta tttccacaga agggacctcc gactgatggc caatgccatt tgttcatctg
tgccagttga ctgggttcca actgggagaa ctacctggtc aatccatgga aagggagaat
ggatgaccac tgaagacatg cttgtggtgt ggaacagagt gtggcttgag gagaacgacc
acatggaaga caagacccca gttacgaaat ggacagacat tccctatttg ggaaaaaggg
aagacttgtg gtgtgggtct ctcatagggc acagaccgcg caccacctgg gctgagaaca
ttaaaaacac agtcaacatg atgcgtagga tcataggtga tgaagaaaag tacgtggact
acctatccac ccaagttcgc tacttgggcg aagaagggtc cacacctgga gtgctataag
caccaatctt agtgttgtca ggcctgctag tcagccacag cttggggaaa gctgtgcagc
ctgtgacccc cccaggagaa gctgggaaac caagcccata gtcaggccga gaacgccatg
gcacggaaga agccatgctg cctgtgagcc cctcagagga cactgagtca aaaaacccca
cgcgcttgga ggcgcaggat gggaaaagaa ggtggcgacc ttccccaccc tttaatctgg
ggcctgaact ggagatcagc tgtggatctc cagaagaggg actagtggtt agaggagacc
ccccggaaaa cgcaaaacag catattgacg ctgggaaaga ccagagactc catgagtttc
caccacgctg gccgccaggc acagatcgcc gaatagcggc ggccggtgtg gggaaatcca
tgggtct
KU866423
(SEQ ID NO: 8)
MYNPKKKSGGFRIVNMLFRGVARVSPFGGLKRLPAGLLLGHGPI
RMVLAILAFLRFTAINTSLGLINRWGSVGKKEAMEIIKKFKKDLAAMLRIINARKEKK
RRGADTNVGIVGLLLTTAMAAEVTRRGSAYYMYLDRNDAGEAISFPTTLGMNKCYIQI
MDLGHMCDATMSYECPMLDEGVEPDDVDCWCNTTSTWVVYGTCHHKKGEARRSRRAVT
LPSHSTRKLQTRSQTWLESREYTKHLIRVENWIFRNPGFALAAAAIAWLLGSSTSQKV
IYLVMILLIAPAYSIRCIGVSNRDFVEGMSGGTWYDWILEHGGCVTVMAQDKPTVDIE
LVTTTVSNMAEVRSYCYEASISDMASDSRCPTQGEAYLDKQSDTQYVCERTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLDCEPRTGLDFSDLYYLTMNNKHWLVHKEWF
HDIPLPWHAGADTGTPHWNNKEALVEEKDAHAKRQTVVVLGSQEGAVHTALAGALEAE
MDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVG
EKKITHHWHRSGSTIGKAFEATVRGARRMAVLGDTAWDFGSVGGALNSLGKGIHQIFG
AAFKSLFGGMSWFSQILIGTLLMWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSYKNPMWRGPQRLPVPVNELPHGWKAWGK
SYFVRAAKTNNSFVVDGDTLKECPLKHRAWNSFLVEDHGFGVEHTSVWLKVREDYSLE
CDPAVIGTAVKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFQAKDGCWYGMEIRPRKEPESNLVRSMVTA
GSTDHKDHFSLGVLVILLMVQEGLKKRMTTKIIISTSMAVLVAMILGGFSMSDLAKLA
ILMGATFAEMNTGGDVAHLALIAAFKYRPALINSFIFRANWTPRESMLLALASCLLQT
AISALEGDLMVLINGFALAWLAIRAMVVPRTDNITLAILAALTPLARGTLLVAWRAGL
ATCGGFMLLSLKGKGSVKKNLPFVMALGLTAVRLVDPINVVGLLLLTRSGKRSWPPSE
VLTAVGLICALAGGFAKADIEMAGPMAAVGLLIVSYVVSGKSVDMYIERAGDITWEKD
AEVTGNSPRLDVALDESGDFSLVEDDGPPMREIILKVVLMTICGMNPIAIPEAAGAWY
VYVKTGKRSGALWDVPAPKEVKKGETTDGVYRVMTRRLLGSTQVGVGVMQEGVFHTMW
HVTKGSALRSGEGRLDPYWGDVKQDLVSYCGPWKLDAAWDGHSEVQLLAVPPGERARN
IQTLPGIFKTKDGDIGAVALDYPAGTSGSPILDKCGRVIGLYGNGVVIKNGSTVSAIT
QGRREEETPVECFEPSMLKKKQLTVLDLHPGAGKTRRVLPEIVREAIKTRLRTVILAP
TRVVAAEMEEALRGLPVRYMTTAVNVTHSGTEIVDLMCHATFTSRLLQPIRVPNYNLY
IMDEAHFTDPSSIAARGYISTRVEMGEAAAIFMTATPPGTRDAFPDSNSPIMDTEVEV
PERAWSSGEDWVTDHSGKTVWFVPSVRNGNEIAACLTKAGKRVIQLSRKTFETEFQKT
KHQEWDEVVTTDISEMGANFKADRVIDSRRCLKPVILDGERVILAGPMPVTHASAAQR
RGRIGRNPNKPGDEYLYGGGCAETDEDHAHWLEARMLLDNIYLQDGLIASLYRPEADK
VAAIEGEFKLRTEQRKTFVELMKRGDLPVWLAYQVASAGITYTDRRWCFDGTTNNTIM
EDSVPAEVWTRHGEKRVLKPRWMDARVCSDHAALKSFKEFAAGKRGAAFGVMEALGTL
PGHMTERFQEAIDNLAVLMRAETGSRPYKAAAAQLPETLETIMLLGLLGTVSLGIFFV
LMRNKGIGKMGFGMVTLGASAWLMWLSEIEPARIACVLIVVFLLLVVLIPEPEKQRSP
QDNQMAIIIMVAVGLLGLITANELGWLERTKSDLSHLMGRREEGATIGESMDIDLRPA
SAWAIYAALTTFITPAVQHAVTTSYNNYSLMAMATQAGVLFGMGKGMPFYAWDFGVPL
LMIGCYSQLTPLTLIVAIILLVAHYMYLIPGLQAAAARAAQKRTAAGIMKNPVVDGIV
VTDIDTMTIDPQVEKKMGQVLLIAVAVSSAILSRTAWGWGEAGALITAATSTLWEGSP
NKYWNSSTATSLCNIFRGSYLAGASLIYTV7RNAGINKRRGGGTGETLGEKWKARLNQ
MSALEYYSYKKSGITEVCREEARRALKDGVATGGHAVSRGSAKLRWLVERGYLQPYGK
VIDLGCGRGGWSTYAATIRKVOEVKGYTKGGPGEEEPMLVQSYGWNIVRIKSGVDVFH
KLAEPCDTLLCDIGESSSSPEVEEARTLRVLSMVGDWIEKRPGAFCIKVLCPYTSTMM
ETLERIQRRYGGGIVRVPLSRNSTHEMYWVSGAKSNTIKSVSTTSQLLLGRMDGPRRP
VKYEEDVNLGSGTRAVVSCAEAPNKKIIGNRIERIRSEHAETWFFDENHPYRTWAYHG
SYEAPTQGSASSLINGVVRLLSKPWDVVTGVTGIAMTDTTPYGQQRVFKEKVDTRVPD
PQEGTRQVMSMVSSWLWKELGKHKRPRVCTKEEFINKVRSNAALGAIFEEEKEWKTAV
EAVNDPRFWALVDNEREHHLRGECQSCVYNMMGKREKKGQEFGKAKGSRAIWYMWLGA
RFLEFEALGFLNEDHWMGRENSGGGVEGLGLQRLGYVLEEMSRIPGGRMYADDTAGWD
TRISRFDLENEALITNQMEKGHRAIALAIIKYTYQNKVVKVLRPAEKGKTVMDIISRQ
DQRGSGQVVTYALNTFTNLVVQLIRSMEAEEVLEMQDLWLLRRSEKVTNWLQSNGWDR
LKRMAVSGDDCVVRPIDDRFAHALRFLNDMGKVRKDTQEWKPSTGNDNWEEVPFCSHH
FNKLHLKDGRSIVVPCRHQDELIGRARVSPGAGWSIRETACLAKSYAQMNQLLYFHRR
DLRLMANAICSSVPVDWVPTGRTTWSIHGKGEWMTTEDMINVWNRVWIEENDHMEDKT
PVTKWTDIPYLGKREDLWCGSLIGHRPRTTWAENIKNTVNMVRRIIGDEEKYMDYLST
WRYLGEEGSTPGVL
(SEQ ID NO: 3)
atgaaaaacc oaaaaaagaa atccggagga ttccggattg tcaatatgct aaaacgcgga
gtagcccgtg tgagcccctt tgggggcttg aagaggctgc cagccggact tctgctgggt
catgggccca tcaggatggt cttggcgatt ctagccttct tgagattcac ggcaatcaag
ccatcactgg gtctcatcaa tagatggggt tcagtgggga aaaaagaggc tatggaaata
ataaagaagt tcaagaaaga tctggctgcc atgctgagaa taatcaatgc taggaaggag
aagaagagac gaggcgcaga tactaatgtc ggaattgttg gcctcctgct gaccacagct
atggcagcgg aggtcactag acgtgggagt gcatactata tgtacttgga cagaaacgat
gctggggagg ccatatcttt tccaaccaca ttggggatga ataagtgtta tatacagatc
atggatcttg gacacatgtg tgatgccacc atgagctatg aatgccctat gctggatgag
ggggtggaac cagatgacgt cgattgttgg tgcaacacga cgtcaacttg ggttgtgtac
ggaacctgcc atcacaaaaa aggtgaagca cggagatcta gaagagctgt gacgctcccc
toccattcca ctaggaagct gcaaacgcgg tcgcaaactt ggttggaatc aagagaatac
acaaagcact tgattagagt cgaaaattgg atattcagga accctggctt cgcgttagca
gcagctgcca tcgcttggct tttgggaagc tcaacgagcc aaaaagtcat atacttggtc
atgatactgc tgattgcccc ggcatacagc atcaggtgca taggagtcag caatagggac
tttgtggaag gtatgtcagg tgggacttgg gttgatgttg tcttggaaca tggaggttgt
gtcaccgtaa tggcacagga caaaccgact gtcgacatag agctggttac aacaacagtc
agcaacatgg cggaggtaag atcctactgc tatgaggcat caatatcgga catggcttcg
gacagccgct gcccaacaca aggtgaagcc taccttgaca agcaatcaga cactcaatat
gtctgcaaaa gaacgttagt ggacagaggc tggggaaatg gatgtggact ttttggcaaa
gggagcctgg tgacatgcgc taagtttgca tgctccaaga aaatgaccgg gaagagcatc
cagccagaga atctggagta ccggataatg ctgtcagttc atggctccca gcacagtggg
atgatcgtta atgacacagg acatgaaact gatgagaata gagcgaaggt tgagataacg
cccaattcac caagagccga agccdccctg gggggttttg gaagcctagg acttgattgt
gaaccgagga caggccttga cttttcagat ttgtattact tgactatgaa taacaagcac
tggttggttc acaaggagtg gttccacgac attccattac cttggcacgc tggggcagac
accggaactc cacactggaa caacaaagaa gcactggtag agttcaagga cgcacatgcc
aaaaggcaaa ctgtcgtggt tctagggagt caagaaggag cagttcacac ggcccttgct
ggagctctgg aggctgagat ggatggtgca aagggaaggc tgtcctctgg ccacttgaaa
tgtcgcctga aaatggataa acttagattg aagggcgtgt catactcctt gtgtaccgca
gcgttcacat tcaccaagat cccggctgaa acactgcacg ggacagtcac agtggaggta
cagtacgcag ggacagatgg accttgcaag gttccagctc agatggcggt ggacatgcaa
actctgaccc cagttgggag gctgataacc gctaaccccg taatcactga aagcactgag
aactccaaga tgatgctgga acttgatcca ccatttgggg actcttacat tgtcatagga
gtcggggaga agaagatcac ccaccactgg cacaggagtg gcagcaccat tggaaaagca
tttgaagcca ctgtgagagg tgccaggaga atggcagtct tgggagacac agcctgggac
tttggatcag ttggaggcgc tctcaactca ttgggcaagg gcatccatca aatttttgga
gcagctttca aatcattgtt tggaggaatg tcctggttct cacaaattct cattggaacg
ttgctgatgt ggttgggtct gaacacaaag aatggatcta tttcccttat gtgcttggcc
ttagggggag tgttgatctt cttatccaca gccgtctctg ctgatgtggg gtgctcggtg
gacttctcaa agaaggagac gagatgcggt acaggggtgt tcgtctataa cgacgttgaa
gcctggaggg acaggtacaa gtaccatcct gactcccocc gtagattggc agcagcagtc
aagcaagcct gggaagatgg tatctgtggg atctcctctg tttcaagaat ggaaaacatc
atgtggagat cagtagaagg ggagctcaac gcaatcctgg aagagaatgg agttcaactg
acggtcgttg tgggatctgt aaaaaacccc atgtggagag gtccacagag attgcccgtg
cctgtgaacg agctgcccca cggctggaag gcttggggga aatcgtactt cgtcagagca
gcaaagacaa ataacagctt tgtcgtggat ggtgacacac tgaaggaatg cccactcaaa
catagagcat ggaacagctt tcttgtggag gatcatgggt tcggggtatt toacactagt
gtctggctca aggttagaga agattattca ttagagtgtg atccagccgt tattggaaca
gctgttaagg gaaaggaggc tgtacacagt gatctaggct actggattga gagtgagaag
aatgacacat ggaggctgaa gagggcccat ctgatcgaga tgaaaacatg tgaatggcca
aagtcccaca cattgtggac agatggaata gaagagagtg atctgatcat acccaagtct
ttagctgggc cactcagcca tcacaatacc agagagggct acaggaccca aatgaaaggg
ccatggcaca gtgaagagct tgaaattcgg tttgaggaat gcccaggcac caaggtccac
gtggaggaaa catgtggaac aagaggacca tctctgagat caaccacagc aagcggaagg
gtgatcgagg aatggtgctg cagggagtgc acaatgcccc cactgtcgtt ccaggctaaa
gatggctgtt ggtatggaat ggagataagg cccaggaaag aaccagaaag taacttagta
aggtcaatgg tgactgcagg atcaactgat cacatggatc acttctccct tggagtgctt
gtgattctgc tcatggtgca ggaagggctg aagaagagaa tgaccacaaa gatcatcata
agcacatcaa tggcagtgct ggtagctatg atcctgggag gattttcaat gagtgacctg
gctaagcttg caattttgat gggtgccacc ttcgcggaaa tgaacactgg aggagatgta
gctcatctgg cgctgatagc ggcattcaaa gtcagaccag cgttgctggt atctttcatc
ttcagagcta attggacacc ccgtgaaagc atgctgctgg ccttggcctc gtgtcttttg
caaactgcga tctccgcctt ggaaggcgac ctgatggttc tcatcaatgg ttttgctttg
gcctggttgg caatacgagc gatggttgtt ccacgcactg ataacatcac cttggcaatc
ctggctgctc tgacaccact ggcccggggc acactgcttg tggcgtggag agcaggcctt
gctacttgcg gggggtttat gctcctctct ctgaagggaa aaggcagtgt gaagaagaac
ttaccatttg tcatggccct gggactaacc gctgtgaggc tggtcgaccc catcaacgtg
gtgggactgc tgttgctcac aaggagtggg aagcggagct ggccccctag cgaagtactc
acagctgttg gcctgatatg cgcattggct ggagggttcg ccaaggcaga tatagagatg
gctgggccca tggccgcggt cggtctgcta attgtcagtt acgtggtctc aggaaagagt
gtggacatgt acattgaaag agcaggtgac atcacatggg aaaaagatgc ggaagtcact
ggaaacagtc cccggcttgc tgtggcgcta gatgagagtg gtgatttctc cctggtggag
gatgacggtc cccccatgag agagatcata ctcaaggtgg tcctgatgac catctgtggc
atgaacccaa tagccatacc ctttgcagct ggagcgtggt acgtatacgt gaagactgga
aaaaggagtg gagctctatg ggatgtgcct gctcccaagg aagtaaaaaa gggggagacc
acagatggag tgtacagagt gatgactcgt agactgctag gttcaacaca agttggagtg
ggagttatgc aagagggggt ctttcacacc atgtggcacg tcacaaaagg atccgcgctg
agaagcggtg aagggagact tgatccatac tggggagatg tcaagcagga tctggtgtca
tactgtggtc catggaagct agatgccgcc tgggacgggc acagcgaggt gcagctcttg
gccgtgcccc ccggagagag agcgaggaac atccagactc tgcccggaat atttaagaca
aaggatgggg acattggagc ggttgcgctg gattacccag caggaacttc aggatctcca
atcctagaca agtgtgggag agtgatagga ctttatggca atggggtcgt gatcaaaaat
gggagttatg ttagtgccat cacccaaggg aggagggagg aagagactcc tgttgagtgc
ttcgagcctt cgatgctgaa gaagaagcag ctaactgtct tagacttgca tcctggagct
gggaaaacca ggagagttct tcctgaaata gtccgtgaag ccataaaaac aagactccgt
actgtgatct tagctccaac cagggttgtc gctgccgaaa tggaggaagc ccttagaggg
cttccagtgc gttatatgac aacagcagtc aatgtcaccc actctggaac agaaatcgtc
gacttaatgt gccatgccac cttcacttca cgtctactac agccaatcag agtccccaac
tataatctgt atattatgga tgaggcccac ttcacagatc cctcaagtat agcagcaaga
ggatacattt caacaagggt tgagatgggc gaggcggctg ccatcttcat gaccgccacg
ccaccaggaa cccgtgacgc atttccggac tccaactcac caattatgga caccgaagtg
gaagtcccag agagagcctg gagctcaggc tttgattggg tgacggatca ttctggaaaa
acagtctggt ttgttccaag cgtgaggaac ggcaatgaga tcgcagcttg tctgacaaag
gctggaaaac gggtcataca gctcagcaga aagacttttg agacagagtt ccagaaaaca
aaacatcaag agtgggactt tgtcgtgaca actgacattt cagagatggg cgccaacttt
aaagctgacc gtgtcataga ttccaggaga tgcctaaagc cggtcatact tgatggcgag
agagtcattc tggctggacc catgcctgtc acacatgcca gcgctgccca gaggaggggg
cgcataggca ggaatcccaa caaacctgga gatgagtatc tgtctggagg tgggtgcgca
gagactgacg aagaccatgc acactggctt gaagcaagaa tgctccttga caatatttac
ctccaagatg gcctcatagc ctcgctctat cgacctgagg ccgacaaagt agcagccatt
gagggagagt tcaagcttag gacggagcaa aggaagacct ttgtggaact catgaaaaga
ggagatcttc ctgtttggct ggcctatcag gttgcatctg ccggaataac ctacacagat
agaagatggt gctttgatgg cacgaccaac aacaccataa tggaagacag tgtgccggca
gaggtgtgga ccagacacgg agagaaaaga gtgctcaaac cgaggtggat ggacgccaga
gtttgttcag atcacgcggc cctgaagtca ttcaaggagt ttgccgctgg gaaaagagga
gcggcttttg gagtgatgga agccttggga acactgccag gacacatgac agagagattc
caggaagcca ttgacaacct cgctgtgctc atgcgggcag agactggaag caggccttac
aaagccgcgg cggcccaatt gccggagacc ctagagacca ttatgctttt ggggttgctg
ggaacagtct cgctgggaat ctttttcgtc ttgatgagga acaaccgcat accgaagatg
ggctttggaa tggtgactct tcccgccagc gcatggctca tgtggctctc ggaaattgag
ccagccagaa ttgcatgtgt cctcattgtt gtgttcctat tgctggtggt gctcatacct
gagccagaaa agcaaagatc tccccaggac aaccaaatgg caatcatcat catggtagca
gtaggtcttc tgggcttgat taccgccaat gaactcggat ggttggagag aacaaagagt
gacctaagcc atctaatggg aaggagagag gagggggcaa ccataggatt ctcaatggac
attgacctgc ggccagcctc agcttgggcc atctacgctg ccttgacaac tttcattacc
ccagccgtcc aacatgcagt gaccacttca tacaacaact actccttaat ggcgatggcc
acgcaagctg gagtgttgtt tggtatgggc aaagggatgc cattctacgc atgggacttt
ggagtcccgc tgctaatgat aggttgctac tcacaattaa cacccctgac cctaatagta
gccatcattt tgctcgtggc gcactacatg tacttgatcc cagggctgca ggcagcagct
gcgcgtgctg cccagaagag aacggcagct ggcatcatga agaaccctgt tgtggatgga
atagtggtga ctgacattgd cacaatgaca attgaccccc aagtggagaa aaagatggga
caggtgctac tcatagcagt agccgtctcc agcgccatac tgtcgcggac cgcctggggg
tggggggagg ctggggccct gatcacagct gcaacttcca ctttgtggga aggctctccg
aacaagtact ggaactcctc tacagccact tcactgtgta acatttttag gggaagttac
ttggctggag cttctctaat ctacacagta acaagaaacg ctggcttggt caagagacgt
gggggtggaa caggagagac cctgggagag aaatggaagg cccgcttgaa ccagatgtcg
gccctggagt tctactccta caaaaagtca ggcatcaccg aggtgtgcag agaagaggcc
cgccgcgccc tcaaggacgg tgtggcaacg ggaggccatg ctgtgtcccg aggaagtgca
aagctgagat ggttggtgga gcggggatac ctgcagccct atggaaaggt cattgatctt
ggatgtggca gagggggctg gagttactac gccgccacca tccgcaaagt tcaagaagtg
aaaggataca caaaaggagg ccctggtcat gaagaaccca tgttggtgca aagctatggg
tggaacatag tccgtcttaa gagtggggtg gacgtctttc atatggcggc tgagccgtgt
gacacgttgc tgtgtgacat aggtgagtca tcatctagtc ctgaagtgga agaagcacgg
acgctcagag tcctttccat ggtgggggat tggcttgaaa aaagaccagg agccttttgt
ataaaagtgt tgtgtccata caccagcact atgatggaaa ccctggagog actgcagcgt
aggtatgggg gaggactggt cagagtgcca ctctcccgca actctacaca tgagatgtac
tgggtctctg gagcgaaaag caacaccata aaaagtgtgt ccaccacgag ccagctcctc
ttggggcgca tggacgggcc caggaggcca gtgaaatatg aggaggatgt gaatctcggc
tctggcacgc gggctgtggt aagctgcgct gaagctccca acatgaagat cattggtaac
cgcattgaaa ggatccgcag tgagcacgcg gaaacgtggt tctttgacga gaaccaccca
tataggacat gggcttacca tggaagctat gaggccccca cacaagggtc agcgtcctct
ctaataaacg gggttgtcag gctcctgtca aaaccctggg atgtggtgac tggagtcaca
ggaatagcca tgaccgacac cacaccgtat ggtcagcaaa gagttttcaa ggaaaaagtg
gacactaggg tgccagatcc ccaagaaggc actcgtcagg ttatgagcat ggtctcttcc
tggttgtgga aagagctagg caaacacaaa cggccacgag tctgtaccaa agaagagttc
atcaacaagg ttcgtagcaa tgcagcatta ggggcaatat ttgaagagga aaaagagtgg
aagactgcag tggaagctgt gaacgatcca aggttctggg ctctagtgga caaggaaaga
gagcaccacc tgagaggaga gtgccagagt tgtgtgtaca acatgatggg aaaaagagaa
aagaaacaag gggaatttgg aaaggccaag ggcagccgcg ccatctggta tatgtggcta
ggggctagat ttctagagtt cgaagccctt ggattcttga acgaggatca ctggatgggg
agagagaact caggaggtgg tgttgaaggg ctgggattac aaagactcgg atatgtccta
gaagagatga gtcgcatacc aggaggaagg atgtatgcag atgacactgc tggctgggac
acccgcatca gcaggtttga tctggagaat gaagctctaa tcaccaacca aatggagaaa
gggcacaggg ccttggcatt ggccataatc aagtacacat accaaaacaa agtggtaaag
gtccttagac cagctgaaaa agggaagaca gttatggaca ttatttcgag acaagaccaa
agggggagcg gacaagttgt cacttacgct cttaacacat ttaccaacct agtggtgcaa
ctcattcgga gtatggaggc tgaggaagtt ctagagatgc aagacttgtg gctgctgcgg
aggtcagaga aagtgaccaa ctggctgcag agcaacggat gggataggct caaacgaatg
gcagtcagtg gagatgattg cgttgtgagg ccaattgatg ataggtttgc acatgccctc
aggttcttga atgatatggg gaaagttagg aaggacacac aagagtggaa accctcaact
ggatgggaoa actgggagga agttccgttt tgctcccacc acttcaacaa gctccatctc
aaggacggga ggtccattgt ggttccctgc cgccaccaag atgaactgat tggccgggcc
cgcgtctctc caggggcggg atggagcatc cgggagactg cttgcctagc aaaatcatat
gcgcaaatgt ggcagctcct ttatttccac agaagggacc tccgactgat ggccaatgoc
atttgttcat ctgtgccagt tgactgggtt ccaactggga gaactacctg gtcaatccat
ggaaagggag aatggatgac cactgaagac atgcttgtgg tgtggaacag agtgtggatt
gaggagaacg accacatgga agacaagacc ccagttacga aatggacaga cattccctat
ttgggaaaaa gggaagactt gtggtgtgga tctctcatag ggcacagacc gcgcaccacc
tgggctgaga acattaaaaa cacagtcaac atggtgcgca ggatcatagg tgatgaagaa
aagtacatgg actacctatc cacccaagtt cgctacttgg gtgaagaagg gtctacacct
ggagtgctgt aa

prM/E proteins include those having at least 80%, 82%, 85%, 87%, 90%, 92%, 95%, 97%, 99% or more amino acid sequence identity to the prM/E proteins encoded by SEQ ID NO:1, SEQ ID NO:2, SEQ ID NO:3, SEQ ID NO:5, SEQ ID NO:11, SEQ ID NO:12, or SEQ II) NO:13.

Capsid proteins include those having at least 80%, 82%, 85%, 87%, 90%, 92%, 95%, 97%, 99% or more amino acid sequence identity to the proteins encoded by one or more of SEQ ID NO:1, SEQ ID NO:2, SEQ ID NO:3, SEQ ID NO:5, SEQ NO:11, SEQ ID NO:12, or SEQ NO:13,

An exemplary intron/enhancer sequences useful in a vector include:

atcgcctggagacgccatccacgctgttttgacct
ccatagaagacaccgggaccgatccagcctccgcg
gccgggaacggtgcattggaacgcggattccccgt
gccaagagtgactcaccgtccggatctcagcaagc
aggtatgtactctccagggtgggcctggcttcccc
agtcaagactccagggatttgagggacgctgtggg
ctcttctcttacatgtaccttttgcttgcctcaac
cctgactatcttccaggtcaggatcccagagtcag
gggtctgtattttcctgctggtggctccagttcag
gaacagtaaaccctgctccgaatattgcctctcac
atctcgtcaatctccgcgaggactggggaccctgt
gacgaac

(SEQ ID NO:4), or a nucleotide sequence having at least 80%, 82%, 85%, 87%, 90%, 92%, 95%, 97%, 99% or more nucleotide sequence identity to SEQ ID NO:4.

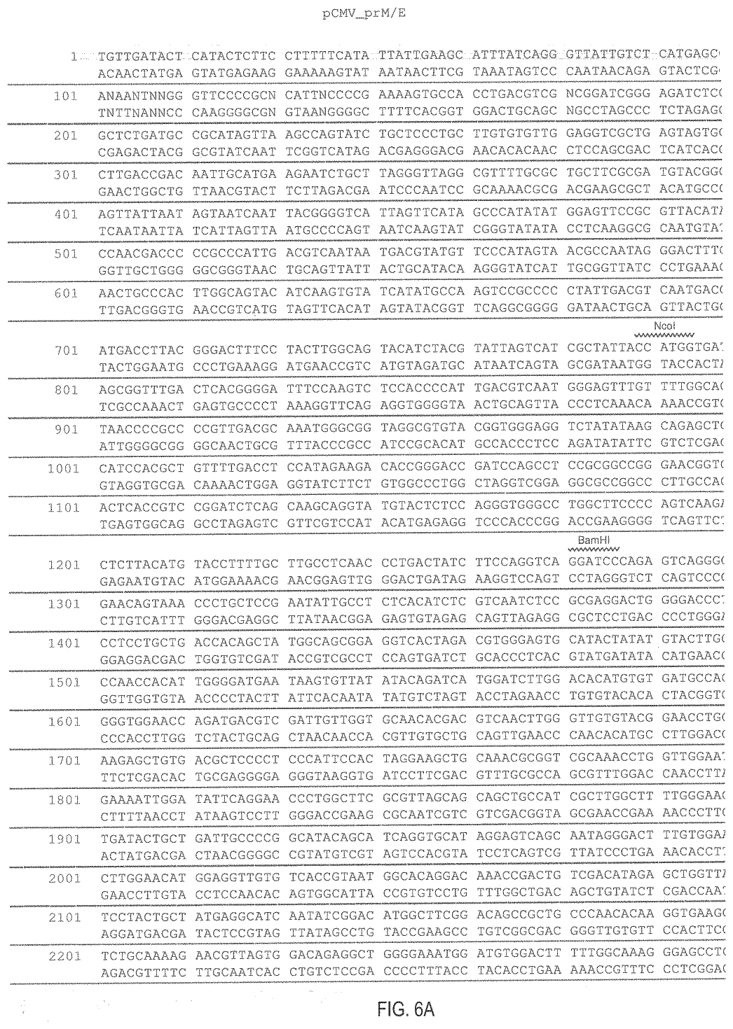

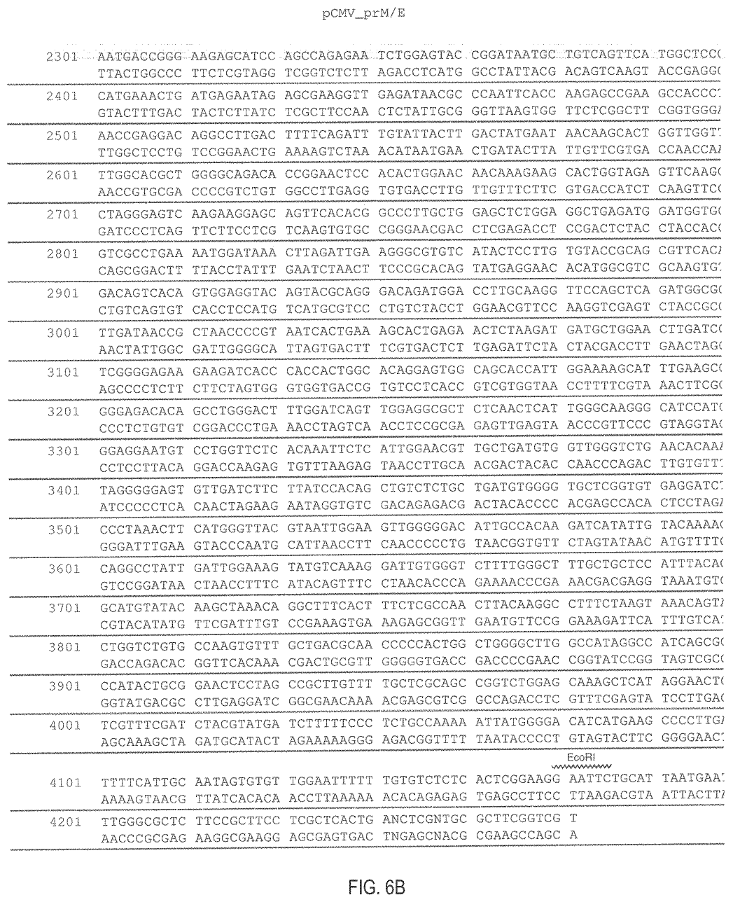

An exemplary vector sequence useful to produce VLPs is shown in FIG. 6 (SEQ ID NO:5).

An exemplary African lineage Zika isolate has the following nucleotide sequence (SEQ ID NO:11 which encodes SEQ NO:14; see Accession No. HQ234500 which is incorporated by reference herein):

atgaaaaacc caaagaagaa atccggagga ttccggattg
tcaatatgct aaaacgcgga gtagcccgtg taaacccctt
ggggggtttg aagaggctgc cggccggact cctgctgggc
catggaccca tcagaatggt ttcggcgata ctagccttct
tgagattcac agcaatcaag ccatcactgg gcctcatcaa
tagatggggt tccgtgggga agaaggaggc tatggaaata
ataaaaaagt tcaagaaaga tcttgctgcc atgttgagaa
taatcaatgc taggaaggag aggaagagac gtggagctga
tgccagcatc ggaatcgtca gcctcctgct gactacagtc
atggcagcag agatcactag acgcgggagt gcatactaca
tgtacttgga caggagcgat gctggtaagg ccatttcttt
cgttaccaca ctgggggtga acaaatgcca tgtgcagatc
atggacctcg ggcatatgtg tgacgccacc atgagttatg
agtgccccat gctggacgag ggagtggagc cagatgacgt
cgattgctgg tgcaacacga catcaacttg ggttgtgtac
ggaacctgtc atcataaaaa aggtgaagca cgacgatcca
gaagagccgt gacgcttcct tctcactcta caaggaagtt
gcaaacgcga tcgcagactt ggctagaatc aagagaatac
acaaagcacc tgatcaaggt tgagaattgg atattcagga
accccgggtt tgcgctagtg gctgtagcta ttgcctggct
cctgggaagc tcgacgagcc aaaaagtcat acacttggtc
atgatattgt tgattgcccc ggcatacagt atcaggtgca
taggagttag caatagagac ttcgtggagg gcatgtcagg
tgggacctgg gttgatgttg tcttggaaca tggaggttgt
gtcaccgtga tggcacagga caagccaaca gttgacatag
agttggtcac gacaacggtt agcaacatgg ccgaggtgag
atcctactgc tacgaggcat caatatcgga catggcttcg
gacagtcgct gcccaacaca aggtgaagcc taccttgaca
agcagtcaga cactcaatat gtctgtaaaa gaacattggt
ggacagaggt tggggaaatg ggtgtggact ttttggcaag
gggagcttgg tgacgtgtgc caagtttaca tgctccaaga
aaatgacagg gaagagcatc cagccggaga acttggagta
ccggataatg ctatcagtgc atggatccca gcacagtggg
atgattgtga atgacgaaaa cagagcaaaa gtcgaggtta
cacccaattc accaagagca gaagcaacct tgggaggttt
tggaagcctg ggacttgatt gtgaaccaag gacaggcctt
gacttttcag atctgtatta cctgaccatg aacaataagc
attggttggt gcacaaagag tggtctcatg acatcccatt
accttggcat tctggtgcag acactgaaac tccacactgg
aacaacaaag aggcactggt ggagttcaag gacgcccacg
ccaagaggca aactgttgtg gttctgggga gccaagaagg
agccgttcac acggctctcg ctggagctct ggaggctgag
atggatggtg cgaagggaag gctatcctca ggccatttga
aatgccgcct aaaaatggac aagcttaggt tgaagggtgt
gtcatattcc ctgtgtaccg cagcgttcac attcaccaag
gttccagctg aaacattgca tggaacagtc acagtggagg
tgcagtatgc agggagggat ggaccctgca aggtcccagc
ccagatggcg gtggacatgc agaccctgac cccagttgga
aggctgataa cggctaaccc tgtgatcact gaaagcactg
agaattcaaa gatgatgttg gagctcgacc caccatttgg
ggattcttac attgtcatag gagtcgggga caagaaaatc
acccatcact ggcatcggag tggtagcatc atcggaaagg
catttgaagc cactgtgaga ggcgccaaga gaatggcagt
cttgggagac acagcctggg actttggatc agttgggggt
gtgtttaact cattgggcaa gggtattcac cagatctttg
gagcagcttt caaatcactg ttcggaggaa tgtcctggtt
ctcacagatc ctcataggca cactgttggt gtggttgggt
ctgaacacaa agaatggatc tatctccctc acatgcttgg
ccttgggagg agtgatgatc ttcctttcca cggctgtttc
tgctgatgtg gggtgttcgg tggacttctc aaaaaaggaa
acgagatgtg gcacgggggt gttcatctac aatgacgttg
aagcctggag ggatcgatac agataccatc ctgactcccc
ccgcagattg gcagcagctg ctaagcaggc ttgggaagag
gggatttgtg ggatctcctc cgtttcgaga atggaaaaca
ccatgtggaa atcagtggaa ggggagctta atgcgatcct
agaggagaat ggagtccaac tgacagttgt agtggggtct
gtaaaaaacc ccatgtggag aggtccacga agattgccag
tgcccgtaaa tgagctgccc catggctgga aagcctgggg
gaaatcgtac tttgttaggg cggcaaagac caacaacagt
tttgttgtcg acggtgacac actgaaggaa tgtccgctca
aacatagagc atggaatagc ttccttgtgg aggatcacgg
gtttggggtc ttccacacca gtgtttggct gaaggtcaga
gaggactatt cattagagtg tgacccagcc gtcataggaa
cagctgtcaa gggaaaggag gctgcacaca gtgatctagg
ctattggatt gagagtgaaa agaatgacac atggaggctg
aagagggctc atctgattga gatgaagaca tgtgagtggc
caaagtctca cacactgtgg acagatggag tggaagaaag
tgatctgatc atacccaagt ccttagctgg tccactcagc
caccacaaca ccagagaggg ttatagaact caagtgaaag
ggccatggca tagtgaagag ctgaaatccc ggtttgagga
atgcccaggc accaaggttc atgtggagga gacatgcgga
actagaggac catctctaag atcaaccact gcaagtggaa
gggccataga ggaatggtgc tgtagggaat gcacaatgcc
tccactatcg ttccgggcaa aagacggctg ctggtatgga
atggagataa ggcccagaaa ggaaccagag agcaacttag
tgaggtctat ggtgacagca ggatcaaccg atcacatgga
tcacttctct cttggagtgc ttgtgattct actcatggtg
caggaaggtt tgaagaagag aatgaccaca aagatatcaa
tgagcacacc aatggcaatg ctggtagcca tggtcttggg
aggattctca atgagtgacc tggctaagct tgtgatcctg
atgggtgcca ctttcgcaga aatgaacact ggaggagatg
tggctcactt ggcattggta gcggcattta aagtcagacc
agccttgttg gtttccttca tcttcagagc caactggaca
ccccgtgaga gcatgctgct agccctggct tcgtgtctcc
tgcagactgc gatttccgct cttgaaggcg agctgatggt
cctcgttaat ggatttgctt tggcctggtt ggcaatacga
gcaatggccg tgccacgcac tgataacatc gctctagcaa
ttctggccgc tctaacacca ttagccagag gcacactgct
tgtggcatgg agagcgggcc tgccactctg tggagggttc
atgctcattt ccctgaaagg gaaaggtagt gtgaagaaga
acctgccact tgtcatggcc ttggggttga ccgctgtgag
gatagtggac cccattaatg tggtaggact actgttactg
acaaggagtg ggaaacggag ctggccccct agtgaagtgc
ttacagctgt cggcctgata tgtgcactgg ccggagggtt
tgccaaggca gacatagaga tggctgggcc catggctgca
gtaggcctgc taattgtcag ttatgtggtc acgggaaaga
gtgtggacat gtacattgaa agagcaggtg atattacatg
ggaaaaagac gcggaagtca ctggaaacag tcctcggctt
gacgtggcac tagatgagag tggtgatttc tctttggtag
aggaggatgg cccacccatg agagagatca tactcaaggt
ggtcctgatg gccatctgtg gcatgaaccc aatagccata
cccttcgctg caggagcgtg gtatgtgtat gtaaagactg
ggaaaaggag cggtgccctc tgggacgtgc ctgctcccaa
agaagtaaaa aagggagaga ctacagatgg agtgtacaga
gttatgactc gcagactgct gggttcaaca caggttggag
tgggagtcat gcaagaggga gtcttccata ccatgtggca
cgtcacaaaa ggagccgcat tgaggagcgg tgaaggaaga
cttgatccat actgggggga cgtcaagcag gacctggtgt
catattgtgg gccgtggaag ttggatgcag cctgggatgg
actaagtgag gtgcagcttt tggccgtacc ccccggagag
agggctaaaa acattcagac tctgcctgga atatttaaga
caaaggatgg ggacatcgga gcagttgctc tagactaccc
tgcaggaacc tcaggatctc cgatcctaga caaatgcgga
agagtgatag gactttatgg caatggggtt gtgatcaaga
atggaagcta tgttagtgcc ataacccagg gaaaaaggga
ggaggagact ccggttgagt gctttgaacc ctcgatgctg
aggaagaagc agctaacagt cttggatctg catccaggag
ccgggaaaac caggagggtt cttcctgaaa tagtccgtga
agccataaag aagagacttc gcacagtgat cttagcacca
accagggttg ttgctgctga gatggaggaa gccctaagag
gacttccggt gcgttacatg acaacagcag tcaacgtcac
ccattctggg acagaaatcg ttgatttgat gtgccatgcc
accttcactt cacgcctact acaaccaatc agagtcccca
actacaacct ttatatcatg gatgaggctc atttcacaga
tccttcaagc atagctgcaa gaggatacat atcaacaagg
gttgaaatgg gcgaggcggc tgctatcttc atgactgcta
caccaccagg aacccgcgat gcgtttccag attccaactc
accaatcatg gacacagaag tggaagtccc agagagagcc
tggagctcag gctttgactg ggtgacggac cattctggaa
aaacaatttg gtttgttcca agtgtgagaa acggaaatga
aatcgcagcc tgtctgacaa aggctggaaa gcgggttata
cagctcagca ggaagacttt tgagacagag tttcagaaga
caaaaaatca agagtgggac tttgtcataa caactgacat
ttcagagatg ggtgccaact tcaaggctga ccgggtcata
gattccagga gatgcctaaa gccagtcata cttgatggtg
agagagtcat cctggctggg cctatgcccg tcacgcacgc
cagtgctgct cagaggagag gacgtatagg caggaacccc
aacaaacctg gagatgagta tatgtatgga ggtgggtgtg
cagagactga tgaagaccat gcacactggc ttgaagcaag
aatgcttctc gacaacattt acctccagga tggccccata
gcctcgctct atcggcctga ggctgacaag gttgccgcca
ttgagggaga gttcaagctg aggacagagc aaaggaagac
ctttgtggaa ctcatgaaga gaggagacct tcccgtttgg
ctggcctatc aagtagcatc tgccggaata acttacacag
acagaagatg gtgctttgat ggcactacca acaacaccat
aatggaagac agtgtaccag cagaggtgtg gaccaagtat
ggagagaaga gagtgctcaa accgaggtgg atggatgcca
gggtctgttc agatcatgcg gctttgaagt cgttcaaaga
atttgccgct gggaagagag gagcggcttt gggagtaatg
gatgccctag gaacattgcc aggacacatg acagagaggt
ttcaggaagc cattgacaat ctcgctgtgc tcatgcgagc
agagactgga agtaggccct acaaagcagc ggcagctcaa
ctgccggaga ccctagagac cattatgctc ttgggtttat
tgggaacagt ttcgctaggg atcttctttg tcttgatgcg
gaacaagggc atcgggaaga tgggcttcgg aatggtaacc
cttggggcca gcgcatggct catgtggctt tcggaaattg
aaccagccag aatcgcatgt gtcctcattg tcgtgtttct
gttactggtg gtgctcatac ctgagccaga gaagcaaaga
tctccccagg acaatcaaat ggcaatcatc atcatggtgg
cagtgggcct tctgggtttg ataactgcaa acgaactcgg
atggctggaa agaacaaaaa gtgatatagc tcatctaatg
ggaaggaaag aagaggggac aaccgtagga ttctcaatgg
atattgatct gcggccagcc tccgcctggg ctatttatgc
cgcattgaca actctcatca ccccagccgt ccaacatgcg
gtgaccacct catacaacaa ctactccctg atggcgatgg
ccacacaagc tggagtgctg tttggcatgg gcaaagggat
gccattttat gcatgggact ttggagtccc gctgctaatg
atgggttgtt actcacaatt aacacccctg accctgatag
tggccatcat tctgcttgtg gcacactaca tgtatttgat
cccaggtttg caggcagcag cagcacgtgc cgcccagaag
aggacagcag ctggcatcat gaagaatccc gttgttgatg
gaatagtggt gactgacatt gacacaatga caattgaccc
ccaagtggag aagaagatgg gacaagtgtt actcatagca
gtagctgcct ccagtgccgt gctgctgcgg accgcttggg
gatgggggga ggctggggct ctgatcacag cagcaacctc
caccttatgg gaaggctctc caaacaaata ctggaactcc
tctacagcca cttcactgtg caatatcttc agaggaagtt
atttggcagg ggcttccctt atttacacag tgacaagaaa
tgccggtctg gttaagagac gtggaggtgg aacgggagag
actctgggag agaagtggaa agcccgcctg aaccagatgt
cggctttgga gttctattct tacaaaaagt caggcatcac
cgaagtgtgt agggaggagg cacgccgcgc cctcaaggat
ggagtggcca caggaggaca tgctgtatcc cggggaagcg
caaagcttag atggttggta gagagaggat acctgcagcc
ccatggaaag gttgttgacc tcggatgtgg cagagggggc
tggagttatt acgctgccac catccgtaaa gtgcaggagg
tcagaggata cacaaaggga ggtcctggtc atgaagaacc
catgctggtg caaagctatg ggtggaacat agttcgcctc
aagagtggag tggacgtctt tcacatggcg gctgagccgt
gtgacacctt gctgtgtgac attggcgagt catcgtccag
tcctgaagtg gaagagacgc gaacactcag agtgctctcc
atggtgggag actggctcga gaaaagacca ggggccttct
gcataaaggt gctgtgccca tacaccagta ctatgatgga
gaccatggag cgactgcaac gtaggtatqq gggaggattg
gtcagagtgc cattgccccg caactccaca catgagatgt
attgggtctc tggagccaaa agtaacatca taaagagtgt
gtccaccaca agtcagctcc tcttgggacg catggatggg
cctaggaggc cagtgaaata tgaagaggat gtgaacctcg
gctcaggcac acgagctgtg gcaagctgtq ctgaggctcc
caacatgaag atcattggta ggcgcattga gagaatccgc
aatgaacatg cagagacacg gttctttgat gaaaaccacc
catacaggac atgggcctac catgggagct acgaagcccc
cacgcagggg tcagcgtcat ccctcgtgaa cggggttgtt
agactcctgt caaagccctg ggatgtggtg actggagtca
caggaatagc tatgactgac accacgccat acggccaaca
aagagtcttc aaagaaaagg tggacactag ggtgccagac
ccccaagaag gcacccgccg agtaatgaac atggtctcgt
cttggctatg gaaggagctg ggaaaacgca agcggccacg
tgtctgcacc aaagaagagt tcatcaataa ggtgcgcagc
aatgcagcac tgggagcaat atttgaagag gaaaaagaat
ggaagacagc tgtagaagct gtgaatgatc cgagattttg
ggctctagtg gacaaggaaa gagaacacca cctgagagga
gagtgtcaca gctgtgtgta caacatgatg ggaaaaagag
aaaagaagca aggagaattc gggaaagcaa aaggcagccg
cgcaatctgg tacatgtggt tgggagccag atttctggag
tttgaggctc ttggattctt gaatgaggac cattggatgg
gaagagaaaa ctcaggaggt ggcgttgaag ggctaggact
gcaaaggctt ggatacattc tagaagaaat gaaccgggcg
ccaggaggaa agatgtatgc agatgacacc gctggctggg
atacccgtat tagcaggttt gatctggaga atgaagccct
gatcactaac cagatggaag aagggcacag agctctggcg
ttggccgtga ttaaatacac ataccaaaac aaagtggtga
aggttctcag accagctgaa ggagggaaaa cagtcatgga
catcatctca agacaagacc agagagggag cggacaagtt
gttacttatg ccctcaacac attcaccaac ctggtggtgc
agcttatccg gaacatggag gctgaggagg tgctagagat
gcatgatcta tggctgttga ggaagccaga gaaagtgacc
agatggttgc agagcaatgg atgggacaga ctcaaacgaa
tggcagtcag tggagatgac tgcgttgtaa agccaattga
tgataggttt gcacatgccc tcaggttctt gaatgacatg
ggaaaagtta ggaaagacac acaggaatgg aaaccctcga
ctggatggag caattgggaa gaagtcccgt tctgttccca
ccacttcaac aagctgcacc tcaaggatgg gagatccatt
gtggtcccct gccgccacca agatgaactg attggccgag
cccgtgtctc accaggggca ggatggagca tccgggagac
tgcctgtctt gcaaaatcat atgcccagat gtggcagctt
ctttatttcc acagaagaga cctccgactg atggccaatg
ccatctgttc ggccgtgcca gccgactggg tcccaactgg
gagaaccacc tggtcaatcc atggaaaggg agaatggatg
actaatgagg acatgctcat ggtgtggaat agagtgtgga
ttgaggagaa cgaccacatg ggggacaaga cccctgtaac
aaaatggaca gacattccct atttgggaaa aagggaggac
ttatggtgtg gatcccttat agggcacaga cttcgcacca
cttgggctga gaacatcaaa gacacagcca acatggtgcg
taggatcata ggtgatgaag aaaggtacat ggactaccta
tccacccagg tacgctactt gggtgaggag gggtccacac
ctggagtgct g

An exemplary Asian lineage Zika isolate has the following sequence (SEQ ID NO:12 which encodes SEQ ID NO:15; see Accession No. HQ234499 which is incorporated by reference herein):

ATGAAAAACC AAAAAAGAA TCCGGAGGA TCCGGATTG
TCAATATGCT AAACGCGGA TAGCCCGTG GAGCCCCTT
TGGGGGCTTG AGAGGCTAC AGCTGGACT CTGCTGGGT
CATGGACCCA CAGGATGGT TTGGCGATA TAGCCTTCT
TGAGATTCAC GCAATCAAG CATCACTGG TCTCATCAA
TAGATGGGGT CCGTGGGGA AAAAGAGGC ATGGAAATA
ATAAAGAAGT CAAGAAAGA CTGGCTGCC TGCTGAGAA
TAATCAATGC AGGAAGGAG AGAAGAGAC TGGCGCAGA
CACCAGTGTC GAATTGTTG CCTCCTGCT ACCACAGCC
ATGGCAGTGG GGTCACCAG CGTGGGAGT CATACTATA
TGTACTTAGA AGAAGCGAT CTGGGGAGG CATATCTTT
TCCAACCACA TGGGGGTGA TAAGTGTTA ATACAGATC
ATGGATCTTG ACACATGTG GATGCCACA TGAGCTATG
AATGCCCTAT TTGGATGAG GGGTAGAAC AGATGACGT
CGATTGCTGG GCAACACGA ATCGACTTG GTTGTGTAC
GGAACCTGCC TCACAAAAA GGTGAGGCA GGAGATCTA
GAAGAGCTGT ACGCTCCCC CTCATTCCA TAGGAAGCT
GCAAACGCGG CGCAGACCT GTTGGAATC AGAGAATAC
ACAAAGCACT GATCAGAGT GAAAATTGG TATTCAGGA
ACCCTGGCTT GCGTTGGCA CAGCTGCCA TGCTTGGCT
TTTGGGAAGC CAACGAGCC AAAAGTCAT TACTTGGTC
ATGATACTGT GATTGCCCC GCATACAGT TCAGGTGCA
TAGGAGTCAG AATAGGGAT TTGTGGAAG TATGTCAGG
TGGGACCTGG TTGATGTTG CTTGGAACA GGAGGTTGT
GTTACCGTAA GGCACAGGA AAGCCAACT TTGATATAG
AGTTGGTCAC ACAACGGTT GCAACATGG GGAGGTAAG
ATCCTACTGC ACGAGGCAT AATATCGGA ATGGCTTCG
GACAGCCGCT CCCAACACA GGTGAAGCC ACCTTGACA
AGCAGTCAGA ACTCAATAT TTTGCAAAA AACGTTAGT
GGACAGAGGT GGGGAAATG ATGTGGACT TTTGGCAAA
GGGAGCCTGG GACATGCGC AAGTTTGCA GCTCCAAGA
AAATGACTGG AAGAGCATC AGCCAGAGA CCTGGAGTA
CCGGATAATG TGTCAGTTC TGGCTCCCA CACAGTGGG
ATGATTGTTA TGACANAGG CATGAAACT ATGAGAATA
GAGCGAAGGT GAGATAACG CCAATTCAC AAGAGCCGA
AGCCACCCTG GAGGTTTTG AAGCCTAGG CTTGATTGT
GAACCGAGGA AGGCCTTGA TTTTCAGAT TGTATTACT
TGACTATGAA AACAAGCAT GGTTGGTGC CAAGGAGTG
GTTCCATGAC TTCCACTAC TTGGCATGC GGGGCAGAC
ACCGGAACTC ACATTGGAA AACAAAGAA CATTGGTAG
AGTTCAAGGA GCACATGCC AAAGGCAAA TGTCGTGGT
TCTAGGGAGT AAGAAGGAG CGTTCACAC GCTCTTGCT
GGAGCCCTGG GGCTGAGAT GATGGTGCA AGGGAAGGC
TGTCCTCTGG CACTTGAAA GTCGCTTGA AATGGACAA
ACTTAGATTG AGGGCGTGT ATACTCCTT TGTACCGCG
GCGrrCACAT CACCAAGAT CCGGCTGAA CGCTGCATG
GGACAGTCAC GTGGAGGTA AGTATGCAG GACAGATGG
ACCCTGCAAG TTCCAGCTC GATGGCGGT GATATGCAA
ACTCTGACCC AGTTGGGAG TTGATAACC CTAACCCTG
TGATCACTGA AGCACTGAG ATTCAAAGA GATGTTGGA
ACTTGACCCA CATTTGGGG TTCTTACAT GTCATAGGA
GTTGGGGATA GAAGATCAC CACCACTGG ACAGGAGTG
GCAGCACCAT GGAAAAGCA TTGAAGCCA TGTGAGAGG
CGCCAAGAGA TGGCAGTCT GGGAGACAC GCCTGGGAC
TTTGGATCAG CGGAGGTGC CTCAACTCA TGGGGAAGG
GCATCCATCA ATTTTTGGA CAGCTTTCA ATCATTGTT
TGGAGGAATG CCTGGTTCT ACAAATCCT ATAGGAACG
TTGCTGGTGT GTTGGGTCT AACACAAAG ATGGATCTA
TTTCCCTTAC TGCTTGGCC TAGGGGGAG GTTGATCTT
CCTATCTACA CCGTCTCTG TGATGTGGG TGTTCGGTG
GACTTCTCAA GAAGGAAAC AGATGCGGT CGGGGGTGT
TCGTCTATAA GACGTTGAA CCTGGAGGG CAGGTACAA
GTACCATCCT ACTCCCCTC TAGATTGGC GCAGCAGTC
AAGCAGGCCT GGAAGATGG ATCTGTGGG TCTCCTCTG
TTTGAAGAAT GAAAACATT TGTGGAGAT AGTAGAAGG
GGAGCTCAAC CAATTCTGG AGAGAATGG GTTCAACTG
ACGGTCGTTG GGGATCTGT AAAAACCCC TGTGGAGAG
GTCCGCAGAG TTGCCTGTG CTGTGAATG GCTGCCCCA
CGGTTGGAAG CCTGGGGGA ATGGTACTT GTCAGGGCA
GCAAAGACCA CAACAGCTT GTTGTGGAT GTGACACAC
TGAAGGAATG CCGCTCAAA ACAGAGCAT GAACAGCTT
TCTTGTGGAG ATCACGGGT CGGGGTATT CACACTAGT
GTCTGGCTTA AGTCAGAGA GATTACTCA TAGAGTGTG
ATCCAGCCGT ATAGGAACA CTGCTAAGG AAAGGAGGC
CGTGCACAGT ATCTAGGCT CTGGATTGA AGTGAAAAG
AACGACACAT GAGGCTGAA AGGGCTCAC TGATCGAGA
TGAAAACATG GAATGGCCA AGTCCCACA ACTGTGGAC
AGATGGAATA AAGAAAGTG TCTGATCAT CCTAAGTCT
TTAGCTGGGC ACTCAGCCA CACAACACC GAGAGGGCT
ACAGGACTCA GTGAAAGGG CGTGGCATA TGAAGAGCT
TGAAATCCGG TTGAGGAAT TCCAGGCAC AAGGTCCAC
GTGGAGGAAA ATGTGGAAC AGAGGACCG CCCTGAGAT
CAACCACTGC AGCGGAAGG TGATCGAGG ATGGTGCTG
CAGGGAATGC CAATGCCCC ATTGTCGTT CGGGCAAAA
GATGGCTGTT GTATGGAAT GAGATAAGG CCAGGAAGG
AACCAGAGAG AACCTAGTA GGTCAATGG GACTGCAGG
ATCAACTGAT ACATGGATC CTTCTCCCT GGAGTGCTT
GTGATTCTGC CATGGTGCA GAAGGGCTG AGAAGAGAA
TGACCACAAA ATCATCATA GCACATCAA GGCAGTGTT
GGTAGCTATG TCCTGGGAG ATTTTCAAT AGTGACTTG
GCTAAGCTTG AATTCTGAT GGTGCCACC TCGCGGAAA
TGAACACTGG GGAGATGTA CTCATCTGG GCTGATAGC
GGCATTCAAA TCAGACCCG GTTGCTGGT TCTTTCATC
TTCAGAGCCA TTGGACACC CGTGAGAGC TGCTGCTGG
CCTTGGCCTC TGCCTTCTG AAACTGNGA CTCCGCCCT
GGAAGGCGAC TGATGGTTC CATCAATGG TTTGCTTTG
GCCTGGTTGG AATACGAGC ATGGCTGTT CACGCACTG
ACAACATCAC TTGGCAATC TGGCTGCTC GACACCACT
GGCCCGAGGC CACTGCTTG AGCGTGGAG GCAGGCCTT
GCTACTTGTG GGGGTTCAT CTCCTCTCT TGAAGGGGA
AAGGTAGTGT AAGAAGAAC TACCATTTG CATGGCCTT
GGGACTAACC CTGTGAGGC GGTTGACCC ATCAACGTG
GTGGGACTGC GTTGCTCAC AGGAGTGGG AGCGGAGCT
GGCCCCCTAG GAAGTACTC CAGCTGTTG CCTGATATG
TGCACTGGCC GAGGGTTCG CAAAGCAGA ATAGAGATG
GCTGGGCCCA GGCTGCAGT GGCCTGCTA TTGTTAGTT
ACGTGGTCTC GGAAAGAGT TGGACATGT CATTGAAAG
AGCAGGTGAC TCACATGGG AAAAGATGC GAAGTTACT
GGAAACAGCC CCGGCTCGA GTGGCACTA ATGAGAGTG
GTGATTTCTC CTGGTGGAG ATGATGGTC CCCCATGAG
AGAGATCATA TCAAGGTGG CCTGATGAC ATCTGTGGC
ATGAACCCAA AGCCATACC TTTGCAGCT GAGCGTGGT
ATGTGTATGT AAGACTGGA AGAGGAGTG TGCTCTATG
GGATGTGCCT CTCCCAAGG AGTAAAAAA GGGGAGACC
ACAGATGGAG GTATAGAGT ATGACTCGC GACTGCTAG
GTTCAACACA GTTGGAGTG GAGTCATGC AGAGGGGGT
CTTCCACACT TGTGGCACG CACAAAAGG TCCGCGCTG
AGGAGCGGTG AGGGAGACT GATCCATAC GGGGAGATG
TTAAGCAGGA CTGGTGTCA ACTGTGGCC GTGGAAGCT
AGATGCCGCT GGGACGGAC CAGCGAGGT CAGCTTTTG
GCCGTGCCCC CGGAGAGAG GCGAGGAAC TCCAGACTC
TGCCCGGAAT TTCAAGACA AGGATGGGG CATCGGAGC
AGTTGCTCTG CTTACCCAG AGGAACTTC GGATCTCCG
ATCCTAGACA GTGTGGGAG GTGATAGGA TCTATGGCA
ATGGGGTCGT ATCAAAAAT GAAGTTATG TAGTGCCAT
CACCCAAGGG GGAGGGAGG AGAGACTCC GTTGAATGC
TTCGAACCTT GATGCTGAA AAGAAGCAG TAACTGTCT
TGGATCTGCA CCTGGAGCT GGAAAACCA GAGAGTTCT
TCCTGAAATA TCCGTGAAG CATAAAAAC AGACTCCGC
ACGGTGATCC GGCTCCAAC AGGGTTGTC CTGCTGAAA
TGGAGGAAGC CTTAGAGGG TTCCAGTGC TTACATGAC
AACAGCAGTT ATGTCACCC CTCTGGGAC GAAATCGTT
GATTTAATGT CCATGCCAC TTCACTTCA GCCTACTAC
AACCCATTAG GTCCCCAAC ACAATCTTT CATTATGGA
TGAGGCCCAC TCACAGATC CTCAAGTAT GCAGCAAGA
GGATACATAT AACAAGGGT GAGATGGGC AGGCGGCTG
CCATCTTCAT ACCGCCACA CACCAGGAA CCGCGACGC
ATTTCCGGAC CTAACTCAC AATCATGGA ACAGAAGTG
GAAGTCCCAG GAGAGCCTG AGCTCAGGC TTGATTGGG
TGACGGATCA TCTGGAAAA CAGTTTGGT TGTTCCAAG
CGTGAGGAAC GCAACGAGA CGCGGCTTG CTGACAAAA
GCTGGAAAAC GGTCATACA CTCAGCAGA AGACTTTTG
AGACAGAGTT CAGAAAACA AAAATCAAG GTGGGACTT
CGTCGTAACA CTGACATCT AGAGATGGG GCCAACTTC
AAAGCTGACC GGTCATAGA TCCAGGAGA GCCTGAAGC
CGGTCATACT GATGGCGAG GAGTCATTC GGCTGGACC
CATGCCTGTC CACATGCCA CGCTGCCCA AGGAGGGGG
CGCATAGGCA GAATCCCAA AAACCTGGA ATGAGTATA
TGTATGGAGG GGGTGCGCA AGACTGATG AGACCATGC
ACACTGGCTT AAGCAAGAA GCTTCTTGA AACATTTAC
CTCCAAGATG CCTCATAGC TCGCTCTAT GACCTGAGG
CCGATAAGGT GCAGCCATT AGGGAGAGT CAAGCTTAG
GACGGAGCAA GGAAGACCT TGTGGAACT ATGAAAAGA
GGAGATCTTC TGTTTGGCT GCCTATCAG TTGCATCTC
CCGGAATAAC TACACAGAT GAAGATGGT TTTTGATGG
CACGACCAAC ACACCATAA GGAAGACAG GTGCCGGCA
GAGGTGTGGA CAGATACGG GAGAAAAGA TGCTCAAAC
CGAGGTGGAT GACGCCAGA TTTGTTCAG TCATGCGGC
CCTGAAGTCA TCAAAGAAT TGCCGCTGG AAAAGAGGA
GCGGCCTTTG AGTGATGGA GCCCTGGGA CACTGCCAG
GACACATGAC GAGAGGTTT AGGAAGCCA TGACAACCT
CGCTGTGCTC TGCGGGCAG GACTGGAAG AGGCCCTAC
AAAGCCGCGG GGCCCAATT CCGGAGACC TAGAGACCA
TCATGCTTTT GGTTTGCTG GAACAGTCT GCTGGGAAT
CTTCTTTGTC TGATGCGGA CAAGGGCAT GGGAAGATG
GGCTTTGGAA GGTGACCCT GGGGCTAGT CATGGCTTA
TGTGGCTCTC GAAATTGAG CAGCCAGAA TGCATGTGT
CCTCATTGTC TGTTTCTAT GCTGGTGGT CTCATACCT
GAGCCAGAAA GCAGAGATC CCCCAGGAC ACCAAATGG
CAATTATCAT ATGGTAGCA TGGGTCTTC GGGCTTGAT
AACCGCCAAT AACTCGGAT GTTGGAGAG ACAAAAAGT
GACCTAGGCC TCTAATGGG AGGAGAGAG AGGGGGCAA
CCATGGGATT TCAATGGAC TTGACTTGC GCCAGCCTC
AGCTTGGGCT TCTATGCCG TCTGACAAC CTCATCACC
CCAGCCGTCC ACATGCGGT ACCACTTCA ACAACAACT
ACTCCTTAAT GCGATGGCC CGCAAGCCG AGTGTTGTT
TGGCATGGGC AAGGGATGC ATTCTATGC TGGGACTTC
GGAGTCCCGC GCTAATGAT GGTTGCTAC CACAATTAA
CACCCTTGAC TTAATAGTG CCATCATTC GCTCGTGGC
GCACTACATG ACTTGATCC AGGTCTACA GCAGCAGCG
GCGCGCGCTG CCAGAAGAG ACGGCAGCT GCATCATGA
AGAACCCTGT GTGGATGGA TAGTGGTGA TGACATTGA
CACAATGACA TTGACCCCC AGTGGAGAA AAGATGGGA
CAAGTGCTAC CATAGCAGT GCCATCTCC GTGCCGTTC
TGCTGCGCAC GCCTGGGGG GGGGGGAGG TGGGGCCCT
GATCACAGCC CAACTTCCA TTTGTGGGA GGCTCTCCG
AATAAATACT GAACTCCTC ACAGCCACT CACTGTGTA
ACATTTTTAG GGAAGTTAC TGGCTGGAG TTCTCTTAT
TTACACAGTA CAAGAAACG TGGCCTGGT AAGAGACGT
GGAGGTGGAA GGGAGAGAC CTGGGGGAG AATGGAAGG
CCCGCCTGAA CAGATGTCG CCCTGGAGT TTACTCCTA
CAAAAAGTCA GCATCACCG AGTGTGCAG GAAGAAGCC
CGCCGCGCCC CAAGGACGG GTGGCAACA GAGGCCATG
CTGTGTCCCG GGAAGCGCA AGCTTAGAT GTTGGTGGA
GAGAGGATAC TGCAGCCCT TGGAAAGGT ATTGATCTT
GGATGTGGCA AGGGGGCTG AGTTACTAC CCGCCACCA
TCCGCAAAGT CAAGAGGTG AAGGATACA AAAGGGAGG
CCCTGGTCAT AAGAACCCA GTTGGTGCA AGCTATGGA
TGGAACATAG CCGTCTTAA AGTGGGGTG ACGTCTTTC
ACATGGCGGC GAGTCGTGT ACACTTTGC GTGTGACAT
AGGTGAGTCA CATCTAGTC TGAAGTGGA GAAGCACGG
ACGCTCAGAG ACTCTCCAT GTGGGGGAT GGCTTGAAA
AAAGACCAGG GCCTTTTGT TAAAGGTGT GTGCCCATA
CACCAGCACC TGATGGAAA CCTAGAGCG CTGCAGCGT
AGGTATGGGG AGGACTGGT AGAGTGCCA TCTCCCGCA
ACTCTACACA GAGATGTAC GGGTCTCTG AGCGAAAAG
CAACATCATA AAAGTGTGT CACCACGAG CAGCTCCTC
TTGGGACGCA GGACGGGCC AGGAGGCCA TGAAATATG
AGGAGGATGT AATCTCGGC CCGGCACGC AGCTGTGGC
AAGCTGCGCC AAGCTCCCA CCTGAAGAT ATTGGTAAC
CGCGTTGAGA GATCCGCAG GAGCATGCG AAACGTGGT
TCTTTGATGA AACCACCCA ACAGGACAT GGCTTACCA
TGGGAGCTAC AGGCCCCTA ACAAGGGTC GCGTCTTCT
CTCATAAACG GGTTGTCAG CTCCTGTCA AGCCCTGGG
ATGTGGTGAC GGAGTCACA GAATAGCCA GACCGACAC
CACACCGTAT GCCAGCAAA AGTTTTCAA GAAAAAGTG
GACACTAGGG GCCAGACCC CAGGAAGGC CTCGTCAGG
TGATGAACAT GTCTCTTCC GGCTATGGA GGAGCTAGG
TAAACACAAA GGCCACGAG TTGCACCAA GAAGAGTTC
ATCAATAAGG TCGCAGCAA GCAGCACTG GGGCAATAT
TTGAAGAGGA AAAGAATGG AGACTGCAG GGAAGCTGT
GAACGATCCA GGTTCTGGG CCTAGTGGA AAGGAAAGA
GAGCACCACT GAGAGGAGA TGTCAGAGC GTGTGTACA
ACATGATGGG AAAAGAGAA AGAAGCAAG GGAATTTGG
AAAGGCCAAG GCAGCCGCG CATTTGGTA ATGTGGCTA
GGGGCTAGAT TCTAGAGTT GAAGCCCTT GATTCTTGA
ACGAGGATCA TGGATGGGG GAGAGAATT AGGAGGTGG
TGTTGAAGGG TGGGATTAC AAGACTTGG TATGTTCTA
GAAGAAATGA CCGCACACC GGAGGAAAG TGTATGCAG
ATGATACCGC GGCTGGGAC CCCGCATCA TAGGTTTGA
TCTGGAGAAT AAGCTCTGA CACCAACCA ATGGAGAAA
GGGCACAGGG CTTGGCGTT GCCATAATC AGTACACAT
ACCAAAACAA GTGGTAAAG TCCTTAGAC AGCTGAAAG
AGGGAAGACA TTATGGACA CATCTCAAG CAAGACCAA
AGAGGGAGCG ACAAGTTGT ACTTACGCT TTAATACAT
TCACCAACCT GTGGTGCAG TCATTCGGA CATGGAGGC
TGAGGAAGTT TAGAGATGG AGACTTGTG CTGTTGAGG
AGGCCAGAGA GGTGACCAG TGGTTGCAG GCAACGGAT
GGGATAGGCT AAACGAATG CAGTCAGTG AGATGATTG
TGTTGTGAAA CAATTGATG TAGGTTTGC CATGCCCTC
AGGTTTTTGA TGACATGGG AAAGTTAGG AGGACACAC
AGGAGTGGAA CCCTCAACT GATGGAGCA CTGGGAAGA
AGTTCCGTTT GCTCCCATC CTTCAACAA CTTTACCTC
AAGGACGGGA GTCCATTGT GTCCCCTGT GCCACCAAG
ATGAACTGAT GGCCGAGCC GCGTCTCAC AGGGGCGGG
ATGGAGCATC GGGAGACTG TTGCCTAGC AAATCATAT
GCACAAATGT GCAGCTTCT TATTTCCAC GAAGGGACC
TCCGACTGAT GCCAACGCC TTTGTTCAT TGTGCCAGT
TGACTGGGTT CAACTGGGA AACCACCTG TCAATCCAT
GGAAAGGGAG ATGGATGAC ACTGAGGAC TGCTTGTGG
TGTGGAACAG GTGTGGATT AGGAGAACG CCACATGGA
GGACAAGACC CAGTCACGA ATGGACAGA ATTCCCTAT
TTGGGAAAAA GGAAGACTT TGGTGTGGA CTCTTATAG
GGCACAGACC CGCACTACT GGGCTGAGA CATTAAAGA
CACAGTCAAC TGGTGCGCA GATCATAGG GATGAAGAA
AAGTACATGG CTACCTATC ACTCAAGTT GCTACTTGG
GTGAAGAAGG TCCACACCT GAGTGTTA

An exemplary Spodweni virus lineage has the following nucleotide sequence (SEQ ID NO:13 which encodes SEQ ID NO:16; see Accession No. DQ859064, which is incorporated by reference herein:

atgaaaaacc caaaaagagc cggtagcagc cggcttgtca
atatgctaag acgcggtgca gcccgtgtca tccctccagg
aggagggctc aagaggctgc ctgtaggatt gctgttgggt
cggggtccga tcaaaatgat cctggccata ctggcattcc
tacgatttac agcaataaaa ccgtccactg gcctcatcaa
cagatgggga aaagtgggca aaaaagaggc catcaaaatc
ctcacaaaat tcaaggctga cgtgggcacc atgctgcgta
ccatcaacaa tcggaagaca aaaaagagag gagtcgaaac
tggaattgtg ttcctggcat tgctggtgtc tattgttgct
gtggaagtca caaaaaaggg ggacacctat tacatgtttg
cggacaagaa ggacgccgga aaggtggtga cctttgagac
tgaatctgga cccaaccgtt gctccatcca agcaatggac
attggacata tgtgtccagc tacaatgagc tatgaatgtc
ccgtgctgga accacagtat gagccagagg atgtcgactg
ttggtgcaac tcgacagcag catggattgt gtatggcaca
tgcacccaca agacaacggg agagacaaga cgttccagac
gttcaatcac cctgccatct catgcctcac aaaagttgga
gaccagatca tcgacgtggc ttgaatcccg cgaatactcc
aaatatctaa taaaggtgga aaactggatc ctccgcaatc
caggatatgc gttggtggct gcagtgattg gatggactct
gggcagcagt cgcagccaga agatcatctt tgtcactctg
ctcatgttgg tagcccccgc atacagcatc agatgcattg
gaattggaaa cagagacttc attgagggaa tgtccggtgg
cacctgggtg gacattgtcc tggaacatgg tggttgtgtg
acagtaatgt caaacgacaa acccacattg gactttgaac
tggtgacaac gaccgcaagt aacatggctg aggtcaggtc
ctactgctat gaagctaaca tatccgagat ggcatcggac
agcaggtgcc ccacacaggg ggaagcttat cttgacaaaa
tggccgactc ccagtttgtg tgcaagcgtg ggtacgttga
caggggctgg ggaaacggat gtggactctt tggaaaagga
agcattgtca cttgcgctaa gttcacgtgt gtgaaaaagc
tcacagggaa aagcattcaa ccggagaatc tcgagtaccg
ggtccttgtt tcggtgcacg cttcccaaca tggaggaatg
attaacaatg acaccaatca ccaacacgac aaggagaaca
gagcgcgcat tgatatcaca gctagcgctc cccgtgttga
ggtggaactt ggctcctttg gatccttctc gatggagtgt
gaaccccggt caggattgaa ctttggtgac ctgtattacc
tcaccatgaa caacaagcat tggctggtta atagagattg
gtttcacgat ctttccttgc catggcatac aggagccaca
tcaaacaatc atcactggaa caacaaggag gcgctggtag
aattcagaga agcccacgca aagaagcaga cggctgtggt
cctgggaagt caggaaggag ctgttcacgc agcactggcc
ggcgcactgg aggctgagtc tgatggacac aaagcgacta
tctactctgg acacttgaag cgtcgcttga agctagacaa
actgcgcctg aagggaatgt catatgcact ctgcacagga
gcattcacct tcgctcgcac cccctctgaa acaattcacg
gcaccgccac agtggagctg caatatgcag gtgaagatgg
gccgtgcaaa gttcccatag taattaccag tgacaccaat
agcatggcct cgacaggcag gctgatcaca gcgaatccgg
tggtcacgga aagtggagca aactcaaaga tgatggtcga
gattgaccct ccgtttggtg attcttacat tattgtgggc
actggcacaa caaaaattac ccaccattgg cacagagccg
gtagttcaat tggacgtgca tttgaggcta ccatgagagg
agcaaaacgg atggcggtcc tcggcgacac cgcttgggac
tttggctctg ttgggggcat gttcaactcc gttggaaagt
ttgtccacca ggtgtttgga tcagcattta aggcattgtt
tggaggcatg tcctggttca cacagctcct gataggattt
ctgctcatat ggatgggttt gaacgcacgc ggtggaaccg
tggccatgag cttcatgggc attggggcta tgctgatttt
cccagccacc tcggtgtcag gagacacagg atgctcggtt
gacatatcca gaagggaaat gcggtgcggg agcggcatat
tcgtgtacaa tgacgttgac gcatggcgaa gccgctacaa
ataccatcct gaaaccccca gagctttggc cgctgccgtg
aaaacggctt gggaagaagg gacctgtggc attacctcag
tgagcagaat ggaaaacctg atgtggagct ctgtggctgg
agagttgaat gcaatccttg aggacaattc agtgccattg
acagtcgtcg ttggcgagcc aaaatatcca ctgtacaatg
ctccaaagag gctgaaacca ccagcatcag agttaccgca
ggggtggaag tcctggggaa agtcatactt tgtctcagcc
gcaaaaaaca acaactcctt tgtggtagat ggtgacacca
tgaaggaatg cccaagacag aagcgagcat ggaacagctt
gagaatagag gatcatgggt tcggagtctt ccacactagc
atctggctga aattccatga ggacaactcc accgaatgtg
acacagctat cataggaacg gcggttcgcg ggaaggaagc
cgttcatagt gacttgggct actggataga gagtgagcgc
aatgacacat ggaggctctc tcgagcgcac ctgatcgaag
caaagacatg tgaatggcca cggtcgcaca cactgtggac
ggacggagtg gaagagagcg agctgatcat tccacgtggc
ttagccggtc ctttcagcca tcataacacg cgtgctggct
acaagactca gaataaaggt ccctggcatt taggtgatgt
tgaaattcag ttcgccacgt gccccggaac aaccgtggtc
caggaccaag agtgcaggga caggggcgct tctctacgca
cgaccacagc tagtggaagg gtaatcaatg aatggtgctg
caggtcgtgc accatgcctc cactcagttt caagacaaaa
gatggatgtt ggtatgcaat ggagatacgt cctgtgaaag
aacaagagtc aaacctcgtg cgatcgcacg tcactgccgg
aagcacagac cacatggacc atttctctct cggattagta
gtggtcatgt tgatggtgca agaaggtatg aagaagagaa
tgacatcaaa agcaataatc acctcagcgg cctttctcct
ggcggttatg atagtgggag gtttcacgta ccaggatttt
gggaggctgg tggtattggt gggtgctgca tttgctgaga
tgaacactgg aggtgacgtt gcgcacctgg cgctggtggc
agcgtttaaa gtgaggccag cgatgctggt ctcattcatg
ttcagagcct tgtggacccc cagggagtca ctgcttttag
ctctggctgc ctgcctcctg caggtgtcag tgacaccact
ggatcattcc atcatgatcg tggttgatgg gattgcgctg
tcctggttgt gtctgaaagc catcttggtg ccgcgtaccc
caaacatagc ccttcctctt ctcgctatgc tgtcacccat
gctccaaggt accaccattg tggcatggcg agctatgatg
gcggccctgg ctgtcataac cttggcttcc atgaagcatg
gaaggggtgt aaaaaagacg tttccctaca ccatcggatg
catccttggc agcatgggct tagttgaaaa cttggggttg
gttggcctcc tcttgttgac agcctcaaaa aagaggagtt
ggcctccgag tgaggtgatg acggctgtcg gactgatctg
tgcaattgtg ggcggactaa ccaagaccga cattgacatg
gcgggaccca tggcagccat aggactgctg gtggtgagct
atgtggtttc tggcaagagt gtggacatgt acattgaaaa
ggtgtgtgac atatcatggg acaaggacgc tgaaataaca
ggcacaagtc cgcggctgga tgtggctctc gacgacagtg
gagatttctc acttatccag gatgacgggc cccccactcg
agagattgtg ttgaaggtgt ttctgatgtg tgtttgcggt
gtcagcccca tagccatccc ctttgcagcc gctgcttggt
tcgtgtacat taaatcaggg aaaagaagcg gcgccatgtg
ggacattcca tccccaagag aagtgaaaaa aggggaaaca
acggctggag tgtacagaat catgacgcgt aaattgctgg
gcagcacaca ggtgggagcc ggagtaatgc atgaaggtgt
ttttcacaca atgtggcacg tcacaaaagg ttcggccctt
cggagtggtg agggacgcct agatccatac tggggaaacg
tgaagcagga tttgatctct tactgcggac catggaaacc
ggatgggaaa tgggacggcg tgtcggaagt ccaactgata
gcggtcgccc caggtgagcg cgccagaaat gtgcagacaa
aaccaggagt gttcaagacc actgatgggg aaatcggggc
cttggccctt gacttcccag gcggaagttc aggctccccg
ataattgaca aaaatggaca tgtaattggc ctgtatggaa
atggtgtcgt ggtcaggagt ggaagctacg tgagtgccat
catgcagaca gagaagatgg aggaacccgc agttgactgc
tttgaggagg acatgctgag aaaaaagaag ctgacggtgc
tcgacctcca tccaggagct ggaaaaactc gaagagtgct
ccctcagatc gtcaaggctg caattaagaa acgcctacgc
acggtaatcc tggcacccac ccgagtggtg gcagctgaga
tggctgaggc actaaaagac cttccaataa ggtacatgac
tccggcagtt tcagccaccc atgatggcaa tgagattgtt
gaccttatgt gccacgccac ttttacatca aggctaatgc
aaccaattag ggtgcctaat tacaatctat atataatgga
tgaggcccac ttcacagatc ctgcaagcat cgctgcaaga
gggtacatag caacaagagt ggacatggga gacgccgcgg
ccatcttcat gacggccacc cctcctggca gcactgaagc
tttcccggat tcaaacgccc ccatcacaga tgttgaaaca
gaggttcctg acaaggcgtg gaattctgga tttgaatgga
tcactgatta cccagggaaa accgtttggt ttgtccctag
tgtcagaatg ggcaatgaga tctcggcctg cctcacaaaa
gccggcaaat cggttatcca actcagccgg aaaacctttg
aaacagagta ccagaagaca aagaatggtg agtgggactt
tgtcgtgacc actgacatct cagaaatggg agccaacttc
aaggccgaca gagtcataga ctcacggaaa tgcttgaagc
cagtgattct ggatgacatg gaagagagag ttgttcttgc
cgggccgatg gcagtaacac catccagcgc agctcaacgc
agaggaagaa ttggaagaaa ccccaacaaa actggagatg
agttctatta cggggggggc tgtgccgcaa cggatgatga
ccatgctcat tgggtagagg ctagcatgct gcttgacaac
atctacctcc aggacaacct cgttgcatct ctgtacaagc
cagaacaagg aaaggtctcg gcaatagaag gggagttcaa
actgagagga gaacagagga aaaccttcgt ggagctgatg
aagagagggg acttgccagt gtggttgtca tatcaagtgg
cggcctccgg actcagctat actgaccggc gctggtgctt
tgatggaaaa aacaacaaca ccatcctgga ggactgcgtc
cccgtcgagg tgtggacaaa atttggagag aaaaagattc
tgaagcccag atggatggac gctcggatct gctctgatca
tgcctctttg aagtctttca aggagtttgc tgcaggaaag
agaacaatag ccactggctt aattgaggct tttgggatgc
ttcccgggca catgactgag agattccagg aggccgtcga
caatttggcc gtgttgatga gggccgaggc aggctctagg
gcacacagaa tggctgcagc acagctccct gagacaatgg
aaaccatcct gctcctcagc ctgctggcat tcgtgtcact
tggtgtattt tttgtactga tgagggcaaa agggttagga
aaaatggggt ccggcatgat cgtgctggca ggaagtggct
ggctcatgtg gatgtctgag gtggaaccag cccgcatagc
ttgtgtggtg atcatagtgt ttctgctaat ggtcgttctg
attccggaac cggagaagca gcgctctccc caggacaatc
agctggctct aattatcttg atcgcgacgg gcctcatcac
gctcatcgcg gccaatgagc tgggttggtt agaaagaaca
aagagtgacc tcaccaggcc gttttggaga gaacacgctg
agccaacagg agggagaggg ttttccttct cgctggacat
tgacctgcgg ccggcatcgg cctgggcaat atatgccgct
atgacaaccc tgatcacacc gacagtccaa cacgctgtga
ccacatcgta caacaactac tctctcatgg ctatggccac
tcaggccgga gttctttttg gcatgggacg gggggtgcct
ttttacaaat gggactttgg cgtgccactc ettatgetgg
gctgctactc acaacttacc ccactcaccc tgatcgtggc
tctcgtgatg ctagccgctc actatctcta tctcatcccc
gggctccagg caacggccgc cagggccgcc caacgaagga
cggctgctgg aataatgaaa aacccagcgg tggatggaat
tgtggtaact gacatagacc caatccaaat cgatccaaat
gtcgaaaaga agatgggcca ggtcatgctc atctttgtgg
etttggegag cgcggttctc atgagaacgg catggggttg
gggagaggct ggtgcccttg catcggcagc agctgccacc
ctatgggaag gggctcccaa caagtactgg aattcatcaa
cggctacatc cttgtgcaac atatttcggg gaagttatct
ggcaggtccc tccctcatct acaccgtcac acgcaatgca
ggtatcatga agaaaagggg cggtggaaat ggagaaaegg
tgggcgagaa atggaaggag cgcttgaatc ggatgaccgc
gcttgaattc tacgcctaca agcggtcagg aataactgaa
gtgtgcagag aacccgccag aagagccttg aaggatggag
tcgtcacagg aggacacgct gtctcccgcg gaagcgcaaa
gctgcgatgg atggtggaac gtggccacgt caatctagtg
ggacgcgttg tcgacctcgg atgtggaagg ggtggctgga
gttactacgc cgcatctcaa aagcaagtcc tcgaggtgag
aggctacaca aaagggggag cgggccacga ggagcccatg
aatgtccaaa gttatggttg gaacatagtg cgactcaaga
gtggagtgga cgttttttat ctaccatcag aaccatgtga
cacgctgctc tgtgacattg gagagtcatc ctcgagccca
gcagtggaag aagcccggac tctgagagtg ctcgggatgg
ttgaaacctg getggaaegg ggcgtaaaga acttctgcat
caaagtgctc tgcccgtaca ccagtgccat gattgagcgg
ctggaagccc tccagcgteg ctacggagga ggcctggtga
gggttccact ctccagaaat tccacccacg aaatgtactg
ggtctctgga gcaaaatcaa acatcatcag gagtgtgaat
gccaccagcc agctgctcat gcacagaatg gacatcccca
cgcggaaaac aaagtttgaa gaagacgtca atctggggac
cggaaccagg gcagttgaaa gcagagctga ccctcccgac
atgaaaaaac taggcagccg gattgagcgg ttgagaaagg
aatatggatc cacttggcac cacgatgaaa accaccccta
caggacatgg cattaccacg gcagttatga ggctgacacg
caaggctccg cctcctcaat ggtcaacggc gtggtgcgtc
tcctctcaaa accatgggat gcattgagct cggtcaccaa
cattgctatg acggacacaa ctccgtttgg acagcagcgg
gtgttcaagg agaaagtgga cacccggact ccagacccca
agcagggcac gcaaagagtc atggccataa catcacaatg
gctgtgggac cgcctagcaa gaaacaagac ccctcggatg
tgcacgcgac aggaattcat aaacaaggtc aacagtcacg
cggcgttggg acccgttttt agagaacagc agggatgggg
ttcagcggcc gaagcggtgg tagatcctag gttttgggag
ctcgttgaca atgaaagaga agcccatttg agaggggagt
gcttgacctg tgtctacaac atgatgggga aaagagaaaa
gaagctcggt gaattcggga aggcaaaagg cagcagagcc
atttggtaca tgtggctggg agcccgcttc ctcgagttcg
aggccctggg cttcctcaat gaagaccact ggttaagcag
agagaactct ggagggggag ttgagggctt gggcctccaa
aaacttggat acatccttga agagatcagc aggaggccag
gaggcaaaat gtatgccgat gacacggctg gctgggacac
ccgcatcacg aaatgcgacc tagaaaatga ggcgcgcatt
ttggaaaaaa tggacgggat ccacaaaaaa ctcgcacggg
ccgtcatcga gttgacatac aagcataagg ttgtgagagt
cttgagacca gcaccacaag ggaaggtcgt tatggacatc
atctccaggc cagaccaaag ggggagtggg caggtggtta
cttatgccct caacacctat acaaacttgg tggtgcagct
gatccgtaac atggaagcag aggctgtcat caatgaaaga
gacatggagg agctccaaaa cccatggaaa gtcatcaatt
ggctagaagg aaatggatgg gacagactcc gctcgatggc
agtgagtgga gatgactgtg tcgtgaaacc aatggatgat
aggttcgcct atgcactgaa tttcctcaat gacatgggca
aggtcagaaa agatgtccag gaatggaagc cctcgccggg
gtggacaaac tgggaagaag tgcccttttg ctcccaccac
ttcaacaagc tcccgatgaa ggatggaaga acaataatag
ttccctgccg gcaccaagat gagttgatag gcagggctag
agtttctcca ggaaaaggct ggtcactcag tgaaacagca
tgcttgggca agtcttatgc ccagatgtgg ctactgttgt
actttcacag gagagatctc cgactcatgg caaacgcaat
ctgctctgct gtaccggtga gttgggtgcc cacggggaga
acaacctggt ccatccatgg gcgtggagag tggatgacaa
cagaggacat gctagaggta tggaacagag tgtggatcat
agagaatgag tacatggagg acaagacccc tgtcacagag
tggaccgatg ttccacactt gggaaagaga gaagacttgt
ggtgcggctc ccttattgga cacaggccaa gaagcacatg
ggcagagaac atctgggctg ccatttatca agtgcgccga
gcaatcggcg aaactgaaga atatagagac tacatgagca
cacaggtccg ctatggctcg gaggaagggc caagcgctgg
tgtgttgtaa

### EXAMPLE 3

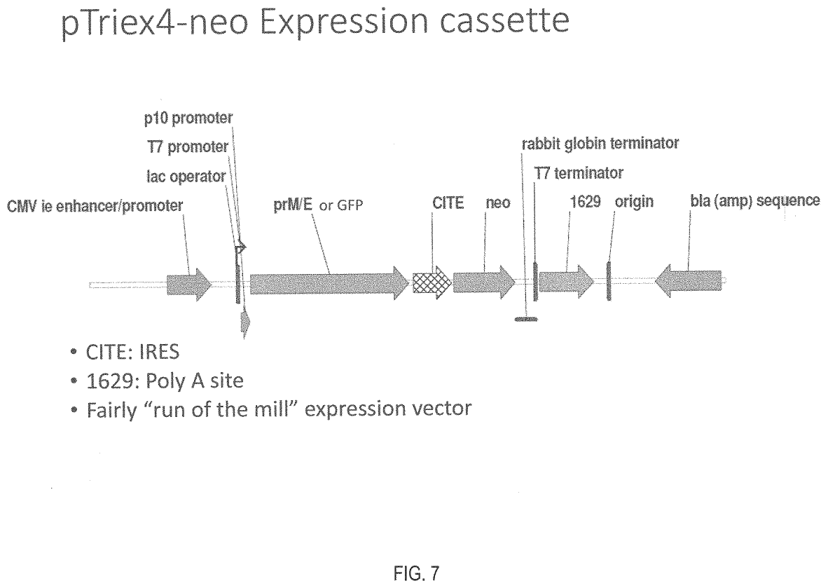

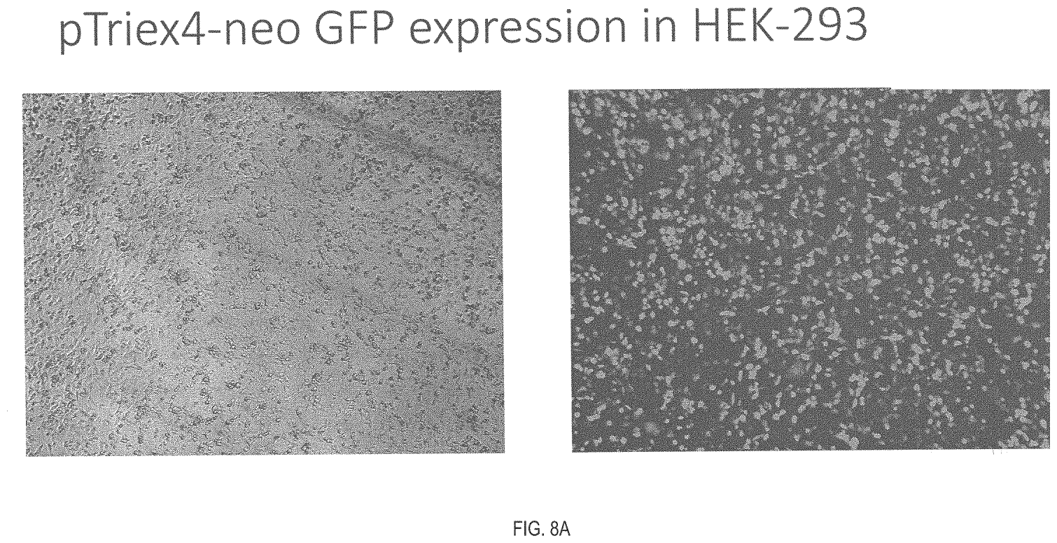

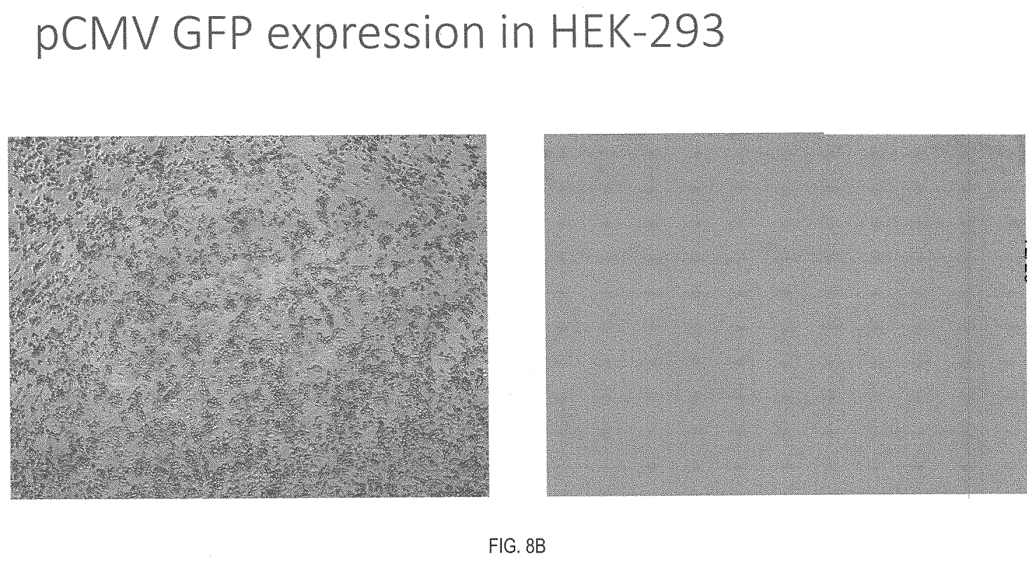

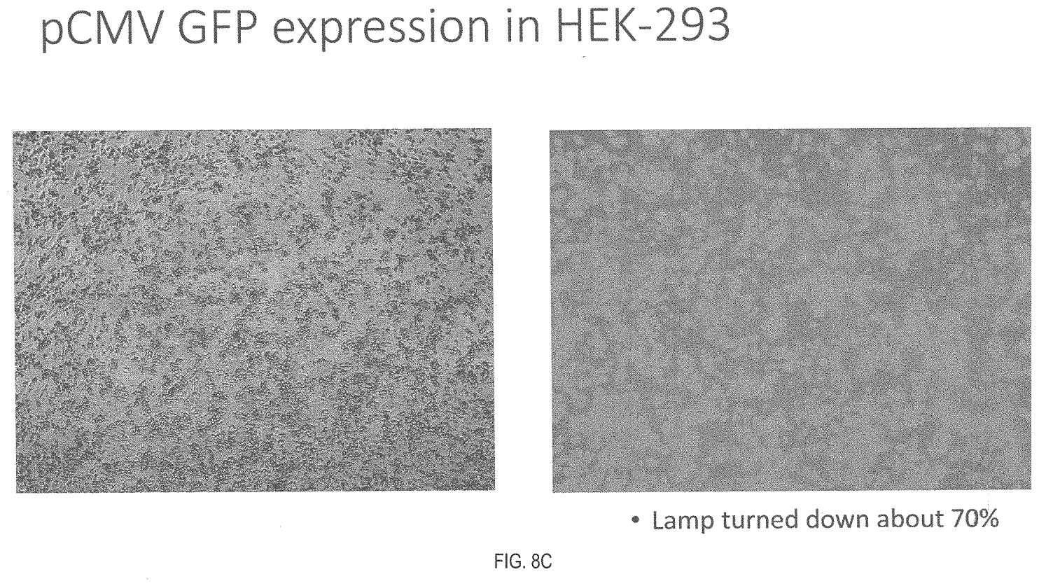

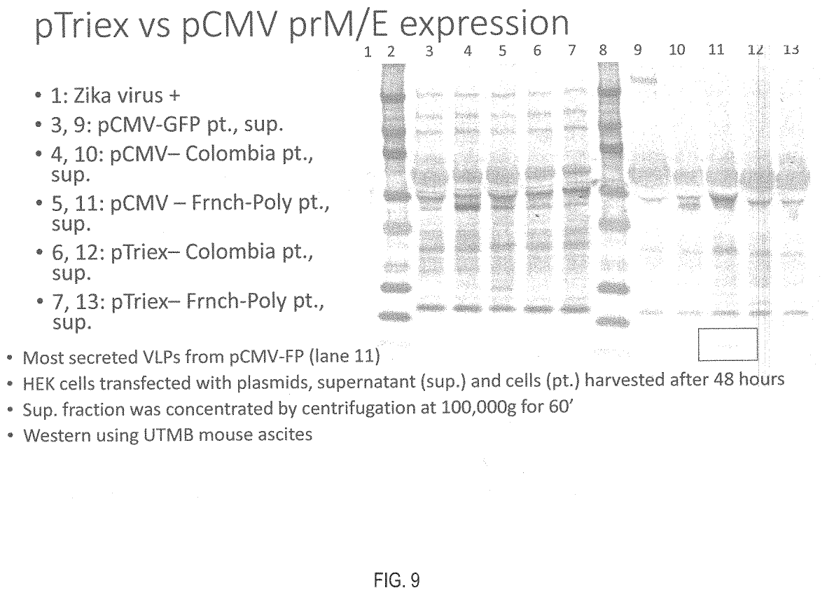

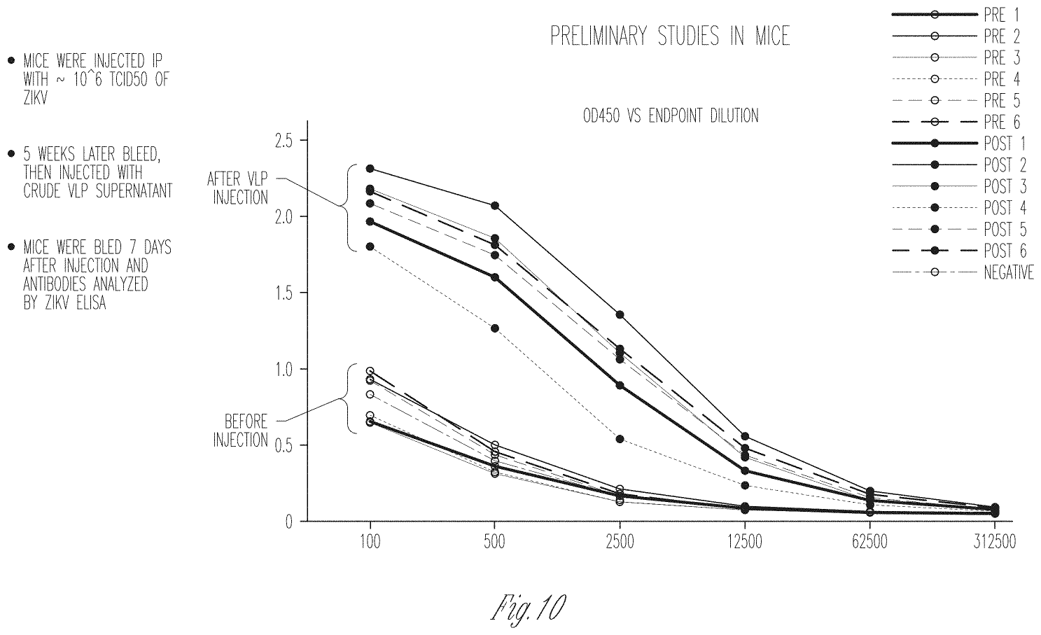

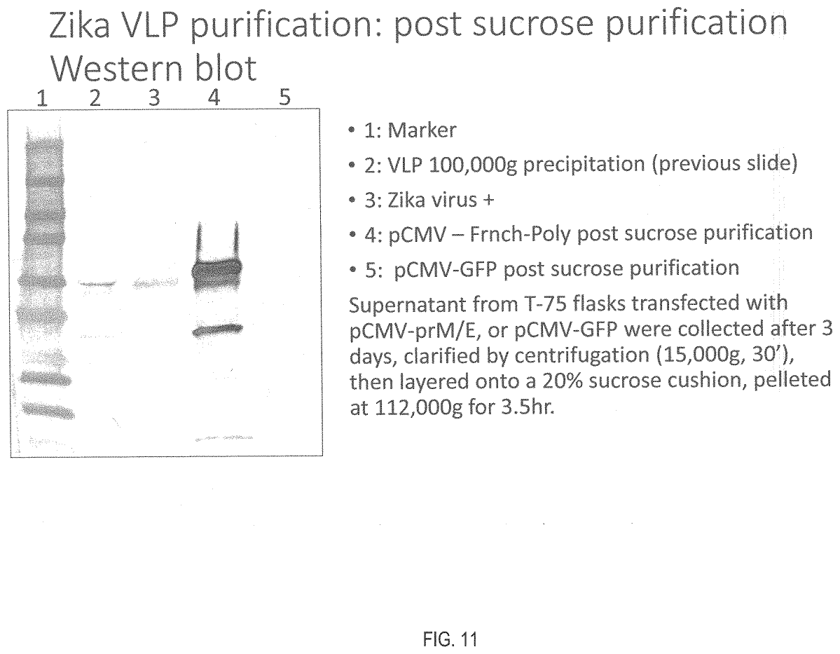

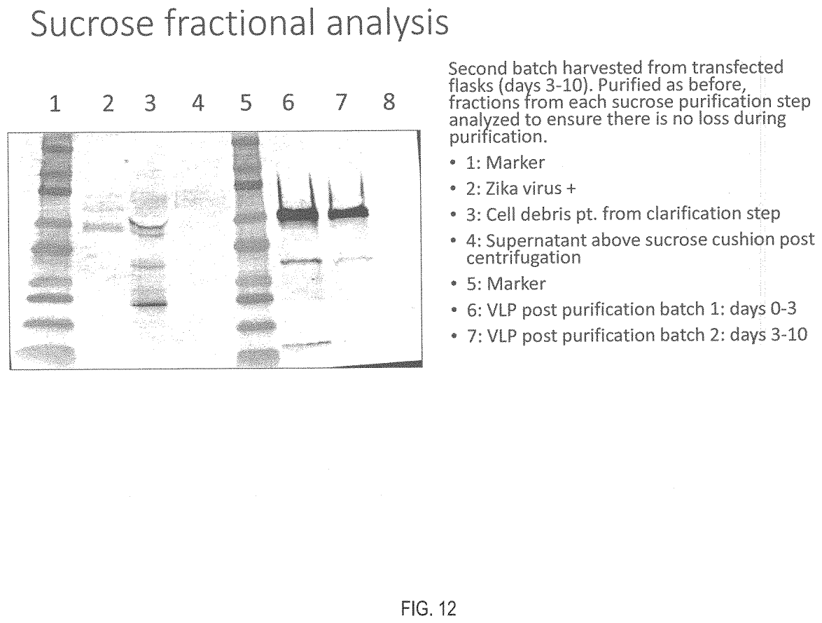

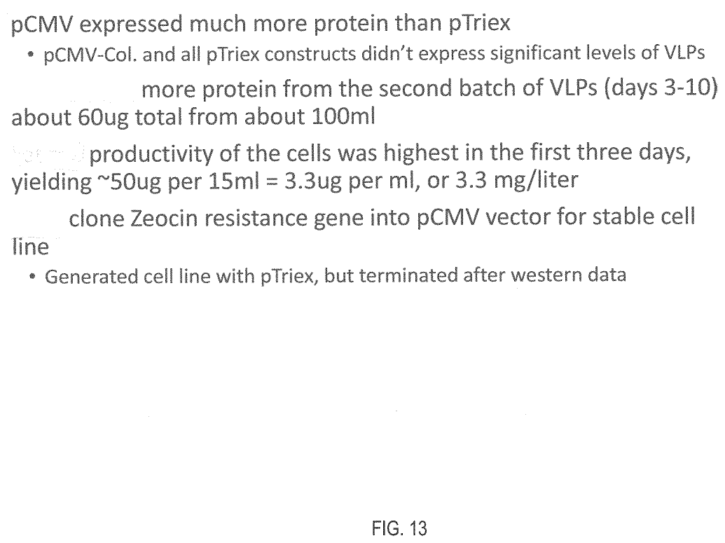

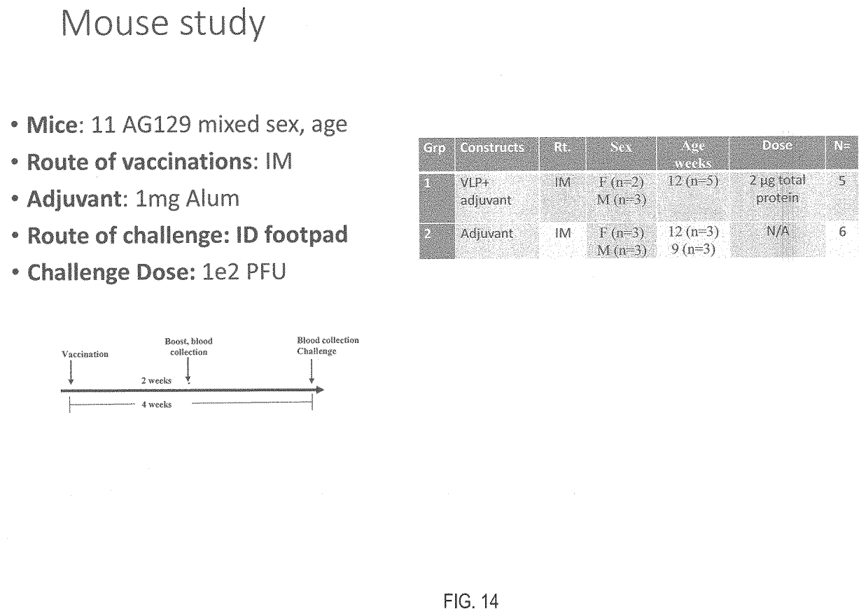

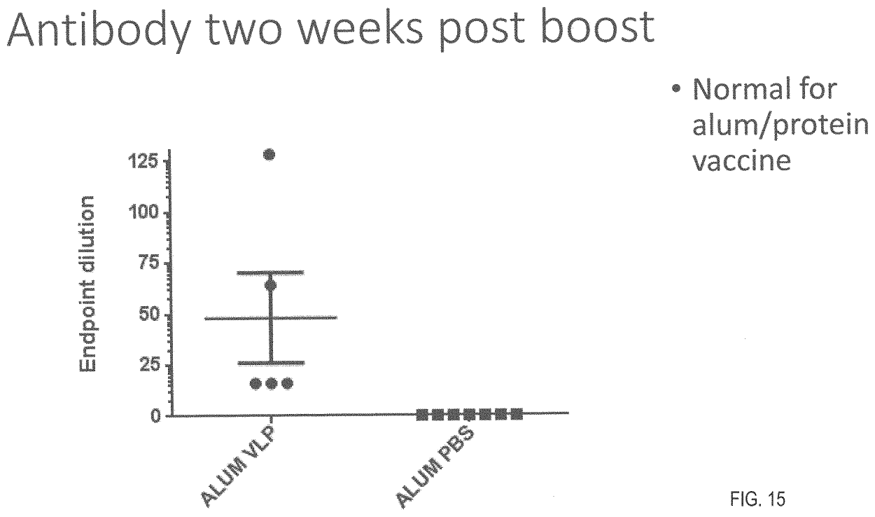

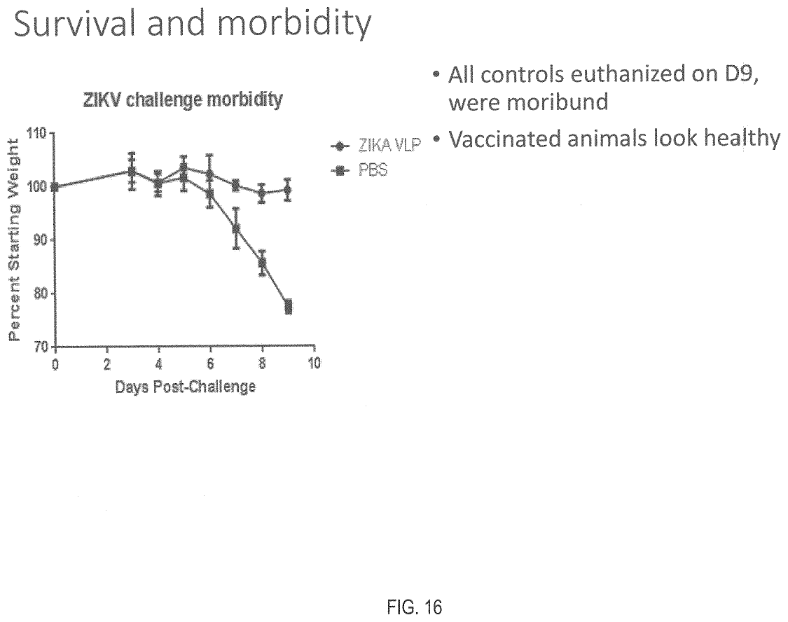

Exemplary vectors expressing CFP were transfected into HEK293 cells and expression was assessed (FIGS. 7-8). prM/E sequences were also expressed from the two vectors in HEK cells and supernatants and cells analyzed 48 hours later (FIG. 9). Supernatants were concentrated by centrifugation at 100,000 g for 60 minutes. Western blots were analyzed using University of Texas Medical Branch (UTMB) mouse ascites. More VLPs were secreted from pCMV-FP transfected cells (lane 11 in FIG. 9) than pTriex transfected cells (lane 13). Sucrose purified fractions were subjected to Western blot (FIGS. 10-11). pCMV-prM/E SC purified pellet (pt) appeared to contain high levels of E protein while pCMV-GFP pt did not, indicating that staining was specific to expression of prM and E genes. In summary, a pCMVvector expressed more protein than a pTriex vector. VLPs collected at days 3-10 provided for about 60 μg total protein from about 100 mL. On day 3 the productivity of the cells was about 50 μg per 15 mL (3.3 μg per mL, or 3.3 mg/L). For stably transfected cells, a marker, e.g., a Zeocin resistance gene, may be introduced into the vector that expresses prM/E.

ZIKV VLPS (ZIKVLPs) formulated with alum were injected into 6-8-week-old interferon deficient A129 and AG129 mice. Control mice received PBS/alum. Animals were challenged with 200 PFU (>400 LD₅₀s) of ZIKV strain H/PF/2013. All vaccinated mice survived with no morbidity or weight loss while control animals either died at 9 days post challenge (AG129) or had increased viremia (A129). Neutralizing antibodies were observed in all ZIKVLP vaccinated mice.

### EXAMPLE 4

### Materials and Methods

### Cells and Viruses

African Green Monkey kidney cells (Vero) and Human embryonic kidney 293 (HEK293) were obtained from ATCC (ATCC; Manassas, Va. USA) and grown in Dulbecco's modified Eagle medium (DMEM) supplemented with 10% fetal bovine serum (FBS; Hyclone, Logan, Utah), 2 mM L-glutamine, 1.5 g/l sodium bicarbonate, 100 U/ml of penicillin, 100 μg/ml of streptomycin, and incubated at 37° C. in 5% CO2. ZIKV strain H/PF/2013 (GenBank:KJ776791), was obtained from Xavier de Lamballerie (European Virus Archive, Marseille France). Virus stocks were prepared by inoculation onto a confluent monolayer of Vero cells.

### Animals

Mice of the 129/Sv background deficient in alphalbeta interferon alpha/beta/gamma (IFN-α/β/IFN-γ) receptors (AG129 mice) were obtained from B&K Universal Limited (Hull, England) and were bred in the pathogen-free animal facilities of the University of Wisconsin-Madison School of Veterinary Medicine. 5-week-old BALB/c mice (Tire Jackson Laboratory, Maine, USA) were used for wild-type vaccination studies. Groups of mixed sex mice were used for all experiments.

### Production and Purification of ZIKV VLPs

The prM and E genes of ZIKV strain H/PF/2013 with nascent signal sequence were cloned into a pCMV expression vector under the control of a cytomegalovirus (CMV) promoter and CMV polyadenylation signal (pCMV-prM/E, FIG. 1), Endotoxin free, transfection grade DNA was prepared using Maxiprep kit (Zymo Research, Irvine, Calif.). VLPs were expressed by transfecting 90% confluent monolayers of HEK293 cells in a T-75 flasks with 15 μg of pCMV-prM/E using Eugene HD (Promega, Madison, Wis.) transfection reagent according to manufacturer protocol. The 10 ml supernatant was harvested 72. hr after transfection, and clarified by centrifugation at 15,000 RCF for 30 min at 4° C. Clarified supernatants were layered onto a 20% sucrose cushion and ultra-centrifuged in a SW-28 rotor at 112,000 RCF for 3.5 hours at 4° C. Pellet (PT) and supernatant (SUP.) fractions at each step were saved for analysis by SDS-PAGE and Western blot, Post sucrose cushion PT were resuspended in Phosphate Buffered Saline (PBS) pH 7.2. Total protein in VLP preparations was quantified by Bradford assay. VLP specific protein was determined by comparing Zika specific bands on SDS-PAGE gels to known concentrations of BSA using IntageJ software.

### Western Blot

VLP fractions were boiled in Laemmli sample buffer (BioRad, Hercules, Calif., USA) and resolved. on a 4-20% SDS-PAGE gel (Biorad) by electrophoresis using a Mini-PROTEAN 3 system (BIO-RAD, Calif.). Gels were electroblotted onto nitrocellulose membranes using a Turboblot® system. Membranes were blocked in 5% (W/V) skim milk and probed with mouse hyper immune ascites fluid primary antibody (1:5000) and goat anti-mouse HRP conjugated secondary antibody (1:5000). Membranes were developed using a solid phase 3,3′,5,5′-tetramethylbenzidine (TMB) substrate system.

### Transmission Electron Microscopy

Samples were negatively stained for electron microscopy using the drop method. A drop of sample was placed on a Pioloform™ (Ted Pella, Inc.) carbon-coated 300 Mesh Cu grid, allowed to adsorb for 30 seconds, and the excess removed with filter paper. Next, a drop of methylamine tungstate or uranyl acetate (Nano-W, Nanoprobes Inc.) was placed on the still wet grid, and the excess removed. The negatively stained sample was allowed to dry, and was documented in a Philips CM120 (Eindhoven, The Netherlands) transmission electron microscope at 80 kV. Images were obtained using a SIS MegaView Ill digital camera (Soft Imaging Systems, Lakewood. Colo.).

### Vaccination and Viral Challenge

Each of the following animal studies was performed as one biological replicate. For VLP formulations, the indicated dose of sucrose cushion purified 2.5 VLPs was mixed with 0.2% Inject Alum (Thermo Scientific) according to manufacturer's protocol. Groups of AG129 mice were injected intramuscularly (IM) with VLPs mixed with alum (n=5) or PBS mixed with alum (n=6) at 6 weeks of age, and again at 8 weeks of age. Sub-mandibular blood draws were performed pre boost and pre challenge to collect serum for analysis by neutralization assays and for passive transfer studies. AG129 mice were challenged with 200 PFU of ZIKV strain H/PF/2013 in 25 volumes by intradermal (ID) injection into the right hind footpad at 11 weeks of age. Barbie mice were vaccinated once at 5 weeks of age as above, and challenged at 13 weeks of age with 200 PFU of H/PF/2013 in 50 μL by retro orbital injection (IV route).

Following infection, mice were monitored daily for the duration of the study. Mice that were moribund or that lost greater than 20% of starting weight were humanely euthanized. Sub-mandibular blood draws were performed on day two post challenge (PC) and serum collected to measure viremia.

Eight week old AG129 mice were used for passive transfer studies Five naive mice were injected intraperitoneally (IP) with 500 μL of pooled serum from VLP vaccinated, diluted serum (1:5 n=4, 1:10, n=4), or serum from PBS/alum (n=5) treated mice. At 12 h post transfer, mice were challenged with 20 PFU in 25 μl as above.

### Viremia Assays

Viremia was determined by TCID₅₀ assay. Briefly, serum was serially diluted ten-fold in microtiter plates and added to duplicate wells of Vero cells in 96-well plates, incubated at 37° C. for 5 days, then fixed and stained with 10% (W/V) crystal violet in 10% (V/V) formalin. Plates were observed under a light microscope to determine the 50% tissue culture infective doses (TCID₅₀s). Serum samples were also tested for viral RNA copies by qRT-PCR. RNA was extracted from 0.02 ml of serum using the ZR Viral RNA Kit (Zymo Research, Irvine, Calif.). Viral RNA was quantified by qRT-PCR using the primers and probe designed by Lanciotti et. al (Lanciotti et al., 2008). The qRT-PCR was performed using the iTaq Universal Probes One-Step Kit (BioRad, Hercules, Calif.) on an iCycler instrument (BioRad, Hercules, Calif.). Primers and probe were used at final concentrations of 500 nM and 250 nM respectively. Cycling conditions were as follows: 50° C. for 10 min and 95° C. for 2 min, followed by 40 cycles of 95° C. for 15 sec and 60° C. for 30 sec. Virus concentration was determined by interpolation onto an internal standard curve made up of a 5-point dilution series of in vitro transcribed RNA, with the lowest copies per reaction being 100.

### Neutralization Assay

Serum antibody titers were determined by microneutralization assay. Briefly, serum was incubated at 56° C. for 30 min to inactivate complement and then serially diluted two-fold in microtiter plates. 200 PFUs of virus were added to each well and incubated at 37° C. for 1 h. The virus-serum mixture was added to duplicate wells of Vero cells in 96-well plates, incubated at 37° C. for 5 days, then fixed and stained with 10% (W/V) crystal violet in 10% (V/V) formalin, then observed under a light microscope. The titer was determined as the serum dilution resulting in the complete neutralization of the virus.

### Plaque Reduction Neutralization Test

Serum samples were serially diluted, mixed with 200 PFU of the ZIKV H/PF/2013 strain and incubated for 1 hr at 37° C. This serum/virus mixture was added to confluent layers of Vero cells in 96 well plates and incubated for 1 hr at 37° C., after which the serum/virus mixture was removed and overlay solution (3% CMC, 1× DMEM, 2% FBS and 1× Anti/Anti) was added. After 48 hrs of infection, the monolayers were fixed with 4% PFA, washed twice with PBS, and then incubated with ZIKV hyperimmune mouse ascitic fluid (1:2000, UTMB) diluted in blocking solution (1× PBS, 0.01% Tween-20 and 5% Milk) and incubated overnight at 4° C. Plates were washed three times with PBS-T and then peroxidase-labeled goat anti-mouse secondary antibody (1:2000) was incubated on monolayers for 2 hours at 37° C. Following incubation, cells were washed a final three times with PBS-T and developed using 3-amino-9-ethylcarbazole (AEC)-peroxidase substrate. The amount of formed foci were counted using an ELISPOT plate reader (ImmunoSPOT-Cellular Technology); quality control was performed to each scanned well to ensure accurate counting. Neutralization percentages (Nx) were calculated per sample/replicate/dilution as follows:

$$Nx \{ 100 - [ 100 ( \frac{A}{Control} )$$

Where A corresponds to the amount of foci counted in the sample and Control is the geometric mean of foci counted from wells treated with cells and virus only. Data of corresponding transformed dilutions (Log(1/Dilution)) against neutralization percentages per sample was plotted and fitted to a sigmoidal dose-response curve to interpolate PRNT₅₀ and PRNT₉₀ values (GraphPad Prism software).

### Results

### Expression and Purification of Soluble, Zika VLPs

To generate Zika VLPs (ZIKVLPs), we cloned the prM/E genes with native signal sequence into a pCMV expression vector (pCMV-prM/E) (FIG. 1A), transfected HEK293 cells and harvested supernatants (supe.) 3 days post transfection. 78 μg total protein was recovered from post sucrose purification of which 21.6 μg was ZIKVLP protein. Western blot analysis of this pCMV-prM/E supe. revealed expression of an about 50 kDa size band (FIG. 1B, lane 2) that corresponded in size to the predicted size of the Zika virus E gene, and additionally matched positive control Zika virus stocks (FIG. 1B, lane 3). To test the hypothesis that expression of Zika prM and E genes spontaneously form extracellular particles, supernatants from pCMV-prM/E and pCMV-GFP (negative control) transfected cells were centrifuged on a sucrose cushion (SC) sufficient for pelleting of flavi virus particles from cell culture proteins (Merino-Ramos et al., 2014). pCMV-prM/E SC purified pellet (pt.) appeared to contain high levels of protein, indicating that staining was specific to expression of prM and E genes. To determine if the immune reactive extracellular particles were virus like in nature, we performed transmission electron microscopy (TEM) on pCMV-prMiE SC pt. material. TEM revealed virus like particles with a size that ranged from 30-60 nm, and a typical size of about 50 nm (FIGS. 1C-E).

### Administration of ZIKVLPs is Immunogenic and Protects Highly ZIKV Susceptible α/β/γ Interferon Deficient (AG129) Mice

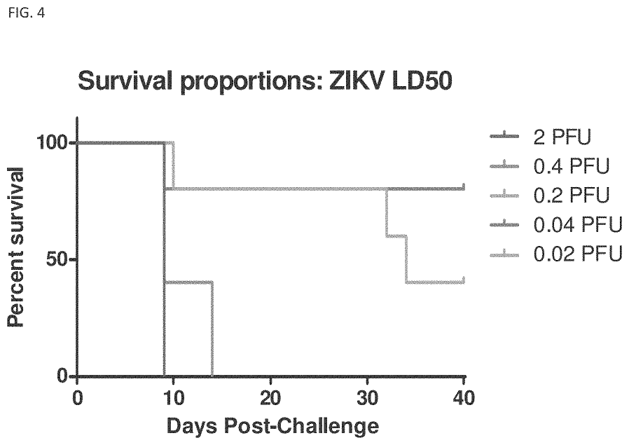

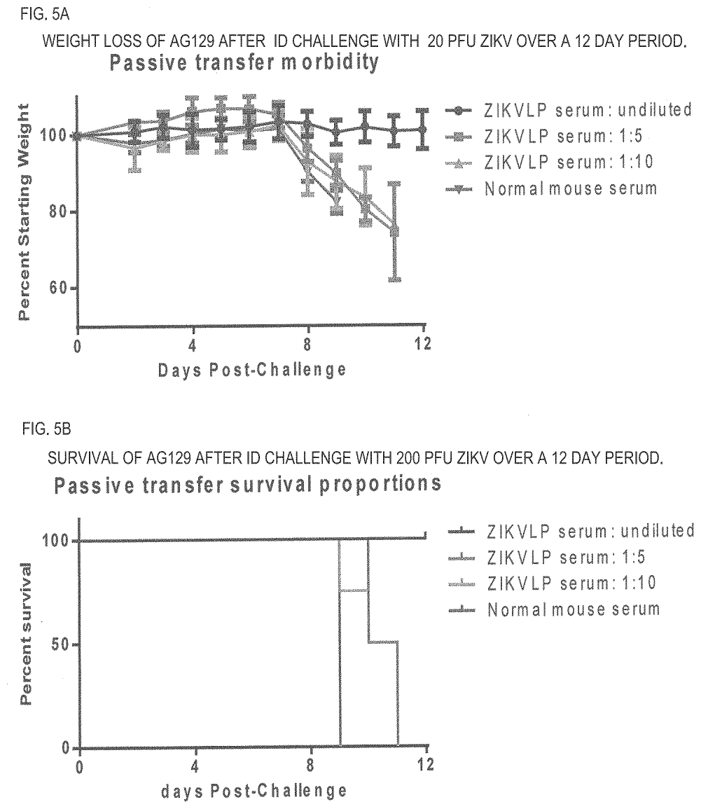

First, the LD₅₀ of the H/PF/2013 strain in 12 week-old mixed sex AG129 mice was determined. Groups of mice (n=5) were infected with 5-fold serial dilutions from 2 PFU to 0.02PFU of ZIKV and monitored for 4 weeks following the last mortality. All mice infected with 2 or 0.4 PFU died within the first week of challenge (FIG. 4), while lower doses killed only 1 to 2 mice within the first two weeks. Interestingly, 2 mice infected with 0.2 PFU ZIKV became ill and were eutlianized due to weight loss and paralysis 4.5 weeks following challenge. The resultant LD₅₀ value in PFUs was calculated to be 0.19 PFU by the Reed-Muench (REED and MUENCH, 1938) method.

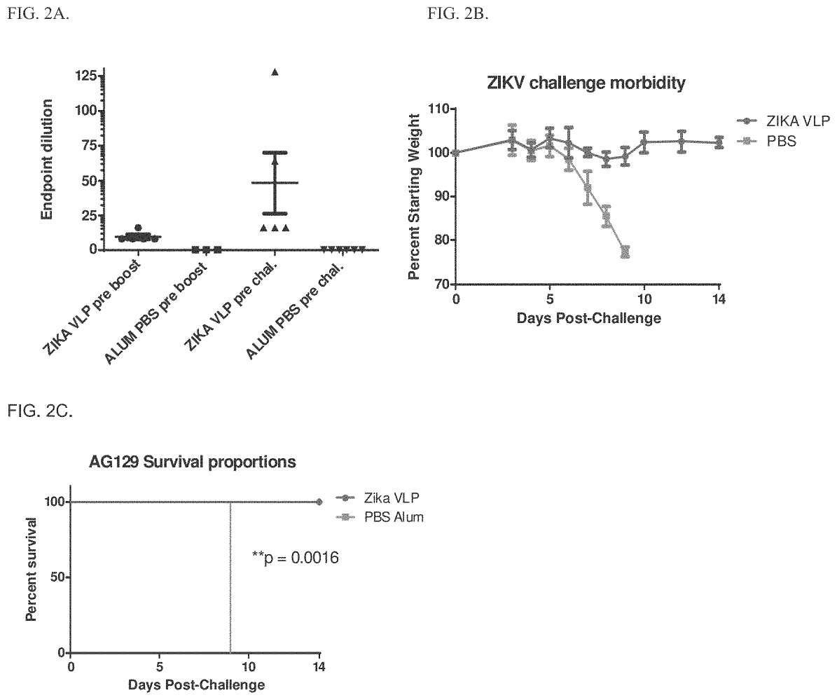

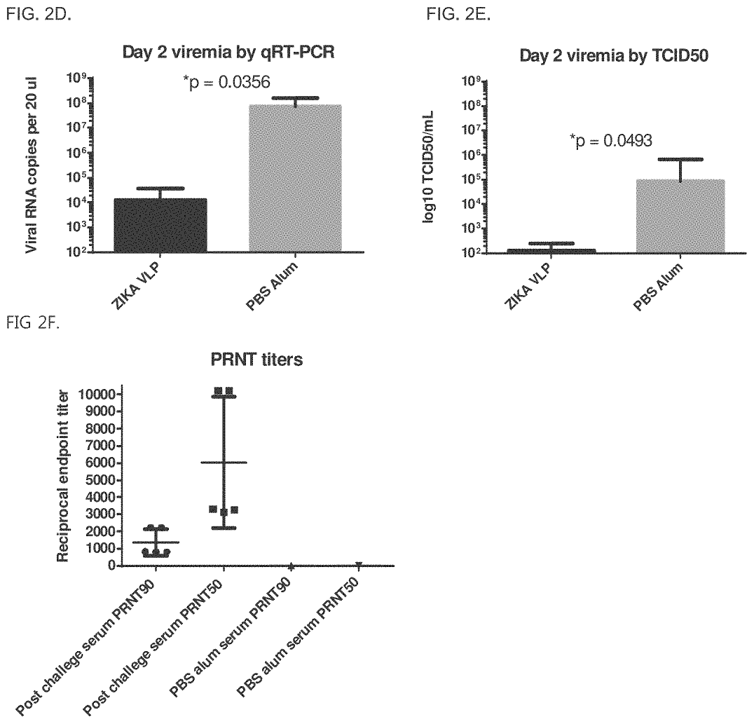

To determine if ZIKVLPs are immunogenic and protective in highly susceptible AG129 mice, groups of mice received a prime and boost of 450 ng ZIKVLPs. AG129 mice that received ZIKVLPs developed low levels (GMT=1:9.2) of neutralizing antibodies (nAbs) at two weeks post administration (FIG. 2A), that increased two weeks after boost (GMT=1:32). Five weeks after primary vaccination, all mice were challenged with 200 PFU (>1000 LD₅₀s) of ZIKV by the ID route. Mice administered. ZIKVLPs maintained weight, while mice that received PBS/alum experienced significant morbidity throughout the challenge period (FIG. 20B). All control mice (survival 0/6) died 9 days after ZIKV challenge and had significantly lower survival (p=0.0016) than mice administered ZIKVLPs (survival 5/5, FIGS. 2B and C). Finally, ZIKVLPs vaccinated mice had significantly lower levels of viremia on day 2 post challenge than control mice detected by qRT-PCR (ZIKVLP=1.3−10⁴ RNA copies, PBS/alum 9.6×10⁷ RNA copies, p=0.0356, FIG. 2D) and TCID₅₀ assay (ZIKVLP=1.3×10² TCID₅₀s, PBS/alum 2.8×10⁵ TCID₅₀s p=0.0493, FIG. 2E).

ZIKVLPs Elicit Plaque Reducing Neutralizing Antibody Titers in Mice that can be Passively Transferred to Naïve Mice.

The plaque reduction neutralization test (PRNT) assay is widely considered to be the “gold standard” for characterizing and quantifying circulating levels of anti-dengue and other flaviviral neutralizing antibodies (nAb) (Thomas et al., 2009). A PRNT assay was developed for rapidly measuring ZIKV specific neutralizing antibodies. Pooled serum samples collected from mice pre-challenge, as well as individual serum samples collected from mice post-challenge were tested by this PRNT assay. Pre challenge, pooled serum from mice administered ZIKVLPs had a calculated 50% plaque reduction (PRNT₅₀) titer of 1:157. The PRNT₅₀ titer increased 2 weeks post challenge (GMT=5122) (FIG. 2F).

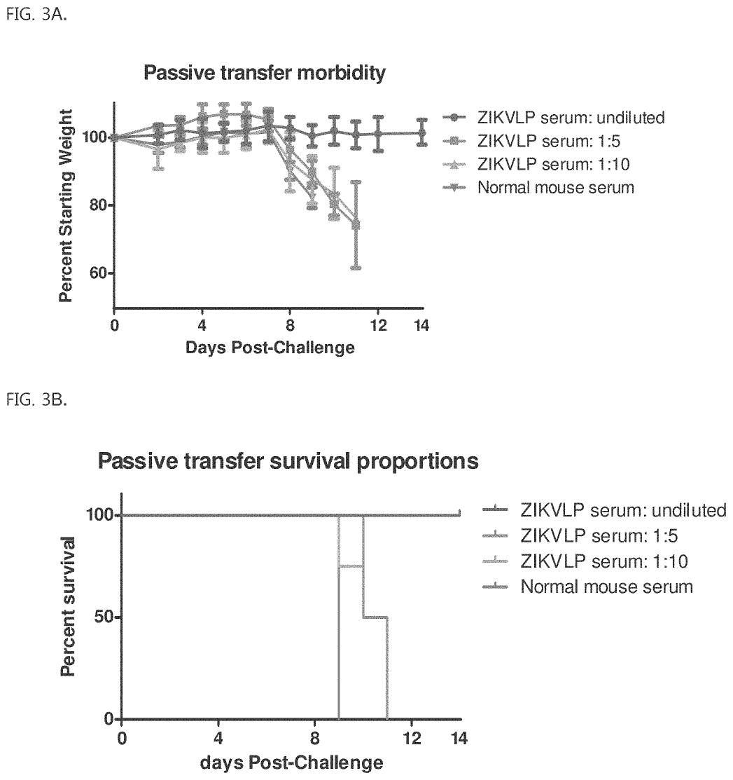

To test the role of anti-ZIKV antibodies in protection against challenge, groups of mice received ZIKVLP antiserum (pooled pre challenge serum, titer in FIG. 2F), undiluted (n=5), diluted 1:5 (n=4), or 1:10 (n=4). As a negative control, mice (n=5) were transferred serum from mice previously vaccinated with PBS alum. Negative control mice rapidly lost weight starting after day 7 and all died day 9 post challenge (FIGS. 3A-B). Mice that received undiluted serum maintained weight throughout the 14 day period post challenge, and showed no signs of infection. Mice that received diluted anti-ZIKV antibodies were not protected from challenge, although survival and weight loss were slightly extended relative to negative control mice (FIGS. 3A-B).

### A Single Dose of ZIKVLPs can Protect Highly Susceptible AG129 Mice

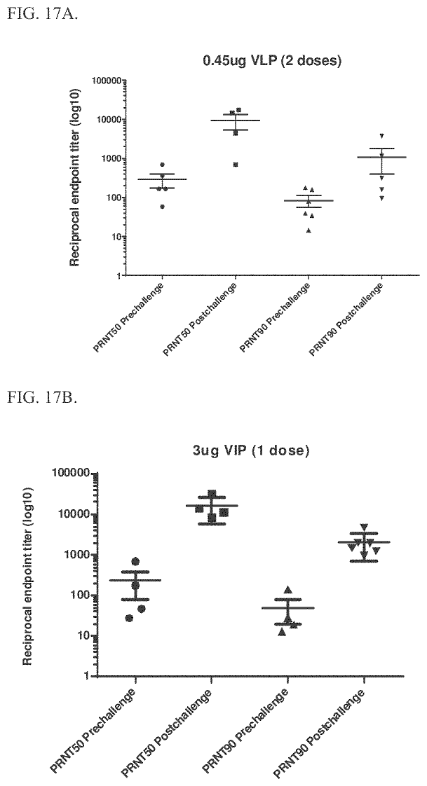

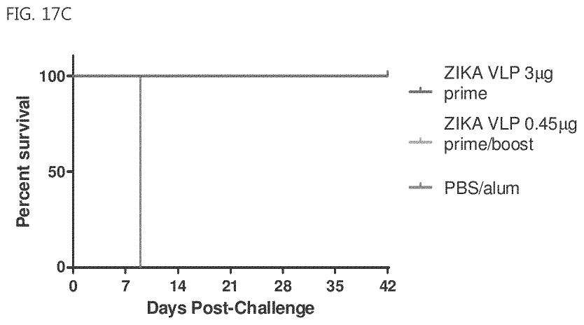

To determine if a single dose could protect AG129 mice, groups of 6-week old AG129 mice were vaccinated with 3 μg ZIKVLPs adjuvanted with alum. An additional group of mice (n=5) was vaccinated with a prime and boost of 0.45 μg adjuvanted with alum for comparison. Negative control mice (n=5) received a prime and boost of PBS/alum. Vaccinated mice developed neutralizing antibodies measured by PRNT assay prior to challenge (FIG. 17A). Eight weeks following primary vaccination mice were challenged with 200 PFU (>1000 LD₅₀s) of ZIKV by the ID route. All mice administered a prime of 3 μg or a prime and boost of 0.45 μg ZIKVLPs survived throughout the 6 week challenge period (FIG. 17C) and maintained weight throughout the challenge period. Pre challenge neutralizing antibody titers in both single (GMT PRNT50=288, PRNT90=81) and double dose (GMT PRNT50=235, PRNT90=50) groups increased significantly (p<0.005) in all animals measured at 3 weeks post challenge (FIGS. 17A-B).

### ZIKVLPS Protect Wildtype BALB/c Mice

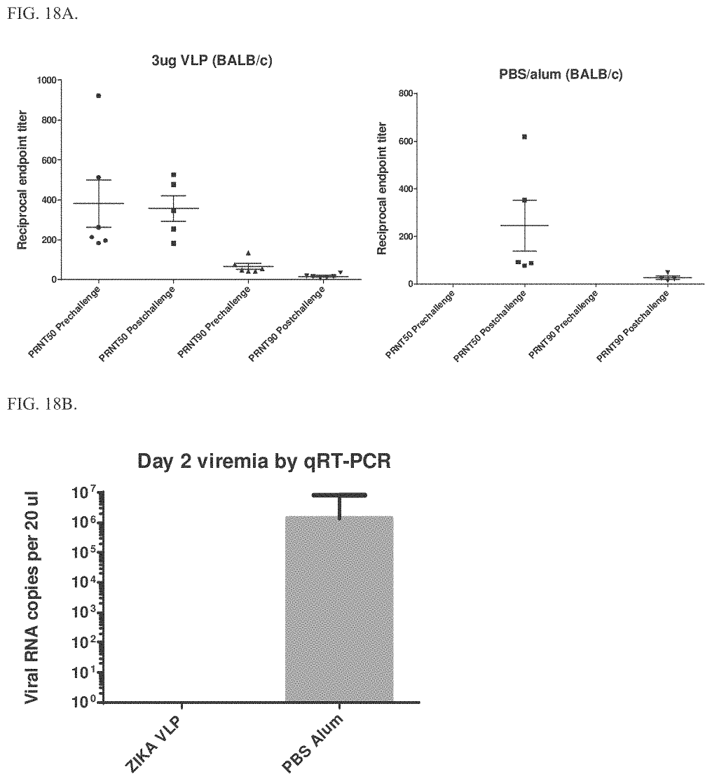

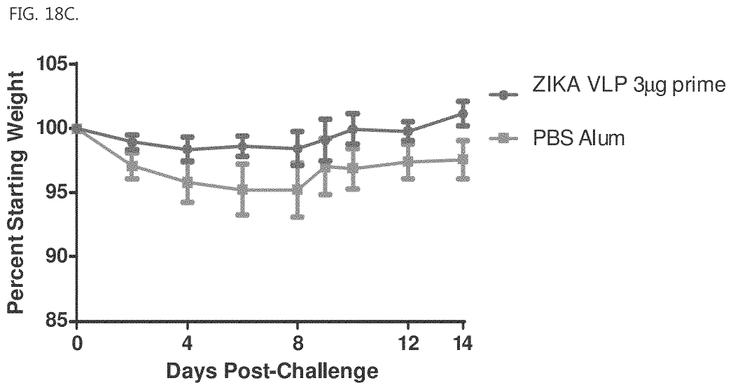

To determine if ZIKVLPs can protect wildtype BALB/c mice against non-lethal ZIKV challenge, a group (n=6) was vaccinated with a single dose of 3 μg ZIKVLPS adjuvanted with alum. Negative control mice (n=5) were administered PBS/alum. Eight weeks after vaccination mice were challenged with 200 PFU ZIKV by the IV route. A single dose of ZIKVLPs elicited high titers of neutralizing antibodies (PRNT50=381, PRNT90=75) detected immediately prior to challenge (FIG. 22A). Mice vaccinated with ZIKVLPS were completely protected from viremia on day 2 post challenge (FIG. 18B), and maintained weight throughout the challenge period (FIG. 18C). Negative control animals lost minor amounts of weight beginning at day 2 post challenge, had high levels of viremia and recovered by 2 weeks post challenge. Neutralizing antibodies were undetectable in negative control mice prior to challenge, but increased significantly after challenge (FIG. 18A). Antibody titers in vaccinated mice decreased, but were not significantly different than before ZIKV challenge (FIG. 18A).

### Discussion

Most experts and public health workers agree that a Zika vaccine is urgently needed. In February 2016, the World Health Organization declared that the recent clusters of microcephaly and other neurological disorders in Brazil constitute a public health emergency of international concern. Their recommendations included enhanced surveillance and research, as well as aggressive measures to reduce infection with Zika virus, particularly amongst pregnant women and women of childbearing age. ZIKV is now receiving considerable attention due to its rapid spread in the Americas, and its association with microcephaly (Mlakar et al., 2016) and Guillain-Barre syndrome (Pinto Junior et al., 2015). In these studies, a ZIKV-virus-like particle (VLP) vaccine was designed and it was expressed in vitro as shown by western blot and transmission electron microscopy, and its protective efficacy and role of antibodies in protection in the AG129 mouse model tested. An overall yield of 2.2 mg/L was calculated for the VLP tested. Similar expression levels have been reported for other flavivirus VLP expression strategies (Pijiman, 2015). Future work will optimize VLP production and purification parameters, which should significantly increase both yield and purity. Stably transfected HEK cells that continuously express VLPs allow for scalable production to help meet global demand for a ZIKV vaccine, which is estimated to be 100 million doses a year.

ZIKV-VLPs, formulated with alum, induced detectable neutralizing antibodies and protected animals against lethal challenge (>400 LD₅₀s) with no morbidity or mortality. Pre-challenge GMT neutralizing titers were 1:32, and pooled pre-challenge serum PRNT₉₀ and PRNT₅₀ titers were 1:34 and 1:157 respectively. At a relatively low dose of 450 ng, our results indicate that our ZIKVLPs are highly immunogenic. The antibody titers obtained are consistent with those reported for other highly immunogenic flavivirus VLP vaccines (Ohtaki et al., 2010; Pijlman, 2015). Previous work has shown a direct correlation between dose of VLPs and neutralizing antibody titers. For ZIKV, questions remain about the quantitative relationship between dose of VLPs and their effect on neutralizing antibody titers and protection from ZIKV challenge in vivo.

In the above-described studies, mice were vaccinated with ZIKVLPS and challenged with a homologous strain of ZIKV (H/PF/2013), which raises the question of ZIKVLP specific antibody cross reactivity to heterologous viruses currently circulating in the Americas. Although the H/PF/2013 virus was isolated well before the current outbreak from a patient infected in French Polynesia, there is a high degree of amino acid similarity (about 99%) to endemic South American strains of ZIKV (Faria et al., 2016; Zanluca et al., 2015). Some experts agree that the high serological cross-reactivity among ZIKV strains would allow for a monovalent vaccine (Lazear and Diamond, 2016). Nevertheless, care must be taken to empirically determine if antibody responses elicited by ZIKVLPs cross-react and protect against South American strains. Finally, any future ZIKV vaccination programs should incorporate careful surveillance of circulating strains to help suppress immunological escape, and ensure efficacy of vaccines in human populations.

Vaccinated AG129 mice challenged with >1000 LD₅₀s had low levels of viremia (1.3×10² TCID₅₀s, FIG. 2E) detected after challenge. Copies of RNA ZIKV genomes in serum of mice were significantly higher than levels of viremia. However, the disparity between viral genome copies and viremia has been observed for other flaviviruses including dengue (Bae et al, 2003). Since AG129 mice are highly susceptible to viral challenge, it is possible that the challenge dose given for the active vaccination study was artificially high. Methods for challenging mice from infected mosquito bite should be developed to most accurately mimic natural infection. The most important criteria for any ZIKV vaccine is its ability to prevent placental and fetal pathology in ZIKV infected pregnant women. Recently developed IFN deficient pregnant mouse models can provide an opportunity to assess if vaccination of pregnant animals can protect the fetus from ZIKV-induced pathology. (Miner et al., 2016). Although models for ZIKV infection in pregnant non-human primates (NHP) are still being developed, ZIKV vaccines should be tested in NHP translational models which most accurately mimics human immune responses to vaccination.

A VLP vaccine approach against ZIKV has significant advantages over other technologies as it will eliminate concerns of live attenuated vaccines and insufficient inactivation of killed vaccines for pregnant women and other populations at high risk of suffering the devastating effects of ZIKV infections. Production of inactivated vaccines requires high titer growth of infectious virus which may pose a safety concern for workers. Additionally, the production of both attenuated and inactivated ZIKV vaccines is limited to “batch” production, whereas flavirus VILPs can continuously expressed from stable cell lines. In recent years, recombinant virus-like particle (VLP)-based vaccine strategies have been frequently used for vaccine design. VLPs are known to be highly immunogenic and elicit higher titer neutralizing antibody responses than subunit vaccines based on individual proteins (Ariano et al., 2010).

The role of neutralizing antibodies in protecting against ZIKV was demonstrated by antibody passive transfer studies as naive AG129 mice receiving pooled serum from VLP vaccinated animals were fully protected. These results are consistent with previous findings that indicate the important role of antibodies in protecting against many insect-borne flaviviruses, such as Japanese encephalitis, west Nile virus, and tick borne encephalitis (Chiba et al., 1999; Kimura-Kuroda and Yasui, 1988; Tesh et al., 2002), even at low levels of circulating antibodies. In this study, full protection was observed when animals received undiluted serum (PRNT₅₀ 1:157), with no weight loss or other clinical signs observed. While these studies highlight the importance of serum antibodies in ZIKV protection, there are still many important questions related to ZIKV immunology. What is the minimum antibody titer needed for protection, do ZIKVLPs elicit CD8+ responses and are these responses involved in protection, and what is the overall role of cellular immunity in protection? It is also important to determine if anti-ZIKV antibodies, particularly those elicited by ZIKVLPs, play any role in dengue protection or disease enhancement.

In this study AG129 IFN receptor-deficient mice were used. This mouse models are commonly used for the evaluation of arboviral vaccines, including dengue, chikungunya and yellow fever virus (Meier et al., 2009; Partidos et al., 2011; Prestwood et al., 2012). We recently documented the suitability of mice deficient in IFN-α/β and -γreceptors as an animal model for ZIKV, as they are highly susceptible to ZIKV infection and disease, developing rapid viremic dissemination in visceral organs and brain and dying 7-8 days post-infection (Aliota et al., 2016), and evaluated doses as low as 1 PFU. In our current studies we observed consistent lethality at doses below 1 PFU, indicating that there are viral subpopulations refractory for the formation of CPE in cell culture, but still capable of establishing a lethal infection in highly susceptible mice. It is of great interest is that at a very low dose (0.2 PFU) two of five mice became ill more than 1 month after infection, as infection with ZIKV typically produces rapid lethality in AG129 mice.

The current studies challenged mice with 200 PFU at 11 weeks of age. All control mice lost 20% weight, were moribund, and succumbed to by challenge by day 9. ZIKV challenge therefore appears to be completely lethal in both juvenile and adult AG129 mice. The AG129 mouse model exhibits an intact adaptive immune system, despite the lack of an IFN response, and it has been used extensively to evaluate vaccines and antivirals for DENV (Brewoo et al., 2012; Fuchs et al., 2014; Johnson and Roehrig, 1999; Sarathy et al., 2015). In our studies WT BALB/c mice did not succumb to infection with ZIKV consistent with previous studies where BALB/c mice were experimentally inoculated with 200 PFU of ZIKV (Larocca et al., 2016). Mice also developed high levels of viremia following IV inoculation. A single dose of VLPs prevented detection of viral RNA copies in serum of vaccinated mice at 2 days post infection—when viremia levels typically peak in the BALB/c model. It is possible that viral replication was completely inhibited, as there was no “boost” response in neutralizing antibodies observed following challenge. Finally, in repeat AG129, and Balb/c mice mouse studies, animals were protected from ZIKV challenge 8 weeks after vaccination. ZIKVLP therefore appear to elicit a potent “memory” response.

In the present study, aluminum hydroxide (commonly known as alum) was used as the adjuvant for ZIKV-VLP preparations. Since its first use in 1932, vaccines containing aluminum-based adjuvants have been successfully administered in humans demonstrating excellent safety. Adjuvant formulations of ZIKV-VLP may facilitate antigen dose sparing, enhanced immunogenicity, and broadened pathogen protection.

In summary, a vaccine against ZIKV is currently unavailable, nor is there any specific prophylactic treatment. A VLP based Zika vaccine that elicits protective antibodies in mice, and is safe, suitable for scalable production, and highly immunogenic, is disclosed herein. Fast-tracking development of this ZIKV vaccine is a public health priority and is crucial for restoring confidence and security to people who wish to have children or reside in, or visit areas in which ZIKV is endemic.

### REFERENCES

Akahata et al., Nat. Med., 16:334 (2010).

Aliota et al., PLoS Negl. Trop. Dis., 10:e0004682 (2016).

Ariano et al., CMAJ, 182:357 (2010).

Rae et al., J. Virol. Methods, 110:185 (2003).

Brewoo et al., Vaccine. 30:1513 (2012).

Butler, Nature, 531:153 (2016).

Cao-Lormeau et al., Emerg. Infect. Dis., 20:1085 (2014).

Chiba et al., Vaccine, 17:1532 (1999).

Duffy et al., N. Engl. J. Med., 360:2536 (2009).

Dyer, BMJ, 351:h6983 (2015).

Etna et al., Science. 352:345 (2016).

Faye et al., PLoS Negl. Trop. Dis., 8:e2636 (2014).

Fuchs et al., Vaccine, 32:6537 (2014).

Gaskell et al., Emerg. Infect. Dis., 23:137 (2017).

Guliand, BMJ, 352:i657 (2016).

Hennessey et al. Am. J. Trop. Med. Hyg., 95:212. (2016).

Honibach et at, Bmj. 355:i5923 (2016).

Honein et al., Jama. 317:59 (2017).

Ioos et al., Med. Mal. Infect., 44:302 (2014).

Johnson et al., J. Virol., 73:783 (1999).

Kimura-Kuroda et al., J. Immunol., 141:3606 (1988).

Lanciotti et al., Emerg. Infect. Dis., 14:1232 (2008).

Larocca et al., Nature, ______:______ (2016).

Lazear et at, J. Virol., 90:4864 (2016).

Li et al., Neuron., 92:949 (2016).

Meier et al., PLoS Pathog., 5:e1000614 (2009).

Merino-Ramos et al., PLoS One, 9:e108056 (2014).

Metz et al., Methods Mol. Biol., 1426:297 (2016).

Miner et al., Cell. 165:1081 (2016).

Mlakar et al., N. Engl. J. Med., 374:951 (2016).

Musso, Emerg. Infect. Dis., 21:1887 (2015).

Ohtaki et al., Vaccine, 28:6588 (2010).

Oliveira Melo et al., Ultrasound Obstet. Gynecol., 47:6 (2016).

Partidos et al., Vaccine, 29:3067 (2011).

Pijlman, Biotechnol. J., 10:659 (2015).

Pinto Junior et al., Acta Med. Port., 28:760 (2015).

Prestwood et al., J. Virol., 86:12561 (2012).

Reed et at, Am. J. Epid., 27:493 (1938).

Sarathv et al., J. Gen. Virol., 96:3035 (2015).

Shawan et al., Nat. Sci., ______:37 (2015).

Spohn et al., Virol. J., 7:146 (2010).

Tesh et, al., Emerg. Infect. Dis., 8:1392 (2002).

Thomas et al., Am. J. Trop. Med. Hyg., 81:825 (2009).

Ticconi et al., Pathog, Glob. Health, 110:262 (2016).

Wang et al. Vaccine, 30:2125 (2012).

Wang et al., Biomed. Res. Int., 2013:686549 (2013).

Zanluca et al., Mem. Inst. Oswaldo Cruz, 110:569 (2015).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A recombinant nucleic acid vector comprising a heterologous promoter operably linked to a nucleotide sequence encoding flavivirus prM/E, which vector lacks nucleic acid sequences encoding one or more of flavivirus NS1, NS2A, NS2B, NS3, NS4A, NS4B or NS5 and optionally lacks nucleic acid sequences encoding functional flavivirus capsid.

2. The recombinant vector of claim 1 wherein the heterologous promoter is a heterologous viral promoter.

3. The recombinant vector of claim 1 which includes a portion of flavivirus capsid sequences.

4. The recombinant vector of claim 1 wherein the capsid sequence includes amino acids 98 to 112 of the capsid protein encoded by SEQ ID NO:1 or a protein having at least 80% amino acid sequence identity thereto.

5. The recombinant vector of claim 1 wherein the flavivirus is a Zika virus.

6. The recombinant vector of claim 1 wherein the prM/E sequences have at least 80% amino acid sequence identity to the prM/E sequences encoded by any one of SEQ ID Nos. 1-3 or 5.

7. The recombinant, vector of claim 1 wherein the prM/E sequences are operably linked to a heterologous secretion signal.

8. The recombinant vector of claim 7 wherein the heterologous secretion signal is a TPA, IL-2, IgG kappa light chain, CD33, or Oikosin secretion signal.

9. A vaccine comprising an effective amount of a flavivirus like particle comprising a lipid bilayer comprising flavivirus prM/E but which particle lacks one or more of flavivirus NS1, NS2A, NS2B, NS3, NS4A, NS4B NS5 and optionally lacks functional flavivirus capsid.

10. The vaccine of claim 9 further comprising one or more adjuvants.

11. The vaccine of claim 10 wherein the adjuvant comprises alum, monophosphoryl lipid A (MPLA), squalene, aluminum hydroxide absorbed TLR4 agonist, dimethyldioctadecylammonium, tripalmitoyl-S-glyceryl cysteine, trehalose dibehenate, saponin, MF59, AS03, virosomes, AS04, CpG, imidazoquinoline, poly I:C, flagellin, or any combination thereof.

12. The vaccine of claim 9 wherein the flavivirus is a Zika virus.

13. The vaccine of claim 9 wherein the prM/E sequences have at least 80% amino acid sequence identity to the prM/E sequences encoded by any one of SEQ II) Nos. 1-3 or 5.

14. A method to prevent, inhibit or treat flavivirus infection in a mammal, comprising: administering to the mammal a composition comprising an effective amount of a flavivirus like particle comprising a lipid bilayer comprising flavivirus prM/E but which particle lacks one or more of flavivirus NS1, NS2A, NS2B, NS3, NS4A, NS4B or NS5 and optionally lacks functional flavivirus capsid, or a composition comprising an effective amount of anti-flavivirus antibodies.

15. The method of claim 14 wherein the malmnal is a female mammal.

16. The method of claim 14 wherein the mammal is a human.

17. The method of claim 14 wherein the flavivirus is a Zika virus.

18. The method of claim 17 wherein the prM/E sequences have at least 80% amino acid sequence identity to the prM/E sequences encoded by any one of SEQ ID Nos. 1-3 or 5.

19. The method of claim 14 wherein the composition comprising the flavivirus like particle is administered intramuscularly, subcutaneously or intranasally.

20. The method of claim 14 wherein the composition inhibits flavivirus infection.

21. (canceled)

22. (canceled)


SEQUENCE LISTING (14 sequences)

SEQ ID NO: 1 - DNA, 2601 bp, Zika virus
atcaggtgcataggagtcagcaatagggactttgtggaaggtatgtcaggtgggacttgggttgatgtcgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgttgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaggaaggagcagttcacacggcccttgctggagctctggaggctgagatggatggtgcaaagggaaggctgtcctctggccacttgaaatgtcgcctgaaaatggacaaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgtggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttctgggctaaagatggctgttggtatggaatggagataaggcccaggaaagaaccagaaagcaacttagtaaggtcaatggtgactgcaggatcaactgatcacatggatcacttctccctt

SEQ ID NO: 2 - DNA, 10807 bp, Zika virus
agttgttgatctgtgtgaatcagactgcgacagttcgagtttgaagcgaaagctagcaacagtatcaacaggttttattttggatttggaaacgagagtttctggtcatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagtttaagaaagatctggctgccatgctgagaataatcaatgctaggaaggagaagaagagacgaggcacagatactagtgtcggaattgttggcctcctgctgaccacagccatggcagtggaggtcactagacgtgggaatgcatactatatgtacttggacagaagcgatgctggggaggccatatcttttccaaccacaatggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccaccacaaaaaaggtgaagcacggagatctagaagagctgtgacgctcccctcccattccactaggaagctgcaaacgcggtcgcagacctggttggaatcaagagaatacacaaagcacctgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgttaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcttgctctaagaaaatgaccgggaagagcatccagccagagaatctggagtaccggataatgctgtcagttcatggctcccagcacagtgggatgatcgttaatgatacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcatgctggggcagacaccggaactccacactggaacaacaaagaagcactggtagagttcaaggacgcacatgccaaaaggcagactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcactaagatcccggctgaaacactgcacgggacagtcacagtggaggtacagtacgcagggacagatggaccttgcaaggttccagctcagatggcggtggacatgcaaactctgaccccagttgggaggttgataaccgctaaccctgtaatcactgaaagcactgagaactccaagatgatgctggaactggatccaccatttggggactcttacattgtcataggagtcggggaaaagaagatcacccaccactggcacaggagtggcagcaccattggaaaagcatttgaagccactgtgagaggtgccaagagaatggcagtcttgggagacacagcctgggactttggatcagttgggggtgctctcaactcactgggcaagggcatccatcaaatttttggagcagctttcaaatcattgtttggaggaatgtcctggttctcacaaattctcattggaacgttgctggtgtggttgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggaacttcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctataagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 3 - DNA, 10272 bp, Zika virus
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagccttcttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactaatgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacttggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggctgataaccgctaaccccgtaatcactgaaagcactgagaactccaagatgatgctggaacttgatccaccatttggggactcttacattgtcataggagtcggggagaagaagatcacccaccactggcacaggagtggcagcaccattggaaaagcatttgaagccactgtgagaggtgccaggagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcaccaaggtccacgtggaggaaacatgtggaacaagaggaccatctctgagatcaaccacagcaagcggaagggtgatcgaggaatggtgctgcagggagtgcacaatgcccccactgtcgttccaggctaaagatggctgttggtatggaatggagataaggcccaggaaagaaccagaaagtaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggcttgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggagctctatgggatgtgcctgctcccaaggaagtaaaaaagggggagaccacagatggagtgtacagagtgatgactcgtagactgctaggttcaacacaagttggagtgggagttatgcaagagggggtctttcacaccatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgccgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtctggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcacgcggccctgaagtcattcaaggagtttgccgctgggaaaagaggagcggcttttggagtgatggaagccttgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctacgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtagccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagctgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctttccatggtgggggattggcttgaaaaaagaccaggagccttttgtataaaagtgttgtgtccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagatccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcgagacaagaccaaagggggagcggacaagttgtcacttacgctcttaacacatttaccaacctagtggtgcaactcattcggagtatggaggctgaggaagttctagagatgcaagacttgtggctgctgcggaggtcagagaaagtgaccaactggctgcagagcaacggatgggataggctcaaacgaatggcagtcagtggagatgattgcgttgtgaggccaattgatgataggtttgcacatgccctcaggttcttgaatgatatggggaaagttaggaaggacacacaagagtggaaaccctcaactggatgggacaactgggaggaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 4 - DNA, 392 bp, Artificial Sequence
  A synthetic oligonucleotide
atcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaac

SEQ ID NO: 5 - DNA, 4251 bp, Artificial Sequence
  A synthetic oligonucleotide
  misc_feature (1)...(4251): n = A,T,C or G
tgttgatactcatactcttcctttttcatattattgaagcatttatcagggttattgtctcatgagccnnnnnnnnnnnnnnnnnnnnnnnnnnnnnnnnanaantnngggttccccgcncattnccccgaaaagtgccacctgacgtcgncggatcgggagatctccnnnnnnnnnnnnnnnnnnnnnnnnnnnnnnnngctctgatgccgcatagttaagccagtatctgctccctgcttgtgtgttggaggtcgctgagtagtgcnnnnnnnnnnnnnnnnnnnnnnnnnnnnnnnncttgaccgacaattgcatgaagaatctgcttagggttaggcgttttgcgctgcttcgcgatgtacggcnnnnnnnnnnnnnnnnnnnnnnnnnnnnnnnnagttattaatagtaatcaattacggggtcattagttcatagcccatatatggagttccgcgttacatannnnnnnnnnnnnnnnnnnnnnnnnnnnnnnncgaacgacccccgcccattgacgtcaataatgacgtatgttcccatagtaacgccaatagggactttcnnnnnnnnnnnnnnnnnnnnnnnnnnnnnnnnaactgcccacttggcagtacatcaagtgtatcatatgccaagtccgccccctattgacgtcaatgaccnnnnnnnnnnnnnnnnnnnnnnnnnnnnnnnnaactgcccacttggcagtacatcaagtgtatcatatgccaagtccgccccctattgacgtcaatgaccnnnnnnnnnnnnnnnnnnnnnnnnnnnnnnnnagcggtttgactcacggggatttccaagtctccaccccattgacgtcaatgggagtttgttttggcacnnnnnnnnnnnnnnnnnnnnnnnnnnnnnnnntaaccccgccccgttgacgcaaatgggcggtaggcgtgtacggtgggaggtctatataagcagagctcnnnnnnnnnnnnnnnnnnnnnnnnnnnnnnnncatccacgctgttttgacctccatagaagacaccgggaccgatccagcctccgcggccgggaacggtcnnnnnnnnnnnnnnnnnnnnnnnnnnnnnnnnactcaccgtccggatctcagcaagcaggtatgtactctccagggtgggcctggcttccccagtcaagannnnnnnnnnnnnnnnnnnnnnnnnnnnnnnnctcttacatgtaccttttgcttgcctcaaccctgactatcttccaggtcaggatcccagagtcagggcnnnnnnnnnnnnnnnnnnnnnnnnnnnnnnnngaacagtaaaccctgctccgaatattgcctctcacatctcgtcaatctccgcgaggactggggaccctnnnnnnnnnnnnnnnnnnnnnnnnnnnnnnnncctcctgctgaccacagctatggcagcggaggtcactagacgtgggagtgcatactatatgtacttgcnnnnnnnnnnnnnnnnnnnnnnnnnnnnnnnnccaaccacattggggatgaataagtgttatatacagatcatggatcttggacacatgtgtgatgccacnnnnnnnnnnnnnnnnnnnnnnnnnnnnnnnngggtggaaccagatgacgtcgattgttggtgcaacacgacgtcaacttgggttgtgtacggaacctgcnnnnnnnnnnnnnnnnnnnnnnnnnnnnnnnnaagagctgtgacgctcccctcccattccactagtttgctgcaaacgcggtcgcaaacctggttggaatnnnnnnnnnnnnnnnnnnnnnnnnnnnnnnnngaaaattggatattcaggaaccctggcttcgcgttagcagcagctgccatcgcttggcttttgggaatnnnnnnnnnnnnnnnnnnnnnnnnnnnnnnnntgatactgctgattgccccggcatacagcatcaggtgcataggagtcagcaatagggactttgtggaannnnnnnnnnnnnnnnnnnnnnnnnnnnnnnncttggaacatggaggttgtgtcaccgtaatggcacaggacaaaccgactgtcgacatagagctggttannnnnnnnnnnnnnnnnnnnnnnnnnnnnnnntcctactgctatgaggcatcaatatcggacatggcttcggacagccgctgcccaacacaaggtgaagcnnnnnnnnnnnnnnnnnnnnnnnnnnnnnnnntctgcaaaagaacgttagtggacagaggctggggaaatggatgtggactttttggcaaagggagcctcnnnnnnnnnnnnnnnnnnnnnnnnnnnnnnnnaatgaccgggaagagcatccagccagagaatctggagtaccggataatgctgtcagttcatggctcccnnnnnnnnnnnnnnnnnnnnnnnnnnnnnnnncatgaaactgatgagaatagagcgaaggttgagataacgcccaattcaccaagagccgaagccaccctnnnnnnnnnnnnnnnnnnnnnnnnnnnnnnnnaaccgaggacaggccttgacttttcagatttgtattacttgactatgaataacaagcactggttggttnnnnnnnnnnnnnnnnnnnnnnnnnnnnnnnnttggcacgctggggcagacaccggaactccacactggaacaacaaagaagcactggtagagttcaagcnnnnnnnnnnnnnnnnnnnnnnnnnnnnnnnnctagggagtcaagaaggagcagttcacacggcccttgctggagctctggaggctgagatggatggtgcnnnnnnnnnnnnnnnnnnnnnnnnnnnnnnnngtcgcctgaaaatggataaacttagattgaagggcgtgtcatactccttgtgtaccgcagcgttcacannnnnnnnnnnnnnnnnnnnnnnnnnnnnnnngacagtcacagtggaggtacagtacgcagggacagatggaccttgcaaggttccagctcagatggcgcnnnnnnnnnnnnnnnnnnnnnnnnnnnnnnnnttgataaccgctaaccccgtaatcactgaaagcactgagaactctaagatgatgctggaacttgatccnnnnnnnnnnnnnnnnnnnnnnnnnnnnnnnntcggggagaagaagatcacccaccactggcacaggagtggcagcaccattggaaaagcatttgaagccnnnnnnnnnnnnnnnnnnnnnnnnnnnnnnnngggagacacagcctgggactttggatcagttggaggcgctctcaactcattgggcaagggcatccatcnnnnnnnnnnnnnnnnnnnnnnnnnnnnnnnnggaggaatgtcctggttctcacaaattctcattggaacgttgctgatgtggttgggtctgaacacaaannnnnnnnnnnnnnnnnnnnnnnnnnnnnnnntagggggagtgttgatcttcttatccacagctgtctctgctgatgtggggtgctcggtgtgaggatctnnnnnnnnnnnnnnnnnnnnnnnnnnnnnnnnccctaaacttcatgggttacgtaattggaagttgggggacattgccacaagatcatattgtacaaaacnnnnnnnnnnnnnnnnnnnnnnnnnnnnnnnncaggcctattgattggaaagtatgtcaaaggattgtgggtcttttgggctttgctgctccatttacacnnnnnnnnnnnnnnnnnnnnnnnnnnnnnnnngcatgtatacaagctaaacaggctttcactttctcgccaacttacaaggcctttctaagtaaacagtannnnnnnnnnnnnnnnnnnnnnnnnnnnnnnnctggtctgtgccaagtgtttgctgacgcaacccccactggctggggcttggccataggccatcagcgcnnnnnnnnnnnnnnnnnnnnnnnnnnnnnnnnccatactgcggaactcctagccgcttgttttgctcgcagccggtctggagcaaagctcataggaactcnnnnnnnnnnnnnnnnnnnnnnnnnnnnnnnntcgtttcgatctacgtatgatctttttccctctgccaaaaattatggggacatcatgaagccccttgannnnnnnnnnnnnnnnnnnnnnnnnnnnnnnnttttcattgcaatagtgtgttggaattttttgtgtctctcactcggaaggaattctgcattaatgaatnnnnnnnnnnnnnnnnnnnnnnnnnnnnnnnnttgggcgctcttccgcttcctcgctcactganctcgntgcgcttcggtcgt

SEQ ID NO: 6 - protein, 867 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFWAKDGCWYGMEIRPRKEPESNLVRSMVTAGSTDHMDHFSL

SEQ ID NO: 7 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGTDTSVGIVGLLLTTAMAVEVTRRGNAYYMYLDRSDAGEAISFPTTMGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAAKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVPINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMAICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRYGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMMRRIIGDEEKYVDYLSTQVRYLGEEGSTPGVL

SEQ ID NO: 8 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTNVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGARRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFQAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRSMEAEEVLEMQDLWLLRRSEKVTNWLQSNGWDRLKRMAVSGDDCVVRPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 9 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 10 - protein, 6 aa, Zika virus
KEKKRR

SEQ ID NO: 11 - DNA, 10251 bp, Zika virus
atgaaaaacccaaagaagaaatccggaggattccggattgtcaatatgctaaaacgcggagtagcccgtgtaaaccccttggggggtttgaagaggctgccggccggactcctgctgggccatggacccatcagaatggttttggcgatactagccttcttgagattcacagcaatcaagccatcactgggcctcatcaatagatggggttccgtggggaagaaggaggctatggaaataataaaaaagttcaagaaagatcttgctgccatgttgagaataatcaatgctaggaaggagaggaagagacgtggagctgatgccagcatcggaatcgtcagcctcctgctgactacagtcatggcagcagagatcactagacgcgggagtgcatactacatgtacttggacaggagcgatgctggtaaggccatttctttcgttaccacactgggggtgaacaaatgccatgtgcagatcatggacctcgggcatatgtgtgacgccaccatgagttatgagtgccccatgctggacgagggagtggagccagatgacgtcgattgctggtgcaacacgacatcaacttgggttgtgtacggaacctgtcatcataaaaaaggtgaagcacgacgatccagaagagccgtgacgcttccttctcactctacaaggaagttgcaaacgcgatcgcagacttggctagaatcaagagaatacacaaagcacctgatcaaggttgagaattggatattcaggaaccccgggtttgcgctagtggctgtagctattgcctggctcctgggaagctcgacgagccaaaaagtcatatacttggtcatgatattgttgattgccccggcatacagtatcaggtgcataggagttagcaatagagacttcgtggagggcatgtcaggtgggacctgggttgatgttgtcttggaacatggaggttgtgtcaccgtgatggcacaggacaagccaacagttgacatagagttggtcacgacaacggttagcaacatggccgaggtgagatcctactgctacgaggcatcaatatcggacatggcttcggacagtcgctgcccaacacaaggtgaagcctaccttgacaagcagtcagacactcaatatgtctgtaaaagaacattggtggacagaggttggggaaatgggtgtggactttttggcaaggggagcttggtgacgtgtgccaagtttacatgctccaagaaaatgacagggaagagcatccagccggagaacttggagtaccggataatgctatcagtgcatggatcccagcacagtgggatgattgtgaatgacgaaaacagagcaaaagtcgaggttacacccaattcaccaagagcagaagcaaccttgggaggttttggaagcctgggacttgattgtgaaccaaggacaggccttgacttttcagatctgtattacctgaccatgaacaataagcattggttggtgcacaaagagtggtttcatgacatcccattaccttggcattctggtgcagacactgaaactccacactggaacaacaaagaggcactggtggagttcaaggacgcccacgccaagaggcaaactgttgtggttctggggagccaagaaggagccgttcacacggctctcgctggagctctggaggctgagatggatggtgcgaagggaaggctatcctcaggccatttgaaatgccgcctaaaaatggacaagcttaggttgaagggtgtgtcatattccctgtgtaccgcagcgttcacattcaccaaggttccagctgaaacattgcatggaacagtcacagtggaggtgcagtatgcagggagggatggaccctgcaaggtcccagcccagatggcggtggacatgcagaccctgaccccagttggaaggctgataacggctaaccctgtgatcactgaaagcactgagaattcaaagatgatgttggagctcgacccaccatttggggattcttacattgtcataggagtcggggacaagaaaatcacccatcactggcatcggagtggtagcatcatcggaaaggcatttgaagccactgtgagaggcgccaagagaatggcagtcttgggagacacagcctgggactttggatcagttgggggtgtgtttaactcattgggcaagggtattcaccagatctttggagcagctttcaaatcactgttcggaggaatgtcctggttctcacagatcctcataggcacactgttggtgtggttgggtctgaacacaaagaatggatctatctccctcacatgcttggccttgggaggagtgatgatcttcctttccacggctgtttctgctgatgtggggtgttcggtggacttctcaaaaaaggaaacgagatgtggcacgggggtgttcatctacaatgacgttgaagcctggagggatcgatacagataccatcctgactccccccgcagattggcagcagctgttaagcaggcttgggaagaggggatttgtgggatctcctccgtttcgagaatggaaaacatcatgtggaaatcagtggaaggggagcttaatgcgatcctagaggagaatggagtccaactgacagttgtagtggggtctgtaaaaaaccccatgtggagaggtccacgaagattgccagtgcccgtaaatgagctgccccatggctggaaagcctgggggaaatcgtactttgttagggcggcaaagaccaacaacagttttgttgtcgacggtgacacactgaaggaatgtccgctcaaacatagagcatggaatagcttccttgtggaggatcacgggtttggggtcttccacaccagtgtttggctgaaggtcagagaggactattcattagagtgtgacccagccgtcataggaacagctgtcaagggaaaggaggctgcacacagtgatctaggctattggattgagagtgaaaagaatgacacatggaggctgaagagggctcatctgattgagatgaagacatgtgagtggccaaagtctcacacactgtggacagatggagtggaagaaagtgatctgatcatacccaagtccttagctggtccactcagccaccacaacaccagagagggttatagaactcaagtgaaagggccatggcatagtgaagagctcgaaatccggtttgaggaatgcccaggcaccaaggttcatgtggaggagacatgcggaactagaggaccatctttaagatcaaccactgcaagtggaagggtcatagaggaatggtgctgtagggaatgcacaatgcctccactatcgttccgggcaaaagacggctgctggtatggaatggagataaggcccagaaaggaaccagagagcaacttagtgaggtctatggtgacagcaggatcaaccgatcacatggatcacttctctcttggagtgcttgtgattctactcatggtgcaggaaggtttgaagaagagaatgaccacaaagatcataatgagcacatcaatggcaatgctggtagccatggtcttgggaggattctcaatgagtgacctggctaagcttgtgatcctgatgggtgccactttcgcagaaatgaacactggaggagatgtggctcacttggcattggtagcggcatttaaagtcagaccagccttgttggtttccttcatcttcagagccaactggacaccccgtgagagcatgctgctagccctggcttcgtgtctcctgcagactgcgatttccgctcttgaaggcgagctgatggtcctcgttaatggatttgctttggcctggttggcaatacgagcaatggccgtgccacgcactgataacatcgctctagcaattctggccgctctaacaccattagccagaggcacactgcttgtggcatggagagcgggcctcgccacttgtggagggttcatgctcatttccctgaaagggaaaggtagtgtgaagaagaacctgccatttgtcatggccttggggttgaccgctgtgaggatagtggaccccattaatgtggtaggactactgttactgacaaggagtgggaaacggagctggccccctagtgaagtgcttacagctgtcggcctgatatgtgcactggccggagggtttgccaaggcagacatagagatggctgggcccatggctgcagtaggcctgctaattgtcagttatgtggtcacgggaaagagtgtggacatgtacattgaaagagcaggtgatattacatgggaaaaagacgcggaagtcactggaaacagtcctcggcttgacgtggcactagatgagagtggtgatttctctttggtagaggaggatggcccacccatgagagagatcatactcaaggtggtcctgatggccatctgtggcatgaacccaatagccatacccttcgctgcaggagcgtggtatgtgtatgtaaagactgggaaaaggagcggtgccctctgggacgtgcctgctcccaaagaagtaaaaaagggagagactacagatggagtgtacagagttatgactcgcagactgctgggttcaacacaggttggagtgggagtcatgcaagagggagtcttccataccatgtggcacgtcacaaaaggagccgcattgaggagcggtgaaggaagacttgatccatactggggggacgtcaagcaggacctggtgtcatattgtgggccgtggaagttggatgcagcctgggatggactaagtgaggtgcagcttttggccgtaccccccggagagagggctaaaaacattcagactctgcctggaatatttaagacaaaggatggggacatcggagcagttgctctagactaccctgcaggaacctcaggatctccgatcctagacaaatgcggaagagtgataggactttatggcaatggggttgtgatcaagaatggaagctatgttagtgccataacccagggaaaaagggaggaggagactccggttgagtgctttgaaccctcgatgctgaggaagaagcagctaacagtcttggatctgcatccaggagccgggaaaaccaggagggttcttcctgaaatagtccgtgaagccataaagaagagacttcgcacagtgatcttagcaccaaccagggttgttgctgctgagatggaggaagccctaagaggacttccggtgcgttacatgacaacagcagtcaacgtcacccattctgggacagaaatcgttgatttgatgtgccatgccaccttcacttcacgcctactacaaccaatcagagtccccaactacaacctttatatcatggatgaggctcatttcacagatccttcaagcatagctgcaagaggatacatatcaacaagggttgaaatgggcgaggcggctgctatcttcatgactgctacaccaccaggaacccgcgatgcgtttccagattccaactcaccaatcatggacacagaagtggaagtcccagagagagcctggagctcaggctttgactgggtgacggaccattctggaaaaacaatttggtttgttccaagtgtgagaaacggaaatgaaatcgcagcctgtctgacaaaggctggaaagcgggttatacagctcagcaggaagacttttgagacagagtttcagaagacaaaaaatcaagagtgggactttgtcataacaactgacatttcagagatgggtgccaacttcaaggctgaccgggtcatagattccaggagatgcctaaagccagtcatacttgatggtgagagagtcatcctggctgggcctatgcccgtcacgcacgccagtgctgctcagaggagaggacgtataggcaggaaccccaacaaacctggagatgagtatatgtatggaggtgggtgtgcagagactgatgaagaccatgcacactggcttgaagcaagaatgcttctcgacaacatttacctccaggatggcctcatagcctcgctctatcggcctgaggctgacaaggttgccgccattgagggagagttcaagctgaggacagagcaaaggaagacctttgtggaactcatgaagagaggagaccttcccgtttggctggcctatcaagtagcatctgccggaataacttacacagacagaagatggtgctttgatggcactaccaacaacaccataatggaagacagtgtaccagcagaggtgtggaccaagtatggagagaagagagtgctcaaaccgaggtggatggatgccagggtctgttcagatcatgcggctttgaagtcgttcaaagaatttgccgctgggaagagaggagcggctttgggagtaatggatgccctaggaacattgccaggacacatgacagagaggtttcaggaagccattgacaatctcgctgtgctcatgcgagcagagactggaagtaggccctacaaagcagcggcagctcaactgccggagaccctagagaccattatgctcttgggtttattgggaacagtttcgctagggatcttctttgtcttgatgcggaacaagggcatcgggaagatgggcttcggaatggtaacccttggggccagcgcatggctcatgtggctttcggaaattgaaccagccagaatcgcatgtgtcctcattgtcgtgtttctgttactggtggtgctcatacctgagccagagaagcaaagatctccccaggacaatcaaatggcaatcatcatcatggtggcagtgggccttctgggtttgataactgcaaacgaactcggatggctggaaagaacaaaaagtgatatagctcatctaatgggaaggaaagaagaggggacaaccgtaggattctcaatggatattgatctgcggccagcctccgcctgggctatttatgccgcattgacaactctcatcaccccagccgtccaacatgcggtgaccacctcatacaacaactactccctgatggcgatggccacacaagctggagtgctgtttggcatgggcaaagggatgccattttatgcatgggactttggagtcccgctgctaatgatgggttgttactcacaattaacacccctgaccctgatagtggccatcattctgcttgtggcacactacatgtatttgatcccaggtttgcaggcagcagcagcacgtgccgcccagaagaggacagcagctggcatcatgaagaatcccgttgttgatggaatagtggtgactgacattgacacaatgacaattgacccccaagtggagaagaagatgggacaagtgttactcatagcagtagctgcctccagtgccgtgctgctgcggaccgcttggggatggggggaggctggggctctgatcacagcagcaacctccaccttatgggaaggctctccaaacaaatactggaactcctctacagccacttcactgtgcaatatcttcagaggaagttatttggcaggggcttcccttatttacacagtgacaagaaatgccggtctggttaagagacgtggaggtggaacgggagagactctgggagagaagtggaaagcccgcctgaaccagatgtcggctttggagttctattcttacaaaaagtcaggcatcaccgaagtgtgtagggaggaggcacgccgcgccctcaaggatggagtggccacaggaggacatgctgtatcccggggaagcgcaaagcttagatggttggtagagagaggatacctgcagccccatggaaaggttgttgacctcggatgtggcagagggggctggagttattacgctgccaccatccgtaaagtgcaggaggtcagaggatacacaaagggaggtcctggtcatgaagaacccatgctggtgcaaagctatgggtggaacatagttcgcctcaagagtggagtggacgtctttcacatggcggctgagccgtgtgacactttgctgtgtgacattggcgagtcatcgtccagtcctgaagtggaagagacgcgaacactcagagtgctctccatggtgggagactggctcgagaaaagaccaggggccttctgcataaaggtgctgtgcccatacaccagtactatgatggagaccatggagcgactgcaacgtaggtatgggggaggattggtcagagtgccattgtcccgcaactccacacatgagatgtattgggtctctggagccaaaagtaacatcataaagagtgtgtccaccacaagtcagctcctcttgggacgcatggatgggcctaggaggccagtgaaatatgaagaggatgtgaacctcggctcaggcacacgagctgtggcaagctgtgctgaggctcccaacatgaagatcattggtaggcgcattgagagaatccgcaatgaacatgcagagacatggttctttgatgaaaaccacccatacaggacatgggcctaccatgggagctacgaagcccccacgcaggggtcagcgtcatccctcgtgaacggggttgttagactcctgtcaaagccctgggatgtggtgactggagtcacaggaatagctatgactgacaccacgccatacggccaacaaagagtcttcaaagaaaaggtggacactagggtgccagacccccaagaaggcacccgccgagtaatgaacatggtctcgtcttggctatggaaggagctgggaaaacgcaagcggccacgtgtctgcaccaaagaagagttcatcaataaggtgcgcagcaatgcagcactgggagcaatatttgaagaggaaaaagaatggaagacagctgtagaagctgtgaatgatccgagattttgggctctagtggacaaggaaagagaacaccacctgagaggagagtgtcacagctgtgtgtacaacatgatgggaaaaagagaaaagaagcaaggagaattcgggaaagcaaaaggcagccgcgcaatctggtacatgtggttgggagccagatttctggagtttgaggctcttggattcttgaatgaggaccattggatgggaagagaaaactcaggaggtggcgttgaagggctaggactgcaaaggcttggatacattctagaagaaatgaaccgggcgccaggaggaaagatgtatgcagatgacaccgctggctgggatacccgtattagcaggtttgatctggagaatgaagccctgatcactaaccagatggaagaagggcacagagctctggcgttggccgtgattaaatacacataccaaaacaaagtggtgaaggttctcagaccagctgaaggagggaaaacagtcatggacatcatctcaagacaagaccagagagggagcggacaagttgttacttatgctctcaacacattcaccaacctggtggtgcagcttatccggaacatggaggctgaggaggtgctagagatgcatgatctatggctgttgaggaagccagagaaagtgaccagatggttgcagagcaatggatgggacagactcaaacgaatggcagtcagtggagatgactgcgttgtaaagccaattgatgataggtttgcacatgccctcaggttcttgaatgacatgggaaaagttaggaaagacacacaggaatggaaaccctcgactggatggagcaattgggaagaagtcccgttctgttcccaccacttcaacaagctgcacctcaaggatgggagatccattgtggtcccctgccgccaccaagatgaactgattggccgagcccgtgtctcaccaggggcaggatggagcatccgggagactgcctgtcttgcaaaatcatatgcccagatgtggcagcttctttatttccacagaagagacctccgactgatggccaatgccatctgttcggccgtgccagccgactgggtcccaactgggagaaccacctggtcaatccatggaaagggagaatggatgactaatgaggacatgctcatggtgtggaatagagtgtggattgaggagaacgaccacatgggggacaagacccctgtaacaaaatggacagacattccctatttgggaaaaagggaggacttatggtgtggatcccttatagggcacagacctcgcaccacttgggctgagaacatcaaagacacagtcaacatggtgcgtaggatcataggtgatgaagaaaggtacatggactacctatccacccaggtacgctacttgggtgaggaggggtccacacctggagtgctg

SEQ ID NO: 12 - DNA, 10269 bp, Zika virus
  misc_feature (1)...(10269): n = A,T,C or G
atgaaaaacccaaaaaagaaatccggaggattccggattgtcaatatgctaaaacgcggagtagcccgtgtgagcccctttgggggcttgaagaggctaccagctggacttctgctgggtcatggacccatcaggatggtcttggcgatactagccttcttgagattcacggcaatcaagccatcactgggtctcatcaatagatggggttccgtggggaaaaaagaggctatggaaataataaagaagttcaagaaagatctggctgccatgctgagaataatcaatgctaggaaggagaagaagagacgtggcgcagacaccagtgtcggaattgttggcctcctgctgaccacagccatggcagtggaggtcaccagacgtgggagtgcatactatatgtacttagacagaagcgatgctggggaggccatatcttttccaaccacactgggggtgaataagtgttacatacagatcatggatcttggacacatgtgtgatgccacaatgagctatgaatgccctatgttggatgagggggtagaaccagatgacgtcgattgctggtgcaacacgacatcgacttgggttgtgtacggaacctgccatcacaaaaaaggtgaggcacggagatctagaagagctgtgacgctcccctctcattccactaggaagctgcaaacgcggtcgcagacctggttggaatcaagagaatacacaaagcacttgatcagagtcgaaaattggatattcaggaaccctggctttgcgttggcagcagctgccattgcttggcttttgggaagctcaacgagccaaaaagtcatatacttggtcatgatactgttgattgccccggcatacagtatcaggtgcataggagtcagcaatagggattttgtggaaggtatgtcaggtgggacctgggttgatgttgtcttggaacatggaggttgtgttaccgtaatggcacaggacaagccaactgttgatatagagttggtcacaacaacggttagcaacatggcggaggtaagatcctactgctacgaggcatcaatatcggacatggcttcggacagccgctgcccaacacaaggtgaagcctaccttgacaagcagtcagacactcaatatgtttgcaaaagaacgttagtggacagaggttggggaaatggatgtggactctttggcaaagggagcctggtgacatgcgccaagtttgcatgctccaagaaaatgactgggaagagcatccagccagagaacctggagtaccggataatgctgtcagttcatggctcccagcacagtgggatgattgttaatgacanaggacatgaaactgatgagaatagagcgaaggttgagataacgcccaattcaccaagagccgaagccaccctgggaggttttggaagcctaggacttgattgtgaaccgaggacaggccttgacttttcagatttgtattacttgactatgaataacaagcattggttggtgcacaaggagtggttccatgacattccactaccttggcatgctggggcagacaccggaactccacattggaacaacaaagaagcattggtagagttcaaggacgcacatgccaaaaggcaaactgtcgtggttctagggagtcaagaaggagccgttcacacggctcttgctggagccctggaggctgagatggatggtgcaaagggaaggctgtcctctggccacttgaaatgtcgcttgaaaatggacaaacttagattgaagggcgtgtcatactccttatgtaccgcggcgttcacattcaccaagatcccggctgaaacgctgcatgggacagtcacagtggaggtacagtatgcagggacagatggaccctgcaaggttccagctcagatggcggtggatatgcaaactctgaccccagttgggaggttgataaccgctaaccctgtgatcactgaaagcactgagaattcaaagatgatgttggaacttgacccaccatttggggattcttacattgtcataggagttggggataagaagatcacccaccactggnacaggagtggcagcaccatcggaaaagcatttgaagccactgtgagaggcgccaagagaatggcagtcttgggagacacagcctgggactttggatcagtcggaggtgctctcaactcattgggcaagggcatccatcaaatttttggagcagctttcaaatcattgtttggaggaatgtcctggttctcacaaatcctcataggaacgttgctggtgtggttgggtctgaacacaaagaatggatctatttcccttacgtgcttggccttagggggagtgttgatcttcctatctacagccgtctctgctgatgtggggtgttcggtggacttctcaaagaaggaaacgagatgcggtacgggggtgttcgtctataacgacgttgaagcctggagggacaggtacaagtaccatcctgactcccctcgtagattggcagcagcagtcaagcaggcctgggaagatgggatctgtgggatctcctctgtttcaagaatggaaaacattatgtggagatcagtagaaggggagctcaacgcaattctggaagagaatggagttcaactgacggtcgttgtgggatctgtaaaaaaccccatgtggagaggtccgcagaggttgcctgtgcctgtgaatgagctgccccacggttggaaggcctgggggaaatcgtactttgtcagggcagcaaagaccaacaacagctttgttgtggatggtgacacactgaaggaatgcccgctcaaacacagagcatggaacagctttcttgtggaggatcacgggttcggggtatttcacactagtgtctggcttaaagtcagagaggattactcattagagtgtgatccagccgtcataggaacagctgctaagggaaaggaggccgtgcacagtgatctaggctactggattgagagtgaaaagaacgacacatggaggctgaagagggctcacctgatcgagatgaaaacatgtgaatggccaaagtcccacacactgtggacagatggaatagaagaaagtgatctgatcatacctaagtctttagctgggccactcagccaccacaacaccagagagggctacaggactcaagtgaaagggccgtggcatagtgaagagcttgaaatccggtttgaggaatgtccaggcaccaaggtccacgtggaggaaacatgtggaacgagaggaccgtccctgagatcaaccactgcaagcggaagggtgatcgaggaatggtgctgcagggaatgcacaatgcccccattgtcgttccgggcaaaagatggctgttggtatggaatggagataaggcccaggaaggaaccagagagtaacctagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgttggtagctatgatcctgggaggattttcaatgagtgacttggctaagcttgcaattctgatgggtgccaccttcgcggaaatgaacactggaggagatgtagctcatctggcgctgatagcggcattcaaagtcagacccgcgttgctggtctctttcatcttcagagccaattggacaccccgtgagagcatgctgctggccttggcctcgtgccttctgcaaactgngatctccgccctggaaggcgacctgatggttctcatcaatggttttgctttggcctggttggcaatacgagcgatggctgttccacgcactgacaacatcaccttggcaatcctggctgctctgacaccactggcccgaggcacactgcttgtagcgtggagagcaggccttgctacttgtggggggttcatgctcctctctctgaaggggaaaggtagtgtgaagaagaacctaccatttgtcatggccttgggactaaccgctgtgaggctggttgaccccatcaacgtggtgggactgctgttgctcacaaggagtgggaagcggagctggccccctagtgaagtactcacagctgttggcctgatatgtgcactggccggagggttcgccaaagcagatatagagatggctgggcccatggctgcagttggcctgctaattgttagttacgtggtctcaggaaagagtgtggacatgtacattgaaagagcaggtgacatcacatgggaaaaagatgcggaagttactggaaacagcccccggctcgatgtggcactagatgagagtggtgatttctccctggtggaggatgatggtccccccatgagagagatcatactcaaggtggtcctgatgaccatctgtggcatgaacccaatagccataccctttgcagctggagcgtggtatgtgtatgtgaagactggaaagaggagtggtgctctatgggatgtgcctgctcccaaggaagtaaaaaagggggagaccacagatggagtgtatagagtgatgactcgcagactgctaggttcaacacaagttggagtgggagtcatgcaagagggggtcttccacactatgtggcacgtcacaaaaggatccgcgctgaggagcggtgaagggagacttgatccatactggggagatgttaagcaggatctggtgtcatactgtggcccgtggaagctagatgccgcttgggacggacacagcgaggtgcagcttttggccgtgccccccggagagagagcgaggaacatccagactctgcccggaatattcaagacaaaggatggggacatcggagcagttgctctggactacccagcaggaacttcaggatctccgatcctagacaagtgtgggagagtgataggactctatggcaatggggtcgtgatcaaaaatggaagttatgttagtgccatcacccaagggaggagggaggaagagactcctgttgaatgcttcgaaccttcgatgctgaagaagaagcagctaactgtcttggatctgcatcctggagctgggaaaaccaggagagttcttcctgaaatagtccgtgaagccataaaaacaagactccgcacggtgatcctggctccaaccagggttgtcgctgctgaaatggaggaagcccttagagggcttccagtgcgttacatgacaacagcagttaatgtcacccactctgggacagaaatcgttgatttaatgtgccatgccaccttcacttcacgcctactacaacccattagagtccccaactacaatctttacattatggatgaggcccacttcacagatccctcaagtatagcagcaagaggatacatatcaacaagggttgagatgggcgaggcggctgccatcttcatgaccgccacaccaccaggaacccgcgacgcatttccggactctaactcaccaatcatggacacagaagtggaagtcccagagagagcctggagctcaggctttgattgggtgacggatcattctggaaaaacagtttggtttgttccaagcgtgaggaacggcaacgagatcgcggcttgtctgacaaaagctggaaaacgggtcatacagctcagcagaaagacttttgagacagagttccagaaaacaaaaaatcaagagtgggacttcgtcgtaacaactgacatctcagagatgggcgccaacttcaaagctgaccgggtcatagattccaggagatgcctgaagccggtcatacttgatggcgagagagtcattctggctggacccatgcctgtcacacatgccagcgctgcccagaggagggggcgcataggcaggaatcccaacaaacctggagatgagtatatgtatggaggtgggtgcgcagagactgatgaagaccatgcacactggcttgaagcaagaatgcttcttgataacatttacctccaagatggcctcatagcctcgctctatcgacctgaggccgataaggtagcagccattgagggagagttcaagcttaggacggagcaaaggaagacctttgtggaactcatgaaaagaggagatcttcctgtttggctggcctatcaggttgcatctgccggaataacctacacagatagaagatggtgttttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagaatttgccgctgggaaaagaggagcggcctttggagtgatggaagccctgggaacactgccaggacacatgacagagaggtttcaggaagccattgacaacctcgctgtgctcatgcgggcagagactggaagcaggccctacaaagccgcggcggcccaattaccggagaccttagagaccatcatgcttttgggtttgctgggaacagtctcgctgggaatcttctttgtcttgatgcggaacaagggcatagggaagatgggctttggaatggtgacccttggggccagtgcatggcttatgtggctctcggaaattgagccagccagaattgcatgtgtcctcattgtcgtgtttctattgctggtggtgctcatacctgagccagaaaagcagagatctccccaggacaaccaaatggcaattatcatcatggtagcagtgggtcttctgggcttgataaccgccaatgaactcggatggttggagagaacaaaaagtgacctaggccatctaatgggaaggagagaggagggggcaaccatgggattctcaatggacattgacttgcggccagcctcagcttgggctatctatgccgctctgacaactctcatcaccccagccgtccaacatgcggtaaccacttcatacaacaactactccttaatggcgatggccacgcaagccggagtgttgtttggcatgggcaaagggatgccattctatgcgtgggacttcggagtcccgctgctaatgatgggttgctactcacaattaacacccttgaccttaatagtggccatcattctgctcgtggcgcactacatgtacttgatcccaggtctacaggcagcagcggcgcgcgctgcccagaagagaacggcagctggcatcatgaagaaccctgttgtggatggaatagtggtgactgacattgacacaatgacaattgacccccaagtggagaaaaagatgggacaagtgctactcatagcagtagccatctccagtgccgttctgctgcgcaccgcctgggggtggggggaggctggggccctgatcacagccgcaacttccactttgtgggaaggctctccgaataaatactggaactcctccacagccacttcactgtgtaacatttttaggggaagttacttggctggagcttctcttatttacacagtaacaagaaacgctggcctggtcaagagacgtggaggtggaacgggagagaccctgggggagaaatggaaggcccgcctgaaccagatgtcggccctggagttttactcctacaaaaagtcaggcatcaccgaagtgtgcagagaagaagcccgccgcgccctcaaggacggagtggcaacaggaggccatgctgtgtcccgaggaagcgcaaagcttagatggttggtggagagaggatacctgcagccctatggaaaggtcattgatcttggatgtggcagagggggctggagttactacgccgccaccatccgcaaagttcaagaggtgaaaggatacacaaagggaggccctggtcatgaagaacccacgttggtgcaaagctatggatggaacatagtccgtcttaagagtggggtggacgtctttcacatggcggcggagtcgtgtgacactttgctgtgtgacataggtgagtcatcatctagtcctgaagtggaagaagcacggacgctcagagtactctccatggtgggggattggcttgaaaaaagaccaggggccttttgtataaaggtgttgtgcccatacaccagcaccatgatggaaaccctagagcgactgcagcgtaggtatgggggaggactggtcagagtgccactctcccgcaactctacacatgagatgtactgggtctctggagcgaaaagcaacatcataaaaagtgtgtccaccacgagccagctcctcttgggacgcatggacgggcccaggaggccagtgaaatatgaggaggatgtgaatctcggctccggcacgcgagctgtggcaagctgcgccgaagctcccaacctgaagatcattggtaaccgcgttgagaggatccgcagtgagcatgcggaaacgtggttctttgatgagaaccacccatacaggacatgggcttaccatgggagctacgaggcccctacacaagggtcagcgtcttctctcataaacggggttgtcaggctcctgtcaaagccctgggatgtggtgactggagtcacaggaatagccatgaccgacaccacaccgtatggccagcaaagagttttcaaggaaaaagtggacactagggtgccagacccccaggaaggcactcgtcaggtgatgaacatggtctcttcctggctatggaaggagctaggtaaacacaaacggccacgagtttgcaccaaagaagagttcatcaataaggttcgcagcaatgcagcactgggggcaatatttgaagaggagaaagaatggaagactgcagtggaagctgtgaacgatccaaggttctgggccctagtggacaaggaaagagagcaccacttgagaggagagtgtcagagctgtgtgtacaacatgatgggaaaaagagaaaagaagcaaggggaatttggaaaggccaagggcagccgcgccatttggtacatgtggctaggggctagatttctagagtttgaagcccttggattcttgaacgaggatcactggatggggagagagaattcaggaggtggtgttgaagggctgggattacaaagacttggatatgttctagaagaaatgagccgcacaccaggaggaaagatgtatgcagatgataccgctggctgggacacccgcatcagtaggtttgatctggagaatgaagctctgatcaccaaccaaatggagaaagggcacagggccttggcgttggccataatcaagtacacataccaaaacaaagtggtaaaggtccttagaccagctgaaagagggaagacagttatggacatcatctcaagacaagaccaaagagggagcggacaagttgttacttacgctcttaatacattcaccaacctggtggtgcagctcattcggaacatggaggctgaggaagttctagagatgcaagacttgtggctgttgaggaggccagagaaggtgaccagctggttgcagagcaacggatgggataggctcaaacgaatggcagtcagtggagatgattgtgttgtgaaaccaattgatgataggtttgcacatgccctcaggtttttgaatgacatggggaaagttaggaaggacacacaggagtggaaaccctcaactggatggagcaactgggaagaagttccgttttgctcccatcacttcaacaagctttacctcaaggacgggaggtccattgtggtcccctgtcgccaccaagatgaactgattggccgagcccgcgtctcaccaggggcgggatggagcatccgggagactgcttgcctagcaaaatcatatgcacaaatgtggcagcttctttatttccacagaagggacctccgactgatggccaacgccatttgttcatctgtgccagttgactgggttccaactgggagaaccacctggtcaatccatggaaagggagaatggatgaccactgaggacatgcttgtggtgtggaacagagtgtggattgaggagaacgaccacatggaggacaagaccccagtcacgaaatggacagacattccctatttgggaaaaagggaagacttatggtgtggatctcttatagggcacagaccacgcactacttgggctgagaacattaaagacacagtcaacatggtgcgcaggatcataggtgatgaagaaaagtacatggactacctatccactcaagttcgctacttgggtgaagaagggtccacacctggagtgtta

SEQ ID NO: 13 - DNA, 10290 bp, Zika virus
atgaaaaacccaaaaagagccggtagcagccggcttgtcaatatgctaagacgcggtgcagcccgtgtcatccctccaggaggagggctcaagaggctgcctgtaggattgctgttgggtcggggtccgatcaaaatgatcctggccatactggcattcctacgatttacagcaataaaaccgtccactggcctcatcaacagatggggaaaagtgggcaaaaaagaggccatcaaaatcctcacaaaattcaaggctgacgtgggcaccatgctgcgtatcatcaacaatcggaagacaaaaaagagaggagtcgaaactggaattgtgttcctggcattgctggtgtctattgttgctgtggaagtcacaaaaaagggggacacctattacatgtttgcggacaagaaggacgccggaaaggtggtgacctttgagactgaatctggacccaaccgttgctccatccaagcaatggacattggacatatgtgtccagctacaatgagctatgaatgtcccgtgctggaaccacagtatgagccagaggatgtcgactgttggtgcaactcgacagcagcatggattgtgtatggcacatgcacccacaagacaacgggagagacaagacgttccagacgttcaatcaccctgccatctcatgcctcacaaaagttggagaccagatcatcgacgtggcttgaatcccgcgaatactccaaatatctaataaaggtggaaaactggatcctccgcaatccaggatatgcgttggtggctgcagtgattggatggactctgggcagcagtcgcagccagaagatcatctttgtcactctgctcatgttggtagcccccgcatacagcatcagatgcattggaattggaaacagagacttcattgagggaatgtccggtggcacctgggtggacattgtcctggaacatggtggttgtgtgacagtaatgtcaaacgacaaacccacattggactttgaactggtgacaacgaccgcaagtaacatggctgaggtcaggtcctactgctatgaagctaacatatccgagatggcatcggacagcaggtgccccacacagggggaagcttatcttgacaaaatggccgactcccagtttgtgtgcaagcgtgggtacgttgacaggggctggggaaacggatgtggactctttggaaaaggaagcattgtcacttgcgctaagttcacgtgtgtgaaaaagctcacagggaaaagcattcaaccggagaatctcgagtaccgggtccttgtttcggtgcacgcttcccaacatggaggaatgattaacaatgacaccaatcaccaacacgacaaggagaacagagcgcgcattgatatcacagctagcgctccccgtgttgaggtggaacttggctcctttggatccttctcgatggagtgtgaaccccggtcaggattgaactttggtgacctgtattacctcaccatgaacaacaagcattggctggttaatagagattggtttcacgatctttccttgccatggcatacaggagccacatcaaacaatcatcactggaacaacaaggaggcgctggtagaattcagagaagcccacgcaaagaagcagacggctgtggtcctgggaagtcaggaaggagctgttcacgcagcactggccggcgcactggaggctgagtctgatggacacaaagcgactatctactctggacacttgaagtgtcgcttgaagctagacaaactgcgcctgaagggaatgtcatatgcactctgcacaggagcattcaccttcgctcgcaccccctctgaaacaattcacggcaccgccacagtggagctgcaatatgcaggtgaagatgggccgtgcaaagttcccatagtaattaccagtgacaccaatagcatggcctcgacaggcaggctgatcacagcgaatccggtggtcacggaaagtggagcaaactcaaagatgatggtcgagattgaccctccgtttggtgattcttacattattgtgggcactggcacaacaaaaattacccaccattggcacagagccggtagttcaattggacgtgcatttgaggctaccatgagaggagcaaaacggatggcggtcctcggcgacaccgcttgggactttggctctgttgggggcatgttcaactccgttggaaagtttgtccaccaggtgtttggatcagcatttaaggcattgtttggaggcatgtcctggttcacacagctcctgataggatttctgctcatatggatgggtttgaacgcacgcggtggaaccgtggccatgagcttcatgggcattggggctatgctgattttcctagccacctcggtgtcaggagacacaggatgctcggttgacatatccagaagggaaatgcggtgcgggagcggcatattcgtgtacaatgacgttgacgcatggcgaagccgctacaaataccatcctgaaacccccagagctttggccgctgccgtgaaaacggcttgggaagaagggacctgtggcattacctcagtgagcagaatggaaaacctgatgtggagctctgtggctggagagttgaatgcaatccttgaggacaattcagtgccattgacagtcgtcgttggcgagccaaaatatccactgtacaatgctccaaagaggctgaaaccaccagcatcagagttaccgcaggggtggaagtcctggggaaagtcatactttgtctcagccgcaaaaaacaacaactcctttgtggtagatggtgacaccatgaaggaatgcccaagacagaagcgagcatggaacagcttgagaatagaggatcatgggttcggagtcttccacactagcatctggctgaaattccatgaggacaactccaccgaatgtgacacagctatcataggaacggcggttcgcgggaaggaagccgttcatagtgacttgggctactggatagagagtgagcgcaatgacacatggaggctctctcgagcgcacctgatcgaagcaaagacatgtgaatggccacggtcgcacacactgtggacggacggagtggaagagagcgagctgatcattccacgtggcttagccggtcctttcagccatcataacacgcgtgctggctacaagactcagaataaaggtccctggcatttaggtgatgttgaaattcagttcgccacgtgccccggaacaaccgtggtccaggaccaagagtgcagggacaggggcgcttctctacgcacgaccacagctagtggaagggtaatcaatgaatggtgctgcaggtcgtgcaccatgcctccactcagtttcaagacaaaagatggatgttggtatgcaatggagatacgtcctgtgaaagaacaagagtcaaacctcgtgcgatcgcacgtcactgccggaagcacagaccacatggaccatttctctctcggattagtagtggtcatgttgatggtgcaagaaggtatgaagaagagaatgacatcaaaagcaataatcacctcagcggcctttctcctggcggttatgatagtgggaggtttcacgtaccaggattttgggaggctggtggtattggtgggtgctgcatttgctgagatgaacactggaggtgacgttgcgcacctggcgctggtggcagcgtttaaagtgaggccagcgatgctggtctcattcatgttcagagccttgtggacccccagggagtcactgcttttagctctggctgcctgcctcctgcaggtgtcagtgacaccactggatcattccatcatgatcgtggttgatgggattgcgctgtcctggttgtgtctgaaagccatcttggtgccgcgtaccccaaacatagcccttcctcttctcgctatgctgtcacccatgctccaaggtaccaccattgtggcatggcgagctatgatggcggccctggctgtcataaccttggcttccatgaagcatggaaggggtgtaaaaaagacgtttccctacaccatcggatgcatccttggcagcatgggcttagttgaaaacttggggttggttggcctcctcttgttgacagcctcaaaaaagaggagttggcctccgagtgaggtgatgacggctgtcggactgatctgtgcaattgtgggcggactaaccaagaccgacattgacatggcgggacccatggcagccataggactgctggtggtgagctatgtggtttctggcaagagtgtggacatgtacattgaaaaggtgtgtgacatatcatgggacaaggacgctgaaataacaggcacaagtccgcggctggatgtggctctcgacgacagtggagatttctcacttatccaggatgacgggccccccactcgagagattgtgttgaaggtgtttctgatgtgtgtttgcggtgtcagccccatagccatcccctttgcagccgctgcttggttcgtgtacattaaatcagggaaaagaagcggcgccatgtgggacattccatccccaagagaagtgaaaaaaggggaaacaacggctggagtgtacagaatcatgacgcgtaaattgctgggcagcacacaggtgggagccggagtaatgcatgaaggtgtttttcacacaatgtggcacgtcacaaaaggttcggcccttcggagtggtgagggacgcctagatccatactggggaaacgtgaagcaggatttgatctcttactgcggaccatggaaactggatgggaaatgggacggcgtgtcggaagtccaactgatagcggtcgccccaggtgagcgcgccagaaatgtgcagacaaaaccaggagtgttcaagaccactgatggggaaatcggggccttggcccttgacttcccaggcggaagttcaggctccccgataattgacaaaaatggacatgtaattggcctgtatggaaatggtgtcgtggtcaagagtggaagctacgtgagtgccatcatgcagacagagaagatggaggaacccgcagttgactgctttgaggaggacatgctgagaaaaaagaagctgacggtgctcgacctccatccaggagctggaaaaactcgaagagtgctccctcagatcgtcaaggctgcaattaagaaacgcctacgcacggtaatcctggcacccacccgagtggtggcagctgagatggctgaggcactaaaagaccttccaataaggtacatgactccggcagtttcagccacccatgatggcaatgagattgttgaccttatgtgccacgccacttttacatcaaggctaatgcaaccaattagggtgcctaattacaatctatatataatggatgaggcccacttcacagatcctgcaagcatcgctgcaagagggtacatagcaacaagagtggacatgggagacgccgcggccatcttcatgacggccacccctcctggcagcactgaagctttcccggattcaaacgcccccatcacagatgttgaaacagaggttcctgacaaggcgtggaattctggatttgaatggatcactgattacccagggaaaaccgtttggtttgtccctagtgtcagaatgggcaatgagatctcggcctgcctcacaaaagccggcaaatcggttatccaactcagccggaaaacctttgaaacagagtaccagaagacaaagaatggtgagtgggactttgtcgtgaccactgacatctcagaaatgggagccaacttcaaggccgacagagtcatagactcacggaaatgcttgaagccagtgattctggatgacatggaagagagagttgttcttgccgggccgatggcagtaacaccatccagcgcagctcaacgcagaggaagaattggaagaaaccccaacaaaactggagatgagttctattacggggggggctgtgccgcaacggatgatgaccatgctcattgggtagaggctaggatgctgcttgacaacatctacctccaggacaacctcgttgcatctctgtacaagccagaacaaggaaaggtctcggcaatagaaggggagttcaaactgagaggagaacagaggaaaaccttcgtggagctgatgaagagaggggacttgccagtgtggttgtcatatcaagtggcggcctccggactcagctatactgaccggcgctggtgctttgatggaaaaaacaacaacaccatcctggaggactgcgtccccgtcgaggtgtggacaaaatttggagagaaaaagattctgaagcccagatggatggacgctcggatctgctctgatcatgcctctttgaagtctttcaaggagtttgctgcaggaaagagaacaatagccactggcttaattgaggcttttgggatgcttcccgggcacatgactgagagattccaggaggccgtcgacaatttggccgtgttgatgagggccgaggcaggctctagggcacacagaatggctgcagcacagctccctgagacaatggaaaccatcctgctcctcagcctgctggcattcgtgtcacttggtgtattttttgtactgatgagggcaaaagggttaggaaaaatggggtccggcatgatcgtgctggcaggaagtggctggctcatgtggatgtctgaggtggaaccagcccgcatagcttgtgtggtgatcatagtgtttctgctaatggtcgttctgattccggaaccggagaagcagcgctctccccaggacaatcagctggctctaattatcttgatcgcgacgggcctcatcacgctcatcgcggccaatgagctgggttggttagaaagaacaaagagtgacctcaccaggctgttttggagagaacacgctgagccaacaggagggagagggttttccttctcgctggacattgacctgcggccggcatcggcctgggcaatatatgccgctatgacaaccctgatcacaccgacagtccaacacgctgtgaccacatcgtacaacaactactctctcatggctatggccactcaggccggagttctttttggcatgggacggggggtgcctttttacaaatgggactttggcgtgccactccttatgctgggctgctactcacaacttaccccactcaccctgatcgtggctctcgtgatgctagccgctcactatctctatctcatccccgggctccaggcaacggccgccagggccgcccaacgaaggacggctgctggaataatgaaaaacccagtggtggatggaattgtggtaactgacatagacccaatccaaatcgatccaaatgtcgaaaagaagatgggccaggtcatgctcatctttgtggctttggcgagcgcggttctcatgagaacggcatggggttggggagaggctggtgcccttgcatcggcagcagctgccaccctatgggaaggggctcccaacaagtactggaattcatcaacggctacatccttgtgcaacatatttcggggaagttatctggcaggtccctccctcatctacaccgtcacacgcaatgcaggtatcatgaagaaaaggggcggtggaaatggagaaacggtgggcgagaaatggaaggagcgcttgaatcggatgaccgcgcttgaattctacgcctacaagcggtcaggaataactgaagtgtgcagagaacccgccagaagagccttgaaggatggagtcgtcacaggaggacacgctgtctcccgcggaagcgcaaagctgcgatggatggtggaacgtggccacgtcaatctagtgggacgcgttgtcgacctcggatgtggaaggggtggctggagttactacgccgcatctcaaaagcaagtcctcgaggtgagaggctacacaaaagggggagcgggccacgaggagcccatgaatgtccaaagttatggttggaacatagtgcgactcaagagtggagtggacgttttttatctaccatcagaaccatgtgacacgctgctctgtgacattggagagtcatcctcgagcccagcagtggaagaagcccggactctgagagtgctcgggatggttgaaacctggctggaacggggcgtaaagaacttctgcatcaaagtgctctgcccgtacaccagtgccatgattgagcggctggaagccctccagcgtcgctacggaggaggcctggtgagggttccactctccagaaattccacccacgaaatgtactgggtctctggagcaaaatcaaacatcatcaggagtgtgaatgccaccagccagctgctcatgcacagaatggacatccccacgcggaaaacaaagtttgaagaagacgtcaatctggggaccggaaccagggcagttgaaagcagagctgaccctcccgacatgaaaaaactaggcagccggattgagcggttgagaaaggaatatggatccacttggcactacgatgaaaaccacccctacaggacatggcattaccacggcagttatgaggctgacacgcaaggctccgcctcctcaatggtcaacggcgtggtgcgtctcctctcaaaaccatgggatgcattgagctcggtcaccaacattgctatgacggacacaactccgtttggacagcagcgggtgttcaaggagaaagtggacacccggactccagaccccaagcagggcacgcaaagagtcatggccataacatcacaatggctgtgggaccgcctagcaagaaacaagacccctcggatgtgcacgcgacaggaattcataaacaaggtcaacagtcacgcggcgttgggacccgtttttagagaacagcagggatggggttcagcggccgaagcggtggtagatcctaggttttgggagctcgttgacaatgaaagagaagcccatttgagaggggagtgcttgacctgtgtctacaacatgatggggaaaagagaaaagaagctcggtgaattcgggaaggcaaaaggcagcagagccatttggtacatgtggctgggagcccgcttcctcgagttcgaggccctgggcttcctcaatgaagaccactggttaagcagagagaactctggagggggagttgagggcttgggcctccaaaaacttggatacatccttgaagagatcagcaggaggccaggaggcaaaatgtatgccgatgacacggctggctgggacacccgcatcacgaaatgcgacctagaaaatgaggcgcgcattttggaaaaaatggacgggatccacaaaaaactcgcacgggccgtcatcgagttgacatacaagcataaggttgtgagagtcttgagaccagcaccacaagggaaggtcgttatggacatcatctccaggccagaccaaagggggagtgggcaggtggttacttatgccctcaacacctatacaaacttggtggtgcagctgatccgtaacatggaagcagaggctgtcatcaatgaaagagacatggaggagctccaaaacccatggaaagtcatcaattggctagaaggaaatggatgggacagactccgctcgatggcagtgagtggagatgactgtgtcgtgaaaccaatggatgataggttcgcctatgcactgaatttcctcaatgacatgggcaaggtcagaaaagatgtccaggaatggaagccctcgccggggtggacaaactgggaagaagtgcccttttgctcccaccacttcaacaagctcccgatgaaggatggaagaacaataatagttccctgccggcaccaagatgagttgataggcagggctagagtttctccaggaaaaggctggtcactcagtgaaacagcatgcttgggcaagtcttatgcccagatgtggctactgttgtactttcacaggagagatctccgactcatggcaaacgcaatctgctctgctgtaccggtgagttgggtgcccacggggagaacaacctggtccatccatgggcgtggagagtggatgacaacagaggacatgctagaggtatggaacagagtgtggatcatagagaatgagtacatggaggacaagacccctgtcacagagtggaccgatgttccatacttgggaaagagagaagacttgtggtgcggctcccttattggacacaggccaagaagcacatgggcagagaacatctgggctgccatttatcaagtgcgccgagcaatcggcgaaactgaagaatatagagactacatgagcacacaggtccgctatggctcggaggaagggccaagcgctggtgtgttgtaa

SEQ ID NO: 14 - DNA, 4251 bp, Artificial Sequence
  A synthetic oligonucleotide
  misc_feature (1)...(4251): n = A,T,C or G
acgaccgaagcgcancgagntcagtgagcgaggaagcggaagagcgcccaannnnnnnnnnnnnnnnnnnnnnnnnnnnnnnnattcattaatgcagaattccttccgagtgagagacacaaaaaattccaacacactattgcaatgaaaannnnnnnnnnnnnnnnnnnnnnnnnnnnnnnntcaaggggcttcatgatgtccccataatttttggcagagggaaaaagatcatacgtagatcgaaacgannnnnnnnnnnnnnnnnnnnnnnnnnnnnnnncagttcctatgagctttgctccagaccggctgcgagcaaaacaagcggctaggagttccgcagtatggnnnnnnnnnnnnnnnnnnnnnnnnnnnnnnnnccgctgatggcctatggccaagccccagccagtgggggttgcgtcagcaaacacttggcacagaccagnnnnnnnnnnnnnnnnnnnnnnnnnnnnnnnntactgtttacttagaaaggccttgtaagttggcgagaaagtgaaagcctgtttagcttgtatacatgcnnnnnnnnnnnnnnnnnnnnnnnnnnnnnnnnctgtaaatggagcagcaaagcccaaaagacccacaatcctttgacatactttccaatcaataggcctgnnnnnnnnnnnnnnnnnnnnnnnnnnnnnnnncttttgtaacctatgatcttgtggcaatgtcccccaacttccaattacgtaacccatgaagtttagggnnnnnnnnnnnnnnnnnnnnnnnnnnnnnnnnagatcctcacaccgagcaccccacatcagcagagacagctgtggataagaagatcaacactccccctannnnnnnnnnnnnnnnnnnnnnnnnnnnnnnntttgtgttcagacccaaccacatcagcaacgttccaatgagaatttgtgagaaccaggacattcctccnnnnnnnnnnnnnnnnnnnnnnnnnnnnnnnncatggatgcccttgcccaatgagttgagagcgcctccaacttagccaaagtcccaggctgtgtctcccnnnnnnnnnnnnnnnnnnnnnnnnnnnnnnnncgcttcaaatgcttttccaatggtgcgtccactcctgtgccagtggtgggtgatcttcttctccccgannnnnnnnnnnnnnnnnnnnnnnnnnnnnnnncgatcaagttccagcatcatcttagagttctcagtgctttcagtgattacggggttagcggttatcaannnnnnnnnnnnnnnnnnnnnnnnnnnnnnnnccgccatctgacgtggaaccttgcaaggtccatctgtccctgcgtactgtacctccactgtcactgtcnnnnnnnnnnnnnnnnnnnnnnnnnnnnnnnntgtgaacgctgcggtacacaaggagtatgacacgcccttcaatctaagtttatccattttcaggcgacnnnnnnnnnnnnnnnnnnnnnnnnnnnnnnnnccaccatccatctcagcctccagagctccagcaagggccgtgtgaactgctccttcttgactccctagnnnnnnnnnnnnnnnnnnnnnnnnnnnnnnnnccttgaactctaccagtgcttctttgttgttccagtgtggagttccggtgtctgccccagcgtgccaannnnnnnnnnnnnnnnnnnnnnnnnnnnnnnnaaccaaccagtgcttgttattcatagtcaagtaatacaaattcgaaaagtcaaggcctgtcctcggttnnnnnnnnnnnnnnnnnnnnnnnnnnnnnnnnagggtggcttcggctcttggtgaattgggcgttatctcaaccttcgctctattctcatcagtttcatgnnnnnnnnnnnnnnnnnnnnnnnnnnnnnnnncggagccatgaactgacagcattatccggtactccagattctctggctggatgctcttcccggtcattnnnnnnnnnnnnnnnnnnnnnnnnnnnnnnnncaggctccctttgccaaaaagtccacatccatttccccagcctctgtccactaagcttcttttgcagannnnnnnnnnnnnnnnnnnnnnnnnnnnnnnnccttcaccttgtgttgggcagcggctgtccgaagccatgtcctagatttaggcctcatagcagtaggannnnnnnnnnnnnnnnnnnnnnnnnnnnnnnntaaccagctctatgtcgacagtcggtttgtccgtggccattacggtgacacaacctccatgttccaagnnnnnnnnnnnnnnnnnnnnnnnnnnnnnnnnttccacaaagtccctattgctgactcctatgcacctgatgctgtagtccggggcaatcagcagtatcannnnnnnnnnnnnnnnnnnnnnnnnnnnnnnncttcccaaaagccaagcgatggcagctgctgctaacgcgaagccagggttcctgaatatccaattttcnnnnnnnnnnnnnnnnnnnnnnnnnnnnnnnnattccaaccaggtttgcgaccgcgtttgcagcttcctagtggaatgggaggggagcgtcacagctcttnnnnnnnnnnnnnnnnnnnnnnnnnnnnnnnnccaggttccgtacacaacccaagttgacgtcgtgttgcaccaacaatcgacgtcatctggttccacccnnnnnnnnnnnnnnnnnnnnnnnnnnnnnnnnctggcatcacacatgtgtccaagatccatgatctgtatataacacttattcatccccaatgtggttggnnnnnnnnnnnnnnnnnnnnnnnnnnnnnnnnccaagtacatatagtatgcactcccacgtctagtgacctccgctgccatagctgtggtcagcaggaggnnnnnnnnnnnnnnnnnnnnnnnnnnnnnnnnagggtccccagtcctcgcggagattgacgagatgtgagaggcaatattcggagcagggtttactgttcnnnnnnnnnnnnnnnnnnnnnnnnnnnnnnnncccctgactctgggatcctgacctggaagatagtcagggttgaggcaagcaaaaggtacatgtaagagnnnnnnnnnnnnnnnnnnnnnnnnnnnnnnnntcttgactggggaagccaggcccaccctggagagtacatacctgcttgctgagatccggacggtgagtnnnnnnnnnnnnnnnnnnnnnnnnnnnnnnnncaccgttcccggccgcggaggctggatcggtcccggtgtcttctatggaggtcaaaacagcgtggatgnnnnnnnnnnnnnnnnnnnnnnnnnnnnnnnncagctctgcttatatagacttcccaccgtacacgcctaccgcccatttgcgtcaacggggcggggttannnnnnnnnnnnnnnnnnnnnnnnnnnnnnnnctgccaaaacaaactcccattgacgtcaatggggtggagacttggaaatccccgtgagtcaaaccgctnnnnnnnnnnnnnnnnnnnnnnnnnnnnnnnnatcaccatggtaatagcgatgactaatacgtagatgtactgccaagtaggaaagtcccgtaaggtcatnnnnnnnnnnnnnnnnnnnnnnnnnnnnnnnncgtcattgacgtcaatagggggcggacttggcatatgatacacttgatgtactgccaagtgggcagttnnnnnnnnnnnnnnnnnnnnnnnnnnnnnnnncaaagtccctattggcgttactatgggaacatacgtcattattgacgtcaatgggcgggggtcgttggnnnnnnnnnnnnnnnnnnnnnnnnnnnnnnnntatgtaacgcggaactccatatatgggctatgaactaatgacccctgaattgattactattaataactnnnnnnnnnnnnnnnnnnnnnnnnnnnnnnnnccctgacatcgcgaagcagcgcaaaacgcctaaccctaagcagattcttcatgcaattgtcggtcaagnnnnnnnnnnnnnnnnnnnnnnnnnnnnnnnnccactactcagcgacctccaacacacaagcagggagcagatactggcttaactatgcggcatcagagcnnnnnnnnnnnnnnnnnnnnnnnnnnnnnnnncgagatctcccgatccgncgacgtcaggtggcacttttcggggnaatgngcggggaacccnnanttntnnnnnnnnnnnnnnnnnnnnnnnnnnnnnnnnngcgtcatgagacaataaccctgataaatgcttcaataatatgaaaaaggaagagtatgagtatcaaa